(19) United States
(12) Patent Application Publication
(10) Pub. No.: US 2025/0255951 A1
(43) Pub. Date: Aug. 14, 2025

(54) Vaccines against Chlamydia sp.

(71) Applicant: STATENS SERUM INSTITUT, Copenhagen S (DK)

(72) Inventors: Frank Follmann, Soborg (DK); Ida Rosenkrands, Vaerlose (DK); Anja Olsen, Soborg (DK); Peter Andersen, Bronshoj (DK)

(73) Assignee: STATENS SERUM INSTITUT, Copenhagen S (DK)

(21) Appl. No.: 19/197,254

(22) Filed: May 2, 2025

Related U.S. Application Data
(62) Division of application No. 17/155,264, filed on Jan. 22, 2021.
Application No. 17/155,264 is a continuation of application No. 15/956,731, filed on Apr. 18, 2018, now Pat. No. 10,925,954.
Application No. 15/956,731 is a continuation of application No. 14/216,403, filed on Mar. 17, 2014, now abandoned.
(60) Provisional application No. 61/802,907, filed on Mar. 18, 2013.

(30) Foreign Application Priority Data
Mar. 18, 2013 (DK) PA 2013 00155
Dec. 11, 2013 (DK) PA 2013 00684

(51) Int. Cl. A61K 39/118; A61K 39/00; C07K 14/295

(52) U.S. Cl. CPC A61K 39/118; C07K 14/295; A61K 2039/6031; C07K 2319/40

(57) ABSTRACT

The present invention describes an efficient vaccine against a Chlamydia trachomatis (Ct). The vaccine is based on recombinant fusion molecules that are capable of generating a high titered neutralizing antibody response that is protective against various Ct serovars. Our invention furthermore describe the combination of these antibody promoting fragments with Ct antigens that are targets for T cells with the aim to provide a vaccine that activate both arms of the immune system.

### FIELD OF INVENTION

The present invention relates to polypeptides of repetitive units of immunogenic fragments of surface exposed regions of outer membrane proteins of Chlamydia sp. and pharmaceutical compositions and vaccines comprising these fusion proteins.

### BACKGROUND OF THE INVENTION

Chlamydiae are intracellular bacterial pathogens responsible for a variety of infections. Chlamydia pneumoniae is responsible for human acute respiratory infection and believed to play a role in coronary heart disease. Chlamydia trachomatis is the causative agent of human sexually transmitted disease and eye infections (Trachoma). Also in animals, several infections with Chlamydia sp. are known, e.g. Chlamydia suis infecting pigs, and Chlamydiaphila abortus which causes abortion in small ruminants (sheep and goats).

Worldwide, it is estimated that 92 million individuals become sexually infected with Chlamydia trachomatis (Ct) ¹. Urogenital infections with Ct are of public health concern because of its high prevalence and the fact that it's a risk factor for ectopic pregnancy and infertility ². In addition to this Ct infections have been shown to facilitate the transmission of HIV ³ and act as a co-factor in HPV-induced cervical carcinoma ⁴. The duration of untreated genital Ct infection can be prolonged, and complete clearance is often not reached within the first 12 months ⁵. From human studies it is known that some degree of protective immunity against genital re-infection develops, although it appears at best to be partial ⁶. The infection is effectively controlled by antibiotic therapy; however the high prevalence of asymptomatic cases suggests that sustainable disease control can only be envisaged if an effective Chlamydia vaccine is developed.

A vaccine against Ct needs to elicit protective T-cell and B-cell immunity in the genital tract mucosa 7. Immune mechanisms of clearance of infection and resistance to re-infection have been described in numerous studies. A variety of animal models and chlamydial species have been used in attempts to identify protective and damaging immune responses. A general consensus has emerged that, in mice, CD4+Th1 cell mediated immune responses plays a major role in the resolution of Ct infection^{8, 9, 10}, whereas the role of humoral immunity in protection has remained less well defined. In guinea pigs immunity to chlamydial infection is mediated at least partly by secretory IgA at the mucosal surface ^{11, 12} and also in the mouse model there is increasing evidence to support a role for antibodies in protective immunity ⁹. Data from animal models that has emerged over the last years clearly demonstrate that if antibodies are formed after the infection is established they play a minimal role, whereas their presence at the time of infection (e.g. in a secondary response) promotes significant levels of protection, an effect that is however clearly amplified in the presence of Chlamydia specific CD4⁺ cells^{9, 13, 14}. A strong cell mediated immune (CMI) response without antibodies may on the other hand control bacterial replication but can in the worst case exacerbate the pathology associated with Chlamydia infection ^{15 16}. The importance of this interplay between cell mediated immunity and antibodies is also becoming increasingly clear to support a preferential role of neutralizing antibodies in the initial phase of infection, whereas CD4⁺ cells are the main effectors throughout the rest of the infection ^{17 18 19}. In summary balancing the immune effector mechanisms between antibodies and T cells seems to be crucial for disease outcome.

We and others have identified a range of chlamydial antigens recognized during a natural infection in either humans or animal models ^{20, 21 22, 23 24 25, 26 27}. Especially the publishing of the genome sequence in 1998 and modern high throughput techniques have led to the testing of almost the entire genome of 875 open reading frames ²⁸. Importantly, identifying proteins as antigenic during an infection do not necessarily mean they are protective as vaccines ²⁹ and despite the characterization of such a large number of antigens only very few of these have been demonstrated to mediate protection as vaccines in animal models ^{30 31, 32} Furthermore for the majority of the vaccines recently reported the partial protection observed is mediated by T cells with no neutralizing antibodies. Therefore there is a lack of vaccine candidates that generate neutralizing antibodies that can cope with the infection in the initial phase and creating a balanced immune response.

Until now there has only been convincing data on neutralizing antibodies with three surface exposed antigens; PorB, which localized in the chlamydial outer membrane and functions as a porin ³³. Antibodies against this has been shown to neutralize chlamydial infectivity ³⁴, patent ref: U.S. Pat. No. 7,105,171. Another more recent antigen is PmpD. This protein has been shown to generate neutralizing antibodies in vitro, however the in vivo relevance of these antibodies have not yet been demonstrated ³⁵.

MOMP is the classical target antigen for neutralizing antibodies and one of the first antigenic molecules described. It is a surface-exposed trans membrane protein which has structural (porin) properties ^{36, 37, 38}. MOMP is a 40 kDa protein making up roughly 60% of the protein in the Ct membrane and is a target for neutralizing antibodies with proven efficacy both in vitro and in vivo. MOMP consists of four variable surface exposed domains (VD-1 to VD-4) separated by five constant segments ^{36 39} and it is the molecular basis of the serovar (˜15) grouping of Chlamydia (FIG. 1). The in vitro and in vivo neutralizing antibody epitopes have been mapped to these VDs ^{40 41 42 43 44}. The distribution profile of Ct urogenital serovars has been described for regions worldwide, providing epidemiological data for the serovar coverage needed of a MOMP based vaccine. The most common serovar detected worldwide is E (22-49% of cases) followed by serovars F and D (17-22% and 9-19%, respectively) ^{45 46 47 48 49 50}, meaning that a vaccine targeting serovars E, D and F would have a significant impact and cover more than 70% of the human population.

MOMP is highly immunogenic in humans and animals and has therefore been studied in great detail as a vaccine candidate, both as a natively purified protein, recombinantly and as DNA-vaccine. These vaccination attempts gave variable results ^{17, 51, 52, 53, 54, 55, 56, 57} The reason for the relative inconsistency of MOMP as a vaccine is not fully understood, but the fact that the synthetic MOMP immunogens do not mimic the native structure of the protein has been the major concern 54. In this regard, the structure of this membrane bound cysteine rich molecule and refolding various products to achieve native protein structure has been extremely challenging and is not suitable for large scale vaccine production ⁵⁸. Therefore, although clearly with vaccine potential, full size MOMP has so far not been a feasible vaccine candidate and several attempts have therefore been made to construct a vaccine based on selected epitopes (such as the highly conserved TTLNPTIAG (SEQ ID NO: 76) in VD4 ^{36, 59}) or based on selected regions rich in neutralizing target epitopes (such as the VD's) from MOMP (WO9406827, U.S. Pat. No. 6,384,206) ^{60, 61 62, 63 64 51, 65 66.}

There has been special focus on VD1, VD2 and VD4 because neutralizing monoclonal antibodies used for serotyping has been shown to map to these regions. These VD regions are targeted by antibodies during natural infection and in line with this, these regions have naturally been the focus of attempts to develop immuno-diagnostics. For example Mygind et al. constructed different polyantigens containing VD regions from different serovariants in the search for a diagnostic tool based on ELISA ⁶⁷. This analysis revealed that by increasing the number of serovariants and include the species specific TTLNPTIAG (SEQ ID NO: 76) into one recombinant polyantigen, it was possible to increase the specificity and sensitivity of the assay compared to an assay based on a single serovariant antigen.

Mainly VD4 has attracted interest as an immunogen because this region was shown to contain the highly conserved species-specific epitope TTLNPTIAG (SEQ ID NO: 76) embedded in the variable region. Importantly, this conserved epitope in the VD4 region can elicit a broadly cross-reactive immune response, which is able to neutralize multiple serovars, among them the most prevalent D, E and F (FIG. 2). Peptides representing the VD4 region or the conserved epitope derived from this region have been used for immunization either alone, as chimeric peptides fused to other regions such as VD1 or mixed with T cell epitopes to potentiate the antibody response ^{60, 68 51, 65 64 69}. All these constructs generated antibodies with some functional capabilities of neutralizing the infection in vitro but in general these strategies suffer from a low immunogenicity and the titres did not translate into in vivo protective efficacy against genital chlamydial challenge.

Reasons for the lack of protection when using these peptide based constructs can be numerous; including route of administration, type of immune response elicited, challenge dose, but most likely reflects that the vaccine molecule is not sufficiently immunogenic for use as a vaccine. The VD4 based strategy furthermore suffers from the limitation that with the exception of the TTLNPTIAG (SEQ ID NO: 76) epitope, these fragments as mentioned above are highly specific for one or two serovariants and a vaccine would accordingly have to be composed of several components to cover the most frequent serovariants causing human disease.

In WO2012172042 it has previously been disclosed that B-cell epitopes within the VD regions, combined with defined T cell (Th1 and Th2) epitopes from non-variable domains of MOMP, could function as a poly-epitope vaccine against Chlamydia psitattci serovar D in chickens; in the examples they describe the combination of up to three B-cell epitopes each derived from a VD region from different variable domains of the same serovariant together with several T-cell epitopes. The use of repeats of a variable domain of a surface exposed region of MOMP and using different serovariants is not suggested and thus high titers and a broad response against different serovariants is not obtained.

The object of the current invention is to prepare recombinant fusion molecules that are capable of generating a high titered neutralizing antibody response that is protective against various Ct serovars in vivo. Our invention furthermore describes the combination of these antibody promoting fragments with Ct antigens that are targets for T cells with the aim to provide a vaccine that activate both arms of the immune system.

### SUMMARY OF THE INVENTION

The present invention discloses an efficient vaccine against a pathogen, e.g. Chlamydia trachomatis (Ct), that incorporates repeats of surface exposed fragments of Ct antigens (homologous immuno-repeats) for maximal antibody responses. In one embodiment of the invention, these surface exposed fragments are extended to cover the flanking region of the surface exposed fragments that may contain T cell epitopes. One example is a defined large fragment representing an extended version of the VD1 or VD4 region from the Ct MOMP antigen and in the immuno-repeat format provides high levels of surface binding and neutralizing antibodies against Ct. In another important embodiment the immuno-repeat technology is used to obtain high titers and a broad response against different serovariants by the fusion of fragments that contain variable B and T cell epitopes from different serovariants (heterologous immuno-repeats). In yet another embodiment of our invention these surface exposed repeats are recombinantly fused with fragments of other surface exposed antigens such as PMPs or OMPs. Finally our invention discloses combinations of these immuno-repeat constructs with strong T cell antigens, such as MOMP (CT681), CT043 or CT004 from Ct that together form a very efficient vaccine against the different infectious stages of Ct infection.

### BRIEF DESCRIPTION OF THE SEVERAL VIEWS OF THE DRAWINGS

FIG. 1. Model of MOMP (Serovar D, strain: D/B-120) membrane topology adapted from Findlay et al ⁷⁷. The VD1, VD2, VD3 and VD4 are marked by black lines in the AA sequence (SEQ ID NO: 68) and in the linear model MOMP depicted interspaced with 5 constant segments (CS).

FIG. 2. Alignment of amino acid sequence of Ct MOMP VD4^{ext} for serovars D, E, F, G, Ia and J. The serovar D sequence is used as prototype, and conserved amino acids in other serovars are shown as “.”. The variable domain VD4 according to Baehr et al (PNAS, 1988) 36 is shaded in gray and the conserved epitope TTLNPTIAG (SEQ ID NO: 76) is boxed. S vD VD4^{ext} (SEQ ID NO: 23), S vE VD4^{ext} (SEQ ID NO: 24), S vF VD4^{ext} (SEQ ID NO: 25), S vG VD4^{ext} (SEQ ID NO: 26), S vI VD4^{ext} (SEQ ID NO: 27), and S vJ VD4^{ext} (SEQ ID NO: 26) are shown.

FIG. 3. Model of MOMP (Serovar D, strain: D/B-120) membrane topology adapted from Findlay et al. The VD1ext and VD4ext described in this invention are shown as shaded in the figure. Amino acid sequence shown is SEQ ID NO: 68.

FIG. 4. Illustration of the design of homologous and heterologous immuno-repeats. The immuno-repeats are fusion proteins of e.g. four VD4^{ext} regions, either from the same serovar, homologous immuno-repeats, or from different serovars, heterologous immuno-repeats. The variable VD4 region within each VD4^{ext} region is illustrated as hatched.

FIGS. 5A-5C. Enhanced and broadened immune responses after immunization with homologous immuno-repeats of VD4^{ext} compared with a monomeric VD4^{ext} unit.

FIG. 6. A construct composed of heterologous immuno-repeats from S vD, E, F and G induced a stronger response to multiple serovars compared to homologous immuno-repeats from S vF.

FIGS. 7A, 7B, 7C-A, 7C-A1, 7C-B, 7C-B1, 7C-C, 7C-C1, 7D-A, 7D-B, 7D-C. Fine specificity of the antibody responses after immunization with a heterologous immuno-repeat of the extended VD4 units from S vD, E, and F (CTH89) compared to constructs composed of a homologous immuno-repeat from (S vE^{ext}VD4)*4 and from (S vF^{ext.}VD4)*4. In FIGS. 7A-7B, the Serovar E sequence shown is SEQ ID NO: 24, and the Serovar F sequence shown is SEQ ID NO: 25. In FIGS. 7C-A through 7D-C, each set of overlapping peptides is NMFTPYIGV through MQIVSLQLN, corresponding to SEQ ID NO: 195 through SEQ ID NO: 254, respectively. (See Example 3).

FIGS. 8A-8F. Immunization with heterologous immuno-repeats of extended VD4's from S vD, S vE and S vF (CTH89) generates early T cell independent protection after a S vD challenge.

FIG. 9. In vivo neutralization with CTH89 specific serum.

FIGS. 10A-10D. Coupling of heterologous immuno-repeats to recombinant MOMP.

FIGS. 11A-11C. Vaccination with heterologous immuno-repeats of VD1-VD4's regions from S vD, S vE and S vF (CTH88) compared to vaccination with a single VD1-VD4 unit from S vD (CTH87)

FIGS. 12A-12D. Coupling of T cell antigens to immuno-repeats of VD4

FIGS. 13A-13E. Immunization with a cocktail of a heterologous VD4 immuno-repeat and a T cell antigen fusion molecule

FIGS. 14A-14B. Comparison of CAF01 and Alum as adjuvant delivery system.

FIGS. 15A-15C. Vaccination with heterologous immuno-repeats composed of reduced length of the VD4^{ext} regions from S vD, S vE, S vF and S vG. The CTH518 sequence shown is amino acids 1 through 68 of SEQ ID NO 53. The CTH286 sequence shown is amino acids 21 through 64 of SEQ ID NO 53. The CTH285 sequence shown is amino acids 28 through 57 of SEQ ID NO: 53.

FIGS. 16A-16D. Vaccination with heterologous immuno-repeats composed of extended VD4^{ext} regions from S vD, S vE, S vF, S vG, S vIa and S vJ. The CTH88 sequence shown is amino acids 60 through 127 of SEQ ID NO: 46. The CTH69+CTH72 sequence shown is SEQ ID NO: 255.

### DETAILED DISCLOSURE OF THE INVENTION

The invention discloses a polypeptide comprising
a) an amino acid sequence comprising one or more surface exposed fragments of the same outer membrane protein expressed in a serotype of Chlamydia sp.; and
b) two or more additional amino acid sequences which is either the same sequence as defined in a) or is the corresponding surface exposed fragments from a variant of said outer membrane protein expressed in a serotype of Chlamydia sp., which is different from the serotype in a).

The invention thus discloses polypeptides comprising immuno-repeats, which is 3 or more such as 4 or more repeats of an amino acid sequence comprising an immunogenic portion of a surface exposed region of an outer membrane protein of Chlamydia sp. Hence the invention can be described as a polypeptide comprising an amino acid sequence comprising one or more surface expose fragments of the same outer membrane protein expressed in a serotype of Chlamydia sp. and two or more such as three or more additional amino acid sequences which is either the same sequence as defined in a) or is the corresponding surface exposed fragments from a variant of said outer membrane protein expressed in a serotype of Chlamydia sp., which is different from the serotype in a).

In a preferred embodiment the polypeptide comprises 3 or more different amino acid sequences, where said amino acid sequences each comprises one or more surface exposed fragments from different variants or isotypes of the same outer membrane protein that varies in different Chlamydia sp. serotypes, said amino acid sequences derived from different Chlamydia sp. serotypes (heterologous immuno-repeats in our terminology), but the invention also discloses a polypeptide comprising 3 or more repetitions of an amino acid sequence, where said amino acid sequence comprises one or more surface exposed fragments of the same outer membrane protein that varies in different Chlamydia sp. serotypes, said amino acid sequences derived from the same Chlamydia sp. serotype (homologous immuno-repeats in our terminology).

The outer membrane protein is preferable the major outer membrane protein (MOMP) from any Chlamydia sp. serotype and the surface exposed fragment is chosen from variable domain 1 (VD1), variable domain 2 (VD2), variable domain 3 (VD3) or variable domain 4 (VD4) of MOMP. The surface exposed fragment can optionally be linearized by substitution of cysteine in the amino acid sequence to prevent disulfide bonds.

A preferred embodiment of the invention is polypeptides comprising immuno-repeats with 3 or more repeats of the variable domain 4 (VD4) of MOMP from any of serovars D, E, F, G, Ia and J of Chlamydia trachomatis, where each variable domain consists of an amino acid sequence, which corresponds to the position of amino acid residues Nos. 309-338 in the amino acid sequence of MOMP of Chlamydia trachomatis serovar D (S vD) (SEQ ID NO: 68) and where the variable domains in the immune-repeat is independently selected from the group consisting of the VD4 of serovar D, the VD4 of serovar E, the VD4 of serovar F, the VD4 of serovar G, the VD4 of serovar Ia and the VD4 of serovar J of Chlamydia trachomatis or has 80% sequence identity herewith.

The amino acid sequence of VD4 from serovar D, E, F, G, Ia and J corresponds to SEQ ID NO: 15-20 respectively. Each variable domain can additionally be flanked/extended on the N-terminal side by either
i) The amino acid sequence EWQASLALSYRLNMFTPYIGVKWSRASFDADTIRIAQPK (SEQ ID NO: 21) or
ii) A subsequence of the amino acid sequence in i) said subsequence comprising 1 or more amino acid residues,

On the C-terminal side the variable domain can additionally be flanked/extended by
iii) The amino acid sequence DTMQIVSLQLNKMKSRKSCGIAVGTTIVDA (SEQ ID NO: 22)
iv) A subsequence of the amino acid sequence in iv) said subsequence comprising 1 or more amino acid residues, or an amino acid sequence which has at least 80% sequence identity herewith.

Hence the preferred embodiment can be described as polypeptides comprising 2-8 different amino acid sequences each derived from MOMP from Chlamydia trachomatis which comprises an amino acid sequence defined in formula I:

xx₁-VD4-xx₂ (Formula I)

wherein
VD4 is independently selected from SEQ ID NO: 15-20 or an amino acid sequence which has at least 80% sequence identity herewith, and
xx₁ consists of
i) The amino acid sequence EWQASLALSYRLNMFTPYIGVKWSRASFDADTIRIAQPK (SEQ ID NO: 21) or
ii) A subsequence of the amino acid sequence in i) said subsequence comprising 1-38 amino acid residues, starting with the C-terminal K in the amino acid sequence in i) and
xx₂ consists of
iii) The amino acid sequence DTMQIVSLQLNKMKSRKSCGIAVGTTIVDA (SEQ ID NO: 22)
v) A subsequence of the amino acid sequence in iii) said subsequence comprising 1-29 amino acid residues, starting with the N-terminal D in the amino acid sequence in iii).

Examples of fusion proteins comprising immuno-repeats of VD4 of MOMP is indicated by SEQ ID NO: 49-59.

In another embodiment of the invention the polypeptide additionally comprises immuno-repeats of 3 or more variable domain 1 (VD1) of MOMP from any of serovars D, E, F, G, Ia and J of Chlamydia trachomatis, each variable domain consisting of an amino acid sequence, which corresponds to position of amino acid residues nos. 91-105 in the amino acid sequence of MOMP of Chlamydia trachomatis serovar D (S vD) (SEQ ID NO: 68) and is independently selected from the group consisting of the VD1 of serovar D, the VD1 of serovar E, the VD1 of serovar F, the VD1 of serovar G, the VD1 of serovar Ia and the VD1 of serovar J of Chlamydia trachomatis or has 80% sequence identity herewith.

The amino acid sequence of VD1 from serovar D, E, F, G, Ia and J corresponds to SEQ ID NO: 1-6 respectively. Each variable domain can additionally be flanked/extended on the N-terminal side by either
vi) The amino acid sequence SMRVGYYGDFVFDRVLKTDVNKEFQMG (SEQ ID NO: 77)
vii) A subsequence of the amino acid sequence in v) said subsequence comprising 1 or more amino acid residues.

On the C-terminal side the variable domain can additionally be flanked/extended by viii) The amino acid sequence NPAYGRHMQDAEMFTNAACMALNIWD (SEQ ID NO: 78)
ix) A subsequence of the amino acid sequence in x) said subsequence comprising 1 or more amino acid residues;

Or an amino acid sequence which has at least 80% sequence identity herewith.

Hence another preferred embodiment can be described as polypeptides comprising 2-8 different amino acid sequences each derived from MOMP from Chlamydia trachomatis which comprises an amino acid sequence defined in formula I and additionally comprising an amino acid sequence defined in formula II:

yy₁-VD1-yy₂ (Formula II)
wherein
VD1 is independently selected from SEQ ID NO: 1-6 or an amino acid sequence which has at least 80% sequence identity herewith,
and
yy₁ consists of
v) The amino acid sequence DAISMRVGYYGDFVFDRVLKTDVNKEFQMG (SEQ ID NO: 7) or
vi) A subsequence of the amino acid sequence in v) said subsequence comprising 1-30 amino acid residues, starting with the C-terminal G in the amino acid sequence in v)
and
yy₂ consists of
vii) The amino acid sequence NPAYGRHMQDAEMFTNAA (SEQ ID NO: 8) or
viii) A subsequence of the amino acid sequence in vii) said subsequence comprising 1-18 amino acid residues, starting with the N-terminal N in the amino acid sequence in vii).

Examples of polypeptides comprising immuno-repeats of VD1 is indicated by SEQ ID NO: 9-14 and 45-48.

Further embodiments of the invention comprises additionally comprises a fragment comprising the variable domains 2 (VD2) and/or variable domains 3 (VD3) of MOMP respectively comprising an amino acid sequence defined in formula III and/or formula IV:

zz₁-VD2-zz₂ (Formula III)

qq1-VD3-qq2 (Formula IV)
wherein
VD2 is independently selected from SEQ ID NO: 29-34 or an amino acid sequence which has at least 80% sequence identity herewith,
and
zz₁ consists of
ix) The amino acid sequence TLGATSGYLKGNSASFNLVGLFG (SEQ ID NO: 35) or
x) A subsequence of the amino acid sequence in ix) said subsequence comprising 1-23 amino acid residues, starting with the C-terminal G in the amino acid sequence in ix)
and
zz₂ consists of
xi) The amino acid sequence VVELYTDTTFAWSVGARAALWE (SEQ ID NO: 36) or
xii) A subsequence of the amino acid sequence in xi) said subsequence comprising 1-22 amino acid residues, starting with the N-terminal V in the amino acid sequence in xi).

And wherein wherein
VD3 is independently selected from SEQ ID NO: 37-42 or an amino acid sequence which has at least 80% sequence identity herewith,
and
qq₁ consists of
xiii) The amino acid sequence ATLGASFQYAQSKPKVEELNVLCNAAEFTINKPKGYVG (SEQ ID NO: 43) or
xiv) A subsequence of the amino acid sequence in xiii) said subsequence comprising 1-22 amino acid residues, starting with the C-terminal G in the amino acid sequence in xiii) and
qq₂ consists of
xv) The amino acid sequence TGTKDASIDYHEWQASLALSYRLNMFTPYIGVKWS (SEQ ID NO: 44) or
xvi) A subsequence of the amino acid sequence in xv) said subsequence comprising 1-35 amino acid residues, starting with the N-terminal T in the amino acid sequence in xv).

The immuno-repeats can be heterologous, that is where the variable domain is derived from different serotypes or they can be homologous, that is where the variable domain is derived one serotype. The preferred number of repeats are 2, 3, 4, 5, 6, 7 or 8 repeats.

Furthermore the immuno-repeats in the polypeptides can be linearized, that is cysteine residues are replaced with serine.

The polypeptides comprising immuno-repeats can additionally comprise a moiety that facilitate export of the polypeptide whens produced recombinantly (e.g. signal peptides), a moiety that facilitate purification of the polypeptide (e.g. his-tags) and/or a moiety which enhance the immunogenicity (e.g. a T cell antigen). The T-cell target can be chosen from a Ct antigen such as CT043, CT004, CT414, CT681 or part hereof. Examples of such fusion proteins are indicated by SEQ ID NO 60-67.

A polypeptide according to the invention having the following functional abilities:
a) neutralize C. trachomatis serovar D in vitro with a 50% neutralization titer of 10⁻³ or less, when tested in an experimental set-up comprising the administering a heterologous immuno-repeats;
b) neutralize C. trachomatis serovar D in vivo in at least 50% of the mice at day 7 post infection when tested in a mouse model comprising administering a heterologous immuno-repeats
c) broaden the immune response to multiple serovars of C. trachomatis in vitro when administering heterologous immuno-repeats.

The present invention also discloses nucleic acids encoding above described polypeptides.

The disclosed polypeptides or nucleic acids are used for the preparation of a pharmaceutical composition such as a vaccine. The vaccine can additionally comprise a pharmacologically acceptable carrier (virus like particles), excipient, adjuvant (e.g. DDA/TDB or alum) or immune modulator. The pharmaceutical composition can be used for prophylactic or therapeutic use against Chlamydia sp. Infections, including infections with Chlamydia trachomatis or C. pneumoniae.

A method for preventing, treating and/or reducing the incidence of Chlamydia sp. Infections, including infections with Chlamydia trachomatis or C. pneumoniae, by administering this pharmaceutical composition is also disclosed.

In the following the invention will be described in more detail and exemplified.

The preferred outer membrane protein is MOMP but may also include other surface exposed antigens from Chlamydia species that are targets for humoral responses.

The immuno-repeat from a surface exposed region can be from the same serotype (homologous immuno-repeats) or represent fragments that contain variable epitopes and are derived from different serotypes (heterologous immuno-repeat). In a preferred embodiment the immuno-repeats contain an extended fragment that contains both a variable and a conserved region known to be rich in T cell epitopes.

A preferred surface exposed region of an outer membrane protein is chosen from VD1, VD2, VD3 and VD4 from MOMP.

The amino acid sequences used for constructing the immuno-repeats described in the examples are chosen from table 1, 2 and 3.

The variable domain of VD4 of MOMP can be described as an amino acid sequences as defined as:

La1-Aa2-Aa1-Aa3-La2
wherein
Aa1 consists of the amino acid sequence TTLNPTIAG (SEQ ID NO: 76) (which is conserved for all serovars);
Aa2 is selected from the group consisting of: SATAIFDT (SEQ ID NO: 79) (from serovar D and E), LVTPVVDI (SEQ ID NO: 80) (from serovar F), LAKPVVDI (SEQ ID NO: 81) (from serovar G) and LAEAILDV (SEQ ID NO: 82) (from serovar Ia and J).

When Aa2 is the sequence from serovar D or E, then Aa3 is selected from the sequences set forth in AGDVKTGAEGQLG (SEQ ID NO: 83) (from serovar D) and AGDVKASAEGQLG (SEQ ID NO: 84) (serovar E).

When Aa2 is the sequence from serovar F, then Aa3 is the sequence CGSVAGANTEGQIS (SEQ ID NO: 85) (from serovar F).

When Aa2 is the sequence from serovar G, then Aa3 is the sequence CGSVVAANSEGQIS (SEQ ID NO: 86) (from serovar G).

When Aa2 is the sequence from serovar Ia or J), then Aa3 is selected from KGTVVSSAENELA (SEQ ID NO: 87) (from serovar Ia) and KGTVVASGSENDLA (SEQ ID NO: 88) (from serovar J)

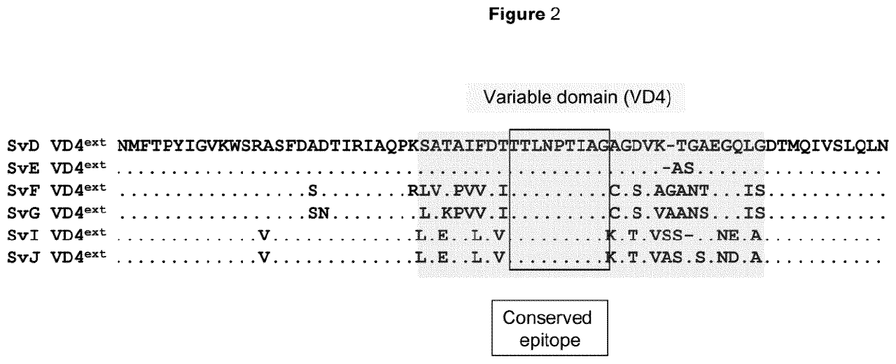

The variable domain VD4 of MOMP is depicted in FIG. 2. The immuno-repeats preferably additionally comprises extensions on either sides which are also depicted in FIG. 2.

The N-terminal side of a VD4 domain can be flanked or extended by one or more amino acids from the more conserved and T-cell epitope rich La1, where La1 is the part of VD4 of MOMP which is embedded in the membrane and has the amino acid sequence EWQASLALSYRLNMFTPYIGVKWSRASFDADTIRIAQPK (SEQ ID NO: 21) or an amino acid sequence having 80% sequence identity herewith.

The C-terminal side of a VD4 domain can correspondingly be flanked or extended by one or more amino acids from the more conserved and T-cell epitope rich La2, where La2 is the part of VD4 of MOMP which is embedded in the membrane on the C-terminal side and has the amino acid sequence DTMQIVSLQLNKMKSRKSCGIAVGTTIVDA (SEQ ID NO: 22) or an amino acid sequence having 80% sequence identity herewith.

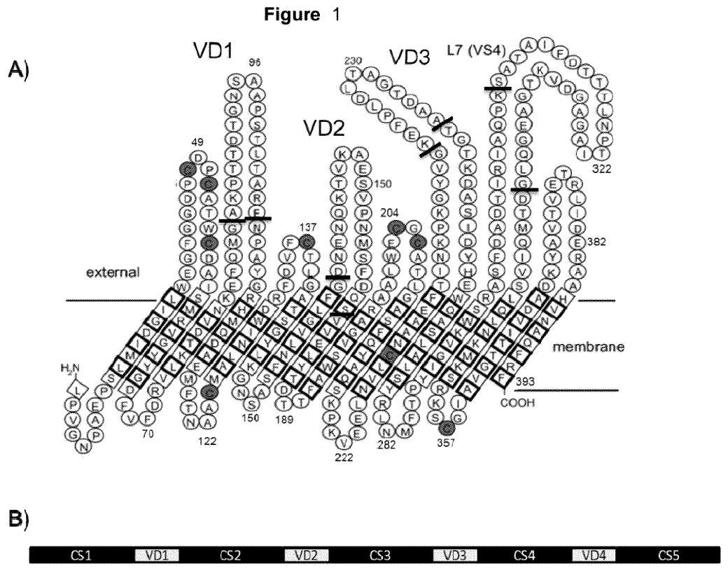

A similar illustration (see FIG. 1) can describe immuno-repeats comprising the variable domain 1 (VD1) of MOMP with the variable domains (Aa2-Aa1-Aa3) of the various serovars are given by SEQ ID NO: 1-6 in table 1. The corresponding N-terminal and C-terminal extensions (La1 and La2) have the respective amino acid sequences SMRVGYYGDFVFDRVLKTDVNKEFQMG (SEQ ID NO: 77) (La1) and NPAYGRHMQDAEMFTNAACMALNIWD (SEQ ID NO: 78) (La2) which are given in table 2 by SEQ ID NO: 7-8.

Immuno-repeats comprising VD2 and VD3 can in a similar manner be deduced from FIG. 1 and table 1.

Hence above example Lal-Aa2-Aa1-Aa3-La2 defines one of the immune-repeat units. If additionally e.g. VD1 is added to a VD4 unit, this can be described as adding one more sequence to make up a larger immune-repeat unit. Hence the polypeptide of the invention comprises 2, 3, 4, 5, 6, 7 or 8 repeats of immune-repeat units.

### Definitions

### Outer Membrane Proteins

The outer membrane of Chlamydia sp. can be isolated by treating intact, purified elementary bodies with detergent such as 2% Sarkosyl followed by ultracentrigation (100,000 g for one hour) which will lead to a supernatant with cytosolic components and a pellet containing the outer membrane as previously described 70. Outer membrane proteins can then be identified by standard protein techniques, e.g. by mass spectrometry after SDS-PAGE.

### Surface Exposed Fragments or Regions

Bacterial surface or membrane proteins comprises trans membrane proteins, secretory and lipoproteins, and anchorless surface proteins. Surface exposed regions on intact bacteria are accessible to antibodies. Methods to identify surface exposed regions of proteins (the ‘surfaceome’ comprise e.g. biotinylation of the membrane proteins in intact bacteria, followed by isolation of the biotin-labelled fraction using streptavidin. The isolated proteins can then be identified by mass spectrometry. Another approach is to treat intact bacteria with a protease, e.g. trypsin (‘shaving’) to cleave surface exposed peptides, followed by collection of the released peptides for identification by mass spectrometry.

### Variants

Variants of outer membrane proteins provided herein describes proteins encoded by the same gene from different serotypes of Chlamydia sp. A variant protein shares significant homology with a reference polypeptide.

### An Isoform of Protein

In the context of the present application an “isoform” of protein is understood as any of several different forms of the same protein e.g. a protein that has the same function but which is encoded by a different gene and may have small differences in its sequence or arises from either single nucleotide polymorphisms, differential splicing of mR NA, or post-translational modifications. Different serotypes of bacteria may have different isoforms of certain proteins.

### Chlamydia Species

By the term “Chlamydia species” is understood a bacterium capable of causing the Chlamydia infection in an animal or in a human being. Examples are C. trachomatis, C. pneumoniae and C. muridarum. Also in animals, several infections with Chlamydia sp. are known, e.g. Chlamydia suis infecting pigs, and Chlamydiaphila abortus which causes abortion in small ruminants (sheep and goats).

### Serovariants, Serovars or Serotypes

Based on the reactivity of specific mono clonal antibodies against and detailed sequence analysis of the MOMP variable regions Ct can be divided into 15 different serovariants and of these serovariants A, B, Ba and C causes Trachoma, D-K causes sexually transmitted disease (STD), L1-L3 causes Lymphogranuloma venerum, and MoPn (C. muridarum) infects mice. Serovariants are sometimes mentioned as serovars or serotypes with the same meaning.

### Immuno-Repeats

By immuno-repeats is understood: repetitive units of one or more amino acid sequences comprising an immunogenic portion or fragment of an antigen. The units that are repeated can be described as one or more VD regions, that optionally can be extended as described above, that are repeated e.g. 4 examples with three repeats VD4-VD4-VD4, VD4-VD1-VD4-VD1-VD4-VD1, VD4_{D}-VD4_{D}-VD4_{D}, VD4_{D}-VD4_{F}-VD4_{G}, VD4_{D}-VD3_{E}-VD4_{D}-VD3_{E}. VD4_{D}-VD3_{E}.

### Homologous Immuno-Repeat

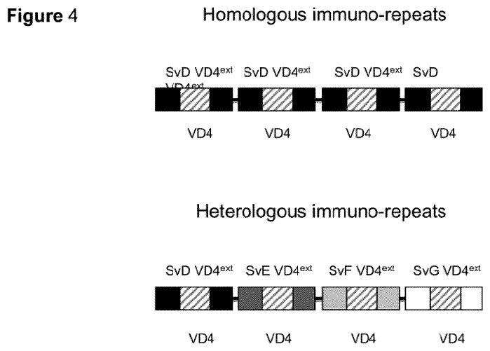

Repetitive units of one or more amino acid sequences comprising an immunogenic portion or fragment of an antigen from one serovariant only (FIG. 4)

### Heterologous Immuno-Repeat

Repetitive units of one or more amino acid sequences comprising an immunogenic portion or fragment encoding the same antigen derived from different serovariants (FIG. 4).

### Heterologous Challenge

Refers to the situation where the protein used for vaccination is derived from a different bacterial serovariant than the serovariant used for challenge.

### Homologous Challenge

Refers to the situation where the protein used for vaccination is derived from the same bacterial serovariant as the serovariant used for challenge.

### MOMP

The Major Outer Membrane Protein (MOMP) of Ct, is expressed during all phases of the developmental life cycle of Ct and constitutes approximately 60% of the total protein content of the chlamydia outer membrane. MOMP can be divided into conserved domains interrupted by four highly variable domains (VD1-4 or VS1-4)⁵⁹ (FIG. 1)

### VD1

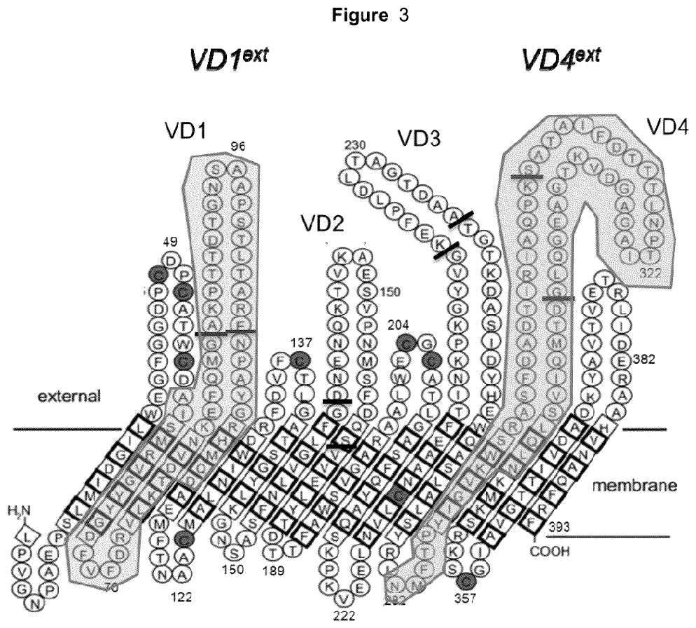

Variable domain 1 (VD1) of MOMP as defined by Baehr et al (1988)³⁶ which corresponds to amino acids 91-105 and make up a highly variable region in MOMP from Ct (Seq no 1-6 VD1 from S vD, E, F, G, Ia and J respectively). The extended VD1 region (VD1^{ext}) corresponds to amino acids 57-115 and make-up said highly variable region flanked by highly conserved regions in MOMP from Ct (Seq no 9-14 VD1^{ext} from S vD, E, F, G, Ia and J respectively) (FIG. 3).

### VD4

Variable domain 4 of MOMP as defined by Baehr et al (1988)³⁶ which corresponds to amino acids 309-338 and make up a highly variable region in MOMP from Ct (Seq no 15-20 VD4 from S vD, E, F, G, Ia and J respectively). The extended VD4 region (VD4^{ext}) corresponds to amino acids 282-349 and make-up said highly variable region flanked by highly conserved regions in MOMP from Ct (Seq no 23-28 VD4^{ext} from S vD, E F, G, Ia and J respectively).

### Linearized

The word “linearized” in the present invention refers to an amino acid chain of any length, including a full-length protein, oligopeptides, short peptides and fragments thereof, wherein the amino acid cysteine has been substituted with serine in order to hinder the cysteine residues to form disulfide bonds.

### Neutralizing Epitope

Neutralizing epitope as used herein is intended an amino acid sequence that defines an antigenic determinant which is bound by an antibody and, in the context of infection, reduces infectivity of a Chlamydial load, e.g. by blocking of the bacterial interaction with host cells, which is important in establishing bacterial infection and disease, facilitating bacterial clearance.

### Neutralization

Neutralization is to encompass any biological activity of the bacteria, including reduction in the efficiency or ability of the bacterium to establish infection or cause disease or disease symptoms, inhibition of chlamydial E B formation.

### Neutralizing Antibodies

Antibodies which bind a neutralizing epitope as described above.

### Polypeptides

The word “polypeptide” in the present invention should have its usual meaning. That is an amino acid chain of any length, including a full-length protein, oligopeptides, short peptides and fragments thereof, wherein the amino acid residues are linked by covalent peptide bonds.

### IFN-γ

By the term “IFN-γ” is understood interferon-gamma. The measurement of IFN-γ is used as an indication of an immunological T-cell response.

### Comprise

Throughout this specification, unless the context requires otherwise, the word “comprise”, or variations thereof such as “comprises” or “comprising”, will be understood to imply the inclusion of a stated element or integer or group of elements or integers but not the exclusion of any other element or integer or group of elements or integers.

### Immunogenic Portion or Fragment

In a preferred embodiment of the invention, the polypeptide comprises an immunogenic portion or fragment of the polypeptide, such as an epitope for a B-cell or T-cell.

The immunogenic portion or fragment of a polypeptide is a part of the polypeptide, which elicits an immune response in an animal or a human being, and/or in a biological sample determined by any of the biological assays described herein. The immunogenic portion or fragment of a polypeptide may be a T-cell epitope or a B-cell epitope. Immunogenic portions or fragments can be related to one or a few relatively small parts of the polypeptide, they can be scattered throughout the polypeptide sequence or be situated in specific parts of the polypeptide. For a few polypeptides epitopes have even been demonstrated to be scattered throughout the polypeptide covering the full sequence⁷¹.

In order to identify relevant T-cell epitopes which are recognised during an immune response, it is possible to use a “brute force” method: Since T-cell epitopes are linear, deletion mutants of the polypeptide will, if constructed systematically, reveal what regions of the polypeptide are essential in immune recognition, e.g. by subjecting these deletion mutants e.g. to the IFN-γ assay described herein. Another method utilises overlapping oligopeptides for the detection of MHC class II epitopes, preferably synthetic, having a length of e.g. 20 amino acid residues derived from the polypeptide. These peptides can be tested in biological assays (e.g. the IFN-γ assay as described herein) and some of these will give a positive response (and thereby be immunogenic) as evidence for the presence of a T cell epitope in the peptide. For the detection of MHC class I epitopes it is possible to predict peptides that will bind ⁷² and hereafter produce these peptides synthetic and test them in relevant biological assays e.g. the IFN-γ assay as described herein. The peptides preferably having a length of e.g. 8 to 11 amino acid residues derived from the polypeptide. B-cell epitopes can be determined by analysing the B cell recognition to overlapping peptides covering the polypeptide of interest as e.g. described in Harboe et al ⁷³.

### Immunogenic

An immunogenic polypeptide is defined as a polypeptide that induces an immune response in a biological sample or an individual currently or previously infected with a chlamydia.

### Fusion Proteins

By a fusion protein is understood two or more polypeptides linked together covalently. The fusion proteins can be produced with superior characteristics of the polypeptide. For instance, fusion partners that facilitate export of the fusion protein when produced recombinantly (e.g. signal peptides), fusion partners that facilitate purification of the fusion protein (e.g. his-tags), and fusion partners which enhance the immunogenicity of the fusion protein are all interesting possibilities. The fusion partner can, in order to enhance immunogenicity, be another polypeptide derived from C. trachomatis, such as a polypeptide, a polypeptide fragment or at least one T-cell epitope or B cell epitope.

### Pharmaceutical Composition

A pharmaceutical composition is defined as any vaccine (both therapeutic and prophylactic) or any diagnostic reagent.

### Vaccine, Protein

Another part of the invention pertains to a vaccine composition comprising a fusion protein or a nucleic acid encoding said fusion protein according to the invention. In order to ensure op-timum performance of such a vaccine composition it is preferred that it comprises an immunologically and pharmaceutically acceptable carrier, vehicle or adjuvant.

An effective vaccine, wherein a fusion protein of the invention is recognized by a mammal including a human being, will decrease bacterial load in target organs, prolong survival times and/or diminish weight loss after challenge with virulent chlamydial bacteria, compared to non-vaccinated individuals.

Suitable carriers are selected from the group consisting of a polymer to which the polypeptide(s) is/are bound by hydrophobic non-covalent interaction, such as a plastic, e.g. polystyrene, or a polymer to which the polypeptide(s) is/are covalently bound, such as a polysaccharide, or a polypeptide, e.g. bovine serum albumin, ovalbumin or keyhole limpet haemocyanin. Suitable vehicles are selected from the group consisting of a diluent and a suspending agent. The adjuvant is preferably selected from the group consisting of dimethyl-dioctadecylammonium bromide (DDA), Quil A, poly I:C, aluminium hydroxide, Freund's incomplete adjuvant, IF Nγ, IL-2, IL-12, monophosphoryl lipid A (MPL), Trehalose Dimycolate (TDM), Trehalose Dibephenate (TDB) and muramyl dipeptide (MDP), Monomycolyi glycerol (MMG) or a combination hereof. A preferred combination is a cationic liposome such as DDA combined with TDB and/or poly I:C.

Preparation of vaccines which contain peptide sequences as active ingredients is generally well understood in the art, as exemplified by U.S. Pat. Nos. 4,608,251; 4,601,903; 4,599,231 and 4,599,230, all incorporated herein by reference.

### Therapeutic Vaccine.

The invention also relates to the use of a polypeptide or nucleic acid of the invention for use as therapeutic vaccines as have been described in the literature exemplified by D. Lowry (Lowry et al 1999). Antigens with therapeutic properties may be identified based on their ability to diminish the severity of Ct infection in experimental animals or prevent reactivation of previous infection, when administered as a vaccine. The composition used for therapeutic vaccines can be prepared as described above for vaccines.

The present invention describes novel highly immunogenic vaccine antigens with broad antibody based neutralizing capacity that protects against different serovariants of Chlamydia trachomatis. We demonstrate that repetitive units of defined fragments from the MOMP antigen provide highly immunogenic molecules which we refer to as immuno-repeats. Vaccination with homologous immuno-repeats containing VD4 extended fragments (covers the VD4 variable domain of MOMP and the adjacent conserved flanking regions) in different adjuvants provides very high antibody titers and we demonstrate that these constructs are much more efficient than immunizing with single units of the VD4 extended fragment. The increased effect can be observed both as markedly increased titer, increased antibody targeting of the surface of the bacteria, increased neutralizing capacity, increased and broadened T cell response and increased protection against a challenge with the homologous strain. We furthermore demonstrate that the immuno-repeat technology can be utilized also to improve the protection against and neutralization of other serovariants by constructing heterologous immuno-repeats based on VD4 extended fragments from different serovariants such as serovar D, E, F and G (FIG. 3).

TABLE 1
Description of sequences used in constructing immuno-repeats

| SEQ | | |
|---|---|---|
| ID NO | Variable domaines | Description |
| 1 | VD1_SvD | Serovar D variable domaine 1 of MOMP |
| 2 | VD1_SvE | Serovar E variable domaine 1 of MOMP |
| 3 | VD1_SvF | Serovar F variable domaine 1 of MOMP |
| 4 | VD1_SvG | Serovar G variable domaine 1 of MOMP |
| 5 | VD1_SvIa | Serovar Ia variable domaine 1 of MOMP |
| 6 | VD1_SvJ | Serovar J variable domaine 1 of MOMP |
| 7 | VD1 N-terminal | VD1 N-terminal extension |
| 8 | VD1 C-terminal | VD1 C-terminal extension |
| 9 | VD1ext_SvD | Serovar D extended VD1 of MOMP |
| 10 | VD1ext_SvE | Serovar E extended VD1 of MOMP |
| 11 | VD1ext_SvF | Serovar F extended VD1 of MOMP |
| 12 | VD1ext_SvG | Serovar G extended VD1 of MOMP |
| 13 | VD1ext_SvIa | Serovar Ia extended VD1 of MOMP |
| 14 | VD1ext_SvJ | Serovar J extended VD1 of MOMP |
| 15 | VD4_SvD | Serovar D variable domaine 4 of MOMP |
| 16 | VD4_SvE | Serovar E variable domaine 4 of MOMP |
| 17 | VD4_SvF | Serovar F variable domaine 4 of MOMP |
| 18 | VD4_SvG | Serovar G variable domaine 4 of MOMP |
| 19 | VD4_SvIa | Serovar Ia variable domaine 4 of MOMP |
| 20 | VD4_SvJ | Serovar J variable domaine 4 of MOMP |
| 21 | VD4 N-terminal | VD4 N-terminal extension |
| 22 | VD4 C-terminal | VD4 C-terminal extension |
| 23 | VD4ext_SvD | Serovar D extended VD4 of MOMP |
| 24 | VD4ext_SvE | Serovar E extended VD4 of MOMP |
| 25 | VD4ext_SvF | Serovar F extended VD4 of MOMP |
| 26 | VD4ext_SvG | Serovar G extended VD4 of MOMP |
| 27 | VD4ext_SvIa | Serovar Ia extended VD4 of MOMP |
| 28 | VD4ext_SvJ | Serovar J extended VD4 of MOMP |
| 29 | VD2_SvD | Serovar D variable domaine 2 of MOMP |
| 30 | VD2_SvE | Serovar E variable domaine 2 of MOMP |
| 31 | VD2_SvF | Serovar F variable domaine 2 of MOMP |
| 32 | VD2_SvG | Serovar G variable domaine 2 of MOMP |
| 33 | VD2_SvIa | Serovar Ia variable domaine 2 of MOMP |
| 34 | VD2_SvJ | Serovar J variable domaine 2 of MOMP |
| 35 | VD2 N-terminal | VD2 N-terminal extension |
| 36 | VD2 C-terminal | VD2 C-terminal extension |
| 37 | VD3_SvD | Serovar D variable domaine 3 of MOMP |
| 38 | VD3_SvE | Serovar E variable domaine 3 of MOMP |
| 39 | VD3_SvF | Serovar F variable domaine 3 of MOMP |
| 40 | VD3_SvG | Serovar G variable domaine 3 of MOMP |
| 41 | VD3_SvIa | Serovar Ia variable domaine 3 of MOMP |
| 42 | VD3_SvJ | Serovar J variable domaine 3 of MOMP |
| 43 | VD3 N-terminal | VD3 N-terminal extension |
| 44 | VD3 C-terminal | VD3 C-terminal extension |

Heterologous immuno-repeats were highly immunogenic but in addition increased the breadth of the antibody responses which was associated with a broader fine specificity of the antibody response (measured by peptide scans) that targets a more diverse repertoire of linear epitopes within the VD4 region than the homologous immuno-repeats. We also demonstrate that highly immunogenic heterologous immuno-repeats can be based on even larger fragments that incorporate fusions of VD1 and VD4 extended fragments and we confirm that in animal models protection promoted by these heterologous immuno-repeats are mediated predominantly by antibodies. As there is a generally recognized need for a strong CMI component (e.g. a T-cell epitope) in an efficient protective immune response against Ct, we have also demonstrated that by fully extending the VD4 region N-terminally to include a T cell rich region, we can generate immune-repeats that combine the ability to generate high tittered neutralizing antibodies with a strong T cell response clearing residual infection in one construct. We have also demonstrated that immune-repeats can be fused to or mixed with T-cell antigens with vaccine potential and that this combination provide both an early antibody mediated protection against Ct as well as an efficient CMI mediated clearance of residual organisms.

MOMP is an important protective antigen with a generally recognized potential in Ct vaccines. The MOMP antigen is however a very complicated antigen to target by vaccines because it has a complex structure with numerous internal disulfide bonds and where important neutralizing epitopes have been exceedingly difficult to expose in recombinant molecules. Adding to this, the MOMP antigen is highly variable and is the basis for the majority of the serovariance found in different strains causing human disease. Any vaccine based on intact MOMP would therefore have to incorporate a number of different versions of the molecule (at least 4-5) to cover the major strains giving rise to disease in humans. As described above the MOMP antigen contains 4 variable regions (VD1-4) of which in particular the VD1 and VD4 contain important neutralizing epitopes but vaccines based on fragments representing these regions have so far failed to induce sufficiently high titers of functional antibodies to have any in vivo effect in animal challenge studies ^{51 74}.

The immuno-repeat technology of the present invention solves this problem: By repeating the important variable VD1 and/or VD4 regions flanked by conserved sequences from the MOMP antigen we have obtained immunogens that promote extraordinary levels of functional antibodies. Surprisingly we also demonstrate that the improved immunogenicity can even be achieved in heterologous immuno-repeat constructs that employs variable regions from different serovars interspaced between conserved fragments and that this strategy produces a broadly neutralizing antibody response that protect against different serovariants. Furthermore, do the immuno-repeat technology provide a large number of relevant T cell epitopes that promote T cells with direct effector function as well as the ability to promote accelerated recall responses to the adjacent B cell epitopes.

Our invention therefore represents a breakthrough in developing efficient Ct vaccines with a broad response and the ability to neutralize different serovars.

It is well known that antigens with a large number of repeats and organized structure are optimal for the activation of the B-cell receptor (BCR), leading to an increased humoral response and a decreased dependence on T-cell help. This was originally reported with natural polysaccharide based antigens from various pathogens (Pneumococcal polysaccharide and Salmonella polymerized flagellin) where the repetitive nature of the antigen is assumed to trigger several BCR simultaneously thereby lowering the overall activation threshold which triggers antibody production from plasma B-cells without the need for prior T-cell help. Such antigens are referred to as type 2 T-cell independent B-cell antigens and in artificial systems have been shown to depend on a large number of repeats (typically a minimum of 12-16 ⁷⁵), that constitute the minimal epitope and are closely located. This is clearly different from our repeat technology where large fragments (69 amino acids, Mw>7 kDa) are repeated and these fragments contain both B-cell and T-cell epitopes⁷⁶.

In contrast to previous observations ⁷⁵, we observe an increase by just 4 repeats which is not further improved by 8 repeats. Importantly, the repetition of a conserved sequence with hypervariable domains inserted, amplify responses not only to the repeated conserved element but importantly to the variable inserts. The molecular mechanism behind this surprising amplification is not completely clear but it most likely relates to the fact that many of the important epitopes are located in the overlap between variable and conserved regions which therefore may allow simultaneous triggering of different BCR's that all share some recognition of the conserved part of the epitope. Although the mechanism is not completely clear the practical consequence is that the heterologous immune-repeat technology allows the synthesis of a multivalent immunogens that promote the generation of a diverse antibody response that targets different serovariants.

Our immuno-repeat constructs provide antigens of an extraordinary immunogenicity compared to previous attempts to use the variable domains from Ct MOMP. All previous vaccines based on VDs of MOMP did, in spite of generating antibodies with some functional capabilities, fail to generate titres that translated into in vivo protection against genital chlamydial challenge ^{51, 65 64}. In particular the heterologous immuno-repeat strategy solves a very fundamental problem seen for many pathogens and that is how to promote diverse antibody responses to diverse and variable antigens.

TABLE 2
Immuno-repeats

| SEQ ID NO | Polypeptide names | Description |
|---|---|---|
| 45 | CTH87 (CT681_VD1ext_VD4ext_SvD) | Fusion of VD1-VD4 of serovar D |
| 46 | CTH88 (CT681_lin_VD1ext_VD4ext_SvD_E_F) | Heterologous immune repeat of VD1- |
| | | VD4 |
| 47 | CTH88ext = CTH69 | Same as SEQ ID NO 46 with longer |
| | (CT681_lin_VD1ext_VD4ext_SvD_E_F_ext) | flanking region. |
| 48 | CTH72 | Same as seq id no 47 additionally with |
| | (CT681_lin_VD1ext_VD4ext_SvD_E_F_G_Ia_J_ext) | VD1ext and VD4ext from SvG, SvIa and |
| | | SvJ |
| 49 | CTH89 (CT681_lin_VD4ext_SvD_E_F) | Heterologous immune repeat of VD4 |
| 50 | CTH181 (CT681_VD4ext_SvE) | Same as SEQ ID NO 24 |
| 51 | CTH182 (CT681_lin_VD4ext_F) | Same as SEQ ID NO 25 linearized |
| 52 | CTH183 (CT681_VD4ext_F) | Same as SEQ ID NO 25 |
| 53 | CTH518 (CT681_Lin_VD4ext_D_E_F_G) | Heterologous immune repeat of VD4 |
| 54 | CTH518ext = CTH70 | Same as SEQ ID NO 53 with longer |
| | (CT681_lin_VD4ext_SvD_E_F_G_ext) | flanking regions |
| 55 | CTH71 | Same as seq id no 54 additionally with |
| | (CT681_lin_VD4ext_SvD_E_F_G_Ia_J_ext) | VD1ext and VD4ext from SvIa and SvJ |
| 56 | CTH524 (CT681_lin_4_VD4ext_F) | Same as SEQ ID NO 59 linearized |
| 57 | CTH526 (CT681_8_VD4ext_SvE) | Homologous immune repeat of VD4 (8x) |
| 58 | CTH527 (CT681_4_VD4ext_SvE) | Homologous immune repeat of VD4 (4x) |
| 59 | CTH529 (CT681_4_VD4ext_F) | Homologous immune repeat of VD4 (4x) |

TABLE 3
Examples of immuno-repeats fused with T-cell antigens

| SEQ | |
|---|---|
| ID | Fusions of immuno repeats with T-cell antigens |
| NO | (all his-tagged) |
| 60 | CTH91 (CT043-CT414p- |
| | CT681_lin_VD1ext_VD4ext_SvD_E_F) |
| 61 | CTH93 (CT043_CT414p_CT681_Lin_56-281_VD4ext_D) |
| 62 | CTH520 (CT681_56-281_VD4ext_D) |
| 63 | CTH521 (CT681_Lin_56-281_VD4ext_D) |
| 64 | CTH522 (CT681_lin_56-281_VD4ext_D_E_F_G) |
| 65 | CTH531 (CT414_CT043_CT043_681_lin_56- |
| | 281_VD4ext_SvD_E_F_G) |
| 66 | CTH533 (CT043_CT043_CT681_lin_VD4ext_SvD_E_F_G) |
| 67 | CTH534 |
| | (CT043_CT043_CT004_CT681_lin_VD4ext_SvD_E_F_G) |
| 68 | CT681_SvD |
| 69 | CTH285 (VD4_lin_SvD, E, F, G) |
| 70 | CTH286 (VD4 classic + 7_lin_SvD, E, F, G) |

TABLE 4
Overlapping peptides of VD4 from serovar E

| VD4 serovar E | | SEQ ID |
|---|---|---|
| peptides (20mers) | Amino acid sequence | NO |
| CT681_25_SvE | DASIDYHEWQASLALSYRLN | 89 |
| CT681_26_SvE | ASLALSYRLNMFTPYIGVKW | 90 |
| CT681_27_SvE | MFTPYIGVKWSRASFDADTI | 91 |
| CT681_28_SvE | SRASFDADTIRIAQPKSATA | 92 |
| CT681_29_SvE | RIAQPKSATAIFDTTTLNPT | 93 |
| CT681_30_SvE | IFDTTTLNPTIAGAGDVKAS | 94 |
| CT681_31_SvE | IAGAGDVKASAEGQLGDTMQ | 95 |
| CT681_32_SvE | AEGQLGDTMQIVSLQLNKMK | 96 |

TABLE 5
Overlapping peptides of VD4 from serovar F

| Serovar F | | SEQ ID |
|---|---|---|
| peptides (20mers) | Amino acid sequence | NO |
| CT681_25_SvF | DASIDYHEWQASLSLSYRLN | 97 |
| CT681_26_SvF | ASLSLSYRLNMFTPYIGVKW | 98 |
| CT681_27_SvF | MFTPYIGVKWSRASFDSDTI | 99 |
| CT681_28_SvF | SRASFDSDTIRIAQPRLVTP | 100 |
| CT681_29_SvF | RIAQPRLVTPVVDITTLNPT | 101 |
| CT681_30_SvF | VVDITTLNPTIAGCGSVAGA | 102 |
| CT681_31_SvF | IAGCGSVAGANTEGQISDTMQ | 103 |
| CT681_32_SvF | TEGQISDTMQIVSLQLNKMK | 104 |

TABLE 6
Overlapping peptides of VD4 from serovar D

| VD4 serovar D | Amino acid | SEQ |
|---|---|---|
| peptides (9mers) | sequence | ID NO |
| VD4_P1_SvD | SRASFDADT | 105 |
| VD4_P2_SvD | RASFDADTI | 106 |
| VD4_P3_SvD | ASFDADTIR | 107 |
| VD4_P4_SvD | SFDADTIRI | 108 |
| VD4_P5_SvD | FDADTIRIA | 109 |
| VD4_P6_SvD | DADTIRIAQ | 110 |
| VD4_P7_SvD | ADTIRIAQP | 111 |
| VD4_P8_SvD | DTIRIAQPK | 112 |
| VD4_P9_SvD | TIRIAQPKS | 113 |
| VD4_P10_SvD | IRIAQPKSA | 114 |
| VD4_P11_SvD | RIAQPKSAT | 115 |
| VD4_P12_SvD | IAQPKSATA | 116 |
| VD4_P13_SvD | AQPKSATAI | 117 |
| VD4_P14_SvD | QPKSATAIF | 118 |
| VD4_P15_SvD | PKSATAIFD | 119 |
| VD4_P16_SvD | KSATAIFDT | 120 |
| VD4_P17_SvD | SATAIFDTT | 121 |
| VD4_P18_SvD | ATAIFDTTT | 122 |
| VD4_P19_SvD | TAIFDTTTL | 123 |
| VD4_P20_SvD | AIFDTTTLN | 124 |
| VD4_P21_SvD | IFDTTTLNP | 125 |
| VD4_P22_SvD | FDTTTLNPT | 126 |
| VD4_P23_SvD | DTTTLNPTI | 127 |
| VD4_P24_SvD | TTTLNPTIA | 128 |
| VD4_P25_SvD | TTLNPTIAG | 76 |
| VD4_P26_SvD | TLNPTIAGA | 129 |
| VD4_P27_SvD | LNPTIAGAG | 130 |
| VD4_P28_SvD | NPTIAGAGD | 131 |
| VD4_P29_SvD | PTIAGAGDV | 132 |
| VD4_P30_SvD | TIAGAGDVK | 133 |
| VD4_P31_SvD | IAGAGDVKT | 134 |
| VD4_P32_SvD | AGAGDVKTG | 135 |
| VD4_P33_SvD | GAGDVKTGA | 136 |
| VD4_P34_SvD | AGDVKTGAE | 137 |
| VD4_P35_SvD | GDVKTGAEG | 138 |
| VD4_P36_SvD | DVKTGAEGQ | 139 |
| VD4_P37_SvD | VKTGAEGQL | 140 |
| VD4_P38_SvD | KTGAEGQLG | 141 |
| VD4_P39_SvD | TGAEGQLGD | 142 |
| VD4_P40_SvD | GAEGQLGDT | 143 |
| VD4_P41_SvD | AEGQLGDTM | 144 |
| VD4_P42_SvD | EGQLGDTMQ | 145 |
| VD4_P43_SvD | GQLGDTMQI | 146 |
| VD4_P44_SvD | QLGDTMQIV | 147 |
| VD4_P45_SvD | LGDTMQIVS | 148 |

TABLE 7
Overlapping peptides of VD4 from serovar F

| VD4 serovar F | Amino acid | SEQ |
|---|---|---|
| peptides (9mers) | sequence | ID NO |
| VD4_P1_SvF | SRASFDSDT | 149 |
| VD4_P2_SvF | RASFDSDTI | 150 |
| VD4_P3_SvF | ASFDSDTIR | 151 |
| VD4_P4_SvF | SFDSDTIRI | 152 |
| VD4_P5_SvF | FDSDTIRIA | 153 |
| VD4_P6_SvF | DSDTIRIAQ | 154 |
| VD4_P7_SvF | SDTIRIAQP | 155 |
| VD4_P8_SvF | DTIRIAQPR | 156 |
| VD4_P9_SvF | TIRIAQPRL | 157 |
| VD4_P10_SvF | IRIAQPRLV | 158 |
| VD4_P11_SvF | RIAQPRLVT | 159 |
| VD4_P12_SvF | IAQPRLVTP | 160 |
| VD4_P13_SvF | AQPRLVTPV | 161 |
| VD4_P14_SvF | QPRLVTPVV | 162 |
| VD4_P15_SvF | PRLVTPVVD | 163 |
| VD4_P16_SvF | RLVTPVVDI | 164 |
| VD4_P17_SvF | LVTPVVDIT | 165 |
| VD4_P18_SvF | VTPVVDITT | 166 |
| VD4_P19_SvF | TPVVDITTL | 167 |
| VD4_P20_SvF | PVVDITTLN | 168 |
| VD4_P21_SvF | VVDITTLNP | 169 |
| VD4_P22_SvF | VDITTLNPT | 170 |
| VD4_P23_SvF | DITTLNPTI | 171 |
| VD4_P24_SvF | ITTLNPTIA | 172 |
| VD4_P25_SvF | TTLNPTIAG | 76 |
| VD4_P26_SvF | TLNPTIAGC | 173 |
| VD4_P27_SvF | LNPTIAGCG | 174 |
| VD4_P28_SvF | NPTIAGCGS | 175 |
| VD4_P29_SvF | PTIAGCGSV | 176 |
| VD4_P30_SvF | TIAGCGSVA | 177 |
| VD4_P31_SvF | IAGCGSVAG | 178 |
| VD4_P32_SvF | AGCGSVAGA | 179 |
| VD4_P33_SvF | GCGSVAGAN | 180 |
| VD4_P34_SvF | CGSVAGANT | 181 |
| VD4_P35_SvF | GSVAGANTE | 182 |
| VD4_P36_SvF | SVAGANTEG | 183 |
| VD4_P37_SvF | VAGANTEGQ | 184 |
| VD4_P38_SvF | AGANTEGQI | 185 |
| VD4_P39_SvF | GANTEGQIS | 186 |
| VD4_P40_SvF | ANTEGQISD | 187 |
| VD4_P41_SvF | NTEGQISDT | 188 |
| VD4_P42_SvF | TEGQISDTM | 189 |
| VD4_P43_SvF | EGQISDTMQ | 190 |
| VD4_P44_SvF | GQISDTMQI | 191 |
| VD4_P45_SvF | QISDTMQIV | 192 |
| VD4_P46_SvF | ISDTMQIVS | 193 |

TABLE 8
CT681 amino acid sequences

| | Amino acid sequences of MOMP |
|---|---|
| SEQ ID NO | (CT681) from different serovars |
| 68 | CT681_SvD |
| 71 | CT681_SvE |
| 72 | CT681_SvF |
| 73 | CT681_SvG |
| 74 | CT681_SvIa |
| 75 | CT681_SvJ |

The nucleic acid of the invention, that is nucleic acid encoding above mentioned fusion proteins, may be used for effecting in vivo expression of immunogenic polypeptides, i.e. the nucleic acid may be used in so-called DNA vaccines as reviewed in Ulmer et al 1993, which is included by reference.

In the construction and preparation of plasmid DNA encoding a fusion polypeptide to be used defined for DNA vaccination a host strain such as E. coli can be used. Plasmid DNA can then be prepared from overnight cultures of the host strain carrying the plasmid of interest, and purified using e.g. the Qiagen Giga-Plasmid column kit (Qiagen, Santa Clarita, CA, USA) including an endotoxin removal step. It is essential that plasmid DNA used for DNA vaccination is endotoxin free.

Hence, the invention also relates to a vaccine comprising a nucleic acid according to the invention, the vaccine effecting in vivo expression of the immunogenic polypeptide by an animal, including a human being, to whom the vaccine has been administered, the amount of expressed polypeptide being effective to confer substantially increased resistance to infections caused by virulent bacteria in an animal, including a human being.

The efficacy of such a DNA vaccine can possibly be enhanced by administering the gene encoding the expression product together with a DNA fragment encoding a polypeptide which has the capability of modulating an immune response.

One possibility for effectively activating a cellular immune response can be achieved by expressing the relevant immunogenic polypeptide in a non-pathogenic microorganism or virus. Well-known examples of such microorganisms are Mycobacterium bovis BCG, Salmonella and Pseudomona and examples of viruses are Vaccinia Virus and Adenovirus.

Therefore, another important aspect of the present invention is an improvement of the live BCG vaccine presently available, wherein one or more copies of a DNA sequence encoding one or more fusion polypeptides as defined above has been incorporated into the genome of the micro-organism in a manner allowing the micro-organism to express and secrete the fusion polypeptide. The incorporation of more than one copy of a nucleic acid sequence of the invention is contemplated to enhance the immune response.

Another possibility is to integrate the DNA encoding the fusion polypeptide according to the invention in an attenuated virus such as the Vaccinia virus or Adenovirus (Rolph et al 1997). The recombinant vaccinia virus is able to enter within the cytoplasma or nucleus of the infected host cell and the fusion polypeptide of interest can therefore induce an immune response, which is envisioned to induce protection against TB.

Although DNA vaccines were developed more than 16 years ago, clinical trials preceding stage I and II in humans are rare. Two veterinary DNA vaccines however, have been licensed; one for West Nile Virus (in horse) and a second for Infectious Hematopoetic Necrosis virus in Salmon. This demonstrates that DNA vaccines can have good protective effects and that new DNA vaccines are not limited by the size of the animal or species. The great success with DNA vaccines observed for the murine model for first generation DNA vaccines did not translate well to humans, nonetheless; researchers have recently demonstrated protective antibodies levels by a single dose of gene gun administrated HA DNA vaccine to humans.

“Nucleic acid immunization” or the commonly preferred name “DNA vaccines” are the inoculation of antigen encoding DNA or RNA as expression cassettes or expression vectors or incorporated into viral vectors with the purpose of inducing immunity to the gene product. Thus, in our definition of DNA vaccines we include all kinds of delivery systems for the antigen encoding DNA or RNA. The vaccine gene can be in form of circular plasmid or a linear expression cassette with just the key features necessary for expression (promotor, the vaccine gene and polyadenylation signal). Delivery systems may most often be naked DNA in buffer with or without adjuvant, DNA coupled to nanoparticles and/or formulated into adjuvant containing compounds or inserted into live viral or bacterial vectors such as Adenovirus, adeno associated virus, alphavirus, poxviruses, herpes virus etc. DNA vaccines hold great promise since they evoke both humoral and cell-mediated immunity, without the same dangers associated with live virus vaccines. In contrast to live attenuated virus vaccines DNA vaccines may be delivered to same or different tissue or cells than the live virus that has to bind to specific receptors. The production of antigens in their native forms improves the presentation of the antigens to the host immune system. Unlike live attenuated vaccines, DNA vaccines are not infectious and cannot revert to virulence.

DNA vaccines offer many advantages over conventional vaccines. It can be produced in high amounts in short time, abolishing the need for propagation in eggs, it is cost-effective, reproducible and the final product does not require cold storage conditions, because DNA is stable and resistant to the extremes of temperature. All currently licensed inactivated vaccines are efficient at inducing humoral antibody responses but only live attenuated virus vaccines efficiently induce a cytotoxic cellular response as well. DNA vaccines also have this ability and the induced response therefore may better mimic the natural response to viral infection than inactivated vaccines in respect to specificity and antibodies isotypes.

DNA vaccines induce an immune response which is comparable to the response acquired by natural virus infection by activating both humoral and cell-mediated immunity. The broad response to DNA vaccines is a result of the encoded genes being expressed by the transfected host cell, inducing both a Th1 and Th2 immune responses. The production of antigens in their native form improves the presentation of the antigens to the host immune system.

The two most common types of DNA vaccine administration are saline injection of naked DNA and gene gun DNA inoculations (DNA coated on solid gold beads administrated with helium pressure). Saline intra muscular injections of DNA preferentially generates a Th1 IgG2a response while gene gun delivery tends to initiate a more Th2 IgG1 response. Intramuscular injected plasmids are at risk of being degraded by extracellular deoxyribonucleases, however, the responses induced are often more long-lived than those induced by the gene gun method. Vaccination by gene gun delivery of DNA, to the epidermis, has proven to be the most effective method of immunization, probably because the skin contains all the necessary cells types, including professional antigen presenting cells (APC), for eliciting both humoral and cytotoxic cellular immune responses (Langerhans and dendritic cells). Complete protection from a lethal dose of influenza virus has been obtained with as little as 1 μg DNA in mice. The standard DNA vaccine vector consists of the gene of interest cloned into a bacterial plasmid engineered for optimal expression in eukaryotic cells. Essential features include; an origin of replication allowing for production in bacteria, a bacterial antibiotic resistance gene allowing for plasmid selection in bacterial culture, a strong constitutive promotor for optimal expression in mammalian cells (promoters derived from cytomegalovirus (CMV) or simian virus provide the highest gene expression), a polyadenylation sequence to stabilise the mR NA transcripts, such as bovine growth hormone (BHG) or simian virus polyadenylation, and a multiple cloning site for insertion of an antigen gene. An intron A sequence improves expression of genes remarkably. Many bacterial DNA vaccine vectors contain unmethylated cytidinephosphate-guanosine (CpG) dinucleotide motifs that may elicit strong innate immune responses in the host. In recent years there have been several approaches to enhance and customise the immune response to DNA vaccine constructs (2nd generation DNA vaccines). For instance dicistronic vectors or multiple geneexpressing plasmids have been used to express two genes simultaneously. Specific promoters have been engineered that restrict gene expression to certain tissues, and cytokine/antigen fusion genes have been constructed to enhance the immune response. Furthermore, genes may be codon optimised for optimal gene expression in the host and naïve leader sequences may be substituted with optimised leaders increasing translation efficiency.

The administration of DNA vaccine can be by saline or buffered saline injection of naked DNA or RNA, or injection of DNA plasmid or linear gene expressing DNA fragments coupled to particles, or inoculated by gene gun or delivered by a viral vector (virus like particle) such as Adenovirus, Modified vaccinia virus Ankara (MVA), Vaccinia, Adenoassociated virus (AAV), Alphavirus etc.

In one embodiment is a polypeptide comprising
a) an amino acid sequence comprising one or more surface exposed fragments of the same outer membrane protein expressed in a serotype of Chlamydia sp.; and
b) two or more additional amino acid sequences which is either the same sequence as defined in a) or is the corresponding surface exposed fragments from a variant of said outer membrane protein expressed in a serotype of Chlamydia sp., which is different from the serotype in a).

In a further embodiment is a polypeptide comprising 3 or more different amino acid sequences, where said amino acid sequences each comprises one or more surface exposed fragments from different variants of the same outer membrane protein that varies in different Chlamydia sp. serotypes, said amino acid sequences derived from different Chlamydia sp. serotypes.

In another further embodiment is a polypeptide comprising 3 or more repetitions of an amino acid sequence, where said amino acid sequence comprises one or more surface exposed fragments of the same outer membrane protein that varies in different Chlamydia sp. serotypes, said amino acid sequences derived from the same Chlamydia sp. serotype.

A polypeptide as described above is provided, wherein the outer membrane protein is MOMP from any serotype. The outer membrane protein may be MOMP from serotype D, E, F, G, Ia or J of Chlamydia trachomatis or C. pneumoniae. Still further, a polypeptide may comprise one or more of the variable domains 1, 2, 3, 4 of MOMP. These variable domain sequences may optionally be linearized. These variable domain sequences may comprise the variable domains 4 (VD4) of MOMP, and may be placed next to each other or be spaced with a linker. In an embodiment thereof is a polypeptide comprising an amino acid sequence defined in formula I:

xx₁-VD4-xx₂ (Formula I)
wherein
VD4 is independently selected from SEQ ID NO: 15-20 or an amino acid sequence which has at least 80% sequence identity herewith
and
xx₁ consists of
i) The amino acid sequence EWQASLALSYRLNMFTPYIGVKWSRASFDADTIRIAQPK (SEQ ID NO: 21) or
ii) A subsequence of the amino acid sequence in i) said subsequence comprising 1-38 amino acid residues, starting with the C-terminal K in the amino acid sequence in i)
and
xx₂ consists of
iii) The amino acid sequence DTMQIVSLQLNKMKSRKSCGIAVGTTIVDA (SEQ ID NO: 22) or
iv) A subsequence of the amino acid sequence in iii) said subsequence comprising 1-29 amino acid residues, starting with the N-terminal D in the amino acid sequence in iii).
   In these embodiments, the sequences may be chosen from SEQ ID NO: 23-28, 49-59.

Polypeptides according to any of the above embodiments are also provided additionally comprising a fragment comprising the variable domains 1 (VD1) of MOMP and wherein the amino acid sequences comprising VD1 of MOMP are placed next to each other or are spaced with a linker. In an embodiment thereof is a polypeptide comprising an amino acid sequence defined in formula II:

yy₁-VD1-yy₂ (Formula II)
wherein
VD1 is independently selected from SEQ ID NO: 1-6 or an amino acid sequence which has at least 80% sequence identity herewith and
yy₁ consists of
v) The amino acid sequence DAISMRVGYYGDFVFDRVLKTDVNKEFQMG SEQ ID NO: 7) or
vi) A subsequence of the amino acid sequence in v) said subsequence comprising 1-30 amino acid residues, starting with the C-terminal G in the amino acid sequence in v) and
yy₂ consists of
vii) The amino acid sequence NPAYGRHMQDAEMFTNAA (SEQ ID NO: 8) or
viii) A subsequence of the amino acid sequence in vii) said subsequence comprising 1-18 amino acid residues, starting with the N-terminal N in the amino acid sequence in vii).
   In these embodiments, the sequences may be chosen from SEQ ID NO: 9-14, 45-48.

Polypeptides according to any of the above embodiments are also provided comprising a fragment comprising the variable domains 2 (VD2) of MOMP and wherein the amino acid sequences comprising VD2 of MOMP are placed next to each other or are spaced with a linker. In an embodiment thereof is a polypeptide comprising an amino acid sequence defined in formula III:

zz₁-VD2-zz₂ (Formula III)
wherein
VD2 is independently selected from SEQ ID NO: 29-34 or an amino acid sequence which has at least 80% sequence identity herewith, and
zz₁ consists of
ix) The amino acid sequence TLGATSGYLKGNSASFNLVGLFG (SEQ ID NO: 35) or
x) A subsequence of the amino acid sequence in ix) said subsequence comprising 1-23 amino acid residues, starting with the C-terminal G in the amino acid sequence in ix)
and
xx₂ consists of
xi) The amino acid sequence VVELYTDTTFAWSVGARAALWE (SEQ ID NO: 36) or
xii) A subsequence of the amino acid sequence in xi) said subsequence comprising 1-22 amino acid residues, starting with the N-terminal V in the amino acid sequence in xi).

Polypeptides according to any of the above embodiments are also provided comprising a fragment comprising the variable domains 3 (VD3) of MOMP and wherein the amino acid sequences comprising VD3 of MOMP are placed next to each other or are spaced with a linker. In an embodiment thereof is a polypeptide comprising an amino acid sequence defined in formula IV:

qq₁-VD3-qq₂ (Formula IV)
wherein
VD3 is independently selected from SEQ ID NO: 37-42 or an amino acid sequence which has at least 80% sequence identity herewith, and
qq₁ consists of
xiii) The amino acid sequence ATLGASFQYAQSKPKVEELNVLCNAAEFTINKPKGYVG (SEQ ID NO: 43) or
xiv) A subsequence of the amino acid sequence in xiii) said subsequence comprising 1-22 amino acid residues, starting with the C-terminal G in the amino acid sequence in xiii)
and
qq₂ consists of
xv) The amino acid sequence TGTKDASIDYHEWQASLALSYRLNMFTPYIGVKWS (SEQ ID NO: 44) or
xvi) A subsequence of the amino acid sequence in xv) said subsequence comprising 1-35 amino acid residues, starting with the N-terminal T in the amino acid sequence in xv).

Polypeptides according to any of the above embodiments are also provided comprising a moiety that facilitate export of the polypeptide when produced recombinantly (e.g. signal peptides), a moiety that facilitate purification of the fusion protein (e.g. his-tags) and/or a moiety which enhance the immunogenicity (e.g. a T cell antigen). In some embodiments, the enhancer of immunogenicity is an additional T-cell target which is chosen from a Ct antigen such as CT043, CT004, CT414, CT681 or part hereof. In these embodiments, said sequences may be chosen from SEQ ID NO: 60-68.

Still further provided are polypeptides according to any of the above embodiments, said polypeptide having the ability to
a) neutralize C. trachomatis serovar D in vitro with a 50% neutralization titer of 10⁻³ or less, when tested in an experimental set-up comprising the administering heterologous immuno-repeats
b) neutralize C. trachomatis serovar D in vivo in at least 50% of the mice at day 7 post infection when tested in a mouse model comprising administering heterologous immuno-repeats
c) broaden the immune response to multiple serovars of C. trachomatis in vitro when administering a heterologous of immuno-repeats

Still further provided are nucleic acids encoding a polypeptides according to any of the above embodiments.

Also provided are pharmaceutical compositions comprising a polypeptide according to any of the above embodiments or a nucleic acid according to any of the above embodiments. The pharmaceutical compositions may be vaccines. The pharmaceutical compositions may additionally comprise a pharmacologically acceptable carrier, excipient, adjuvant or immune modulator. The pharmaceutical compositions may include an adjuvant selected from DDA/TDB or alum. In further embodiments, pharmaceutical compositions may include a carrier that is a virus-like particle.

Still further provided are pharmaceutical compositions comprising a polypeptide according to any of the above embodiments or a nucleic acid according to any of the above embodiments for prophylactic or therapeutic use against Chlamydia sp. infections, including infections with Chlamydia trachomatis or C. pneumoniae.

Methods for preventing, treating and/or reducing the incidence of Chlamydia sp. infections, including infections with Chlamydia trachomatis and C. pneumoniae, said method comprising administering a pharmaceutical composition described herein are also provided.

### Material and Methods

Cultivation of C. trachomatis

Ct serovar D, E and F was propagated in Hela 229 cells (ATCC, Rockville, MD, USA). The cells were cultivated in RP MI 1640 (Gibco BRL, Grand Island, NY, USA) media containing 5% fetal calf serum (Gibco BRL; heat inactivated), 1% v/v Hepes, 1% v/v L-glutamine, 1% v/v pyrovate and 10 μg/ml gentamycine. Semiconfluent monolayers of Hela 229 cells in 6 well-plates were infected with 1.5 inclusion forming unit per cell of Ct serovar E or F in 0.3 ml SPG-buffer/well. The plates were centrifuged 1 hour in a Heraeus Multifuge 3S at 750 g and incubated on a plate rocker for 2 h at 35° C. After 2 h 2 ml cultivation media supplemented with 5% glucose and 1 μg/ml cycloheximid were added pr. well and the cells were further incubated for 72 h at 37° C. in an atmosphere of 5% CO₂ in humidified air.

### Harvesting of Ct

Chlamydiae were harvested 72 h post infection. The cells were dislodged from the wells with a cell scraper and centrifuged 30 minutes at 35.000 g and 4° C. The pellets were resuspended in HBSS, sonicated on ice and centrifuged at 500 g and 4° C. for 15 minutes. The supernatant was collected and saved on ice and the pellet was resuspended to same volume as before and sonication and centrifugation were repeated. The two supernatants were pooled and centrifuged 30 minutes at 30000 g and 4° C. and the pellet resuspended with a needle and syringe in a SPG buffer (3 ml/Plate). After a brief sonication the suspension was gently layered over a 30% Diatrizoate solution (50 g Meglumine diatrizoate, 7.7 g Sodium diatrizoate in 76 ml H₂O) and centrifuged at 40,000 g for 30 min. After centrifugation the pellet were resuspended in SP G buffer and stored at −70° C. The IFU of the batches were quantified by titration on McCoy cells and the concentration of the batches was determined by BCA.

### Antigen and Fusion Preparation Methods

The genome of C. trachomatis serovar D, E, F and G are publicly available (NCBI-GenBank). Genes coding for C. trachomatis antigens and fusions where all obtained synthetically for cloning into E. coli bacterial protein expression system (DNA2.0). The pET411 vector was used for expression of the recombinant C. trachomatis protein in E. coli with a Histidine affinity tag. The bacterial host was BL21-STAR™. E. coli was grown at 37° C. to reach the logarithmic phase OD600 ˜0.5 and protein expression was induced for 4 hours and cells were harvested by centrifugation (6,000 g for 15 min.). E. coli were lysed using Bugbuster (Novagen) containing Benzonase, rLysozyme and Protease inhibitor Cocktail I (C albiochem). Inclusion bodies were isolated by centrifugation (10,000 g for 10 min.) The pellet was dissolved in 50 mM NaH2PO4, 0.4M NaCl, 8M Urea, 10 mM Imidazole pH 7.5 and loaded onto HisTrap HP column (Amersham Biosciences) and bound proteins were eluted by applying a gradient of 50 to 500 mM imidazole. Depending on the antigen and fusions isoelectric point they were further purified by ion exchange chromatography. Protein concentrations was determined by BCA protein assay (Pierce).

### Animals

Female B6C3F1 mice, 8-12 weeks of age, were obtained from Harlan Laboratories. Animals were housed under standard environmental conditions and provided standard food and water ad libitum. The use of mice is guided by the regulations set forward by the Danish Ministry of Justice (Lov om dyreforsøg, jvf lovbekendelser nr. 726 af 9. September 1993), and Animal protection committees. A detailed description of the experiments was submitted to and approved by the regional ethical review board (2012-15-2934-00100) held by the applicant

### Immunization

Mice were immunized 3 times with 14 days between immunizations. The poly peptides were emulsified in CAF01 and administered simultaneously by the subcutanous (sc) and intranasal (i.n) route. The vaccines given by both routes consisted of 5 ug of peptide (see above) emulsified in 250 ug DDA and 100 ug TDB. As a negative control, DDA/TDB alone, without peptide was injected.

### Chlamydia-Specific Cellular Responses

Blood lymfocytes or splenocytes were purified. Blood lymphocytes were pooled from 8 mice in each group and spenocytes were cultivated individually (n=4) and cultured in triplicate in round-bottomed microtiter plates (Nunc, Denmark) containing 2×10⁵ cells/well in a volume of 200 μl RPMI-1640 supplemented with 5×10⁻⁵ M 2-mercaptoethanol, 1 mM glutamine, 1% pyruvate, 1% penicillin-streptomycin, 1% HEPES and 10% fetal calf serum (FCS) (Invitrogen, Denmark). The cells were re-stimulated with individual antigens in 1-10 μg/ml or VD1 and VD4 peptide pools (2 μg/ml of each peptide). Stimulation with Concanavalin A (5 μg/ml) or media as positive control for cell viability and negative control, respectively. After 72 h of incubation at 37° C. in 5% CO₂, supernatants were harvested and stored at −20° C. before use. The amounts of secreted IF N-γ were determined by enzyme-linked immunosorbant assay (ELISA).

### Serum Antibodies

At different time points post last vaccination the mice were bled and serum isolated by centrifugation. Serum was tested by ELISA for reactivity against the Ct surface (S vD, S vE and S vF), against the S vE VD4 monomer, and against peptides (Table 4&5) spanning the VD4 region of S vD, S vE and S vF. Briefly, plates were coated with antigen (1 to 10 ug/ml) at 4° C. in carbonate buffer overnight, blocked with BSA and washed. The plates were then incubated with pre-diluted samples at 4° C. overnight, washed and incubated with a peroxidase conjugated secondary antibody for 1 hr. Reactions were visualized by incubation with TMB substrate and the reaction stopped with sulphuric acid and read at 450 nm. When ELISA reactivity against a 9mer overlapping peptide panel spanning the VD4 region of S vD (S vE) (Table 6) and S vF (Table 7) was investigated minor changes were done. Briefly, plates were treated with streptavidin and coated with biotinylated peptides, blocked for 2 h at room temperature with skimmed-milk powder and washed. The plates were then incubated with pre-diluted (1:100) serum samples for 2 h at room temperature, washed and incubated with a peroxidase conjugated secondary antibody for 1 hr. Reactions were visualized by incubation with TMB substrate and the reaction stopped with sulphuric acid and read at 450 nm.

### Neutralization Assay

Hak cells were grown to confluence in 96-well flat-bottom microtiter plates in RP MI 1640 media supplemented with 5% fetal calf serum (Gibco BRL; heat inactivated), 1% v/v Hepes, 1% v/v L-glutamine, 1% v/v pyrovate and 10 μg/ml gentamycine.

The Chlamydia stocks were previously titrated and diluted to 3×10⁶ IFU/ml for S vE, 2×10⁶ IF U/ml for S vD and 5×10⁶ IFU/ml for S vF. Serum (pooled) isolated from vaccinated mice was heat inactivated at 56° C. for ½ h, diluted 2-4 times and 4-5 fold titrated. 80 μl of the bacteria suspension was mixed with 80 μl of serum (+/−20 μg/ml peptide) and incubated for 30 min. at 37° C. on a slowly rocking platform and 50 μl of the suspension were then inoculated onto the previously prepared Hak cells in dublicates. To do this, the media was removed from the Hak monolayers and 100 μl of the above media supplemented with 0.5% glucose and 10 μg/ml cyclohexamide was added followed by 50 μl of the serum/bacteria suspension. Plates were incubated at 35° C. on a slowly rocking platform, then inoculum was removed and 100 μl of the above media supplemented with 0.5% glucose and 10 μg/ml cycloheximide was added. The plates were then incubated for 24 h at 37° C. in an atmosphere of 5% CO₂ in humidified air. After incubation the medium was removed and the monolayers were fixed with 96% ethanol for 10 min. Inclusions were visualized by staining with polyclonal rabbit anti-CT755 serum made in our laboratory, followed by FITC-conjugated swine anti-rabbit immunoglobulin (Dako). Background staining was done with propidium iodide (Invitrogen)

### Vaginal Challenge and Vaginal Chlamydial Load

Ten and 3 days before Ct serovar D challenge, the oestrus cycle was synchronized by injection of 2.5 mg Medroxyprogesteronacetat (Depo-Provera; Pfizer). Six weeks after the final vaccination the mice were challenged i.vag. with 4-8×10⁵ IFU of Ct serovar D in 10 μl SPG buffer. Vaginal swabs were obtained at 3, 7, 10 and 14 days after infection. Swabs were vortexed with glass-beads in 0.6 ml SPG buffer and stored at −80 C until analysis. Infectious load was determined as described in 17. Briefly, McCoy cell monolayers were infected with a titrated volume of the swab suspension in duplicates. The plates were centrifuged at 750×g for 1 h at RT followed by incubation at 35 C for 2 h. Infection-media was then replaced with fresh media and the cells incubated at 37 C for 30 h. Inclusions were visualised by staining with polyclonal rabbit anti-CT681 serum made in our laboratory, followed by a FITC conjugated swine anti-rabbit Ig (DAKO, Glostrup, Denmark). Background staining was done with propidium iodide (Invitrogen, Taastrup, Denmark). Inclusions were enumerated by fluorescence microscopy observing at least 20 individual fields of vision for each well.

Depletion of CD4⁺ and CD8⁺ T-Cells

Monoclonal anti-mouse CD4 (clone GK1.5) and anti-mouse CD8 (clone YTS 156 and YTS 169 a gift from Stephen Cobbold) ^{78, 79} was purified from hybridoma supernatants made in our lab, using HiTrap protein G HP columns (GE-Healthcare Life Sciences, Denmark). The purified IgG was dialyzed against PBS, filtered through 0.22 μm filter and protein concentration was determined by OD 280 nm. Mice were depleted of CD4⁺ or CD8⁺ T-cells by 4 injections of 250-300 μg purified anti-CD4 or a mix of anti-CD8 antibodies at day −7, −4, −1 and +2 and +6 relative to the day of infection. The CD4⁺ and CD8⁺ T cell depletions were verified by FACS analysis on PBMCs at day 1 post infection using a FITC conjugated anti-CD4 antibody (clone RM4-4) and a PE-conjugated anti-CD8 antibody (clone 53-6) (BD Biosciences, Denmark).

### In Vivo Depletion

The Chlamydia serovar D stock was previously titrated and diluted to 8×10⁴ IFU/μl, mixed 1:1 with serum isolated from mice immunized with a heterologous VD4 immuno-repeat S vD-S vE-S vF (CTH89). Ten and 3 days before Ct serovar D challenge, the oestrus cycle was synchronized by injection of 2.5 mg Medroxyprogesteronacetat (Depo-Provera; Pfizer). Mice were challenged i.vag. with 10 μl of the above mix (4×10⁵ IFU of Ct serovar D). Vaginal swabs were obtained at 3, 7 and 10 days after infection.

### Statistical Analysis

Statistical analysis was done using GraphPad Prism 4. Medians of vaginal Chlamydia load were analyzed using Kruskall-Wallis followed by Dunn's post test or Mann-Whitney.

### Example 1: Enhanced Immune Responses after Immunization with Homologous Immuno-Repeats of VD4^{ext} Compared with a Monomeric VD4^{ext} Unit

### Introduction

Here we selected polypeptide units containing extended VD4 fragments of serovar E (for sequence see FIG. 2) (S vE VD4^{ext}). In order to potentiate the immune response against these domains we designed recombinant polypeptides were the SvEVD4^{ext} unit was presented in a repetitive manner. To investigate if a repetitive form of the construct could enhance the antibody response compared to a monomeric form, we designed recombinant polypeptides where the units were presented either as a single unit or in a repetitive manner. For serovar E (S vE), a monomeric (S vE VD4^{ext})*1 (CTH181), four immuno-repeats (S vE VD4^{ext})*4 (CTH527) and eight immuno-repeats (S vE VD4^{ext})*8 (CTH526) of the extended VD4 unit were constructed. These homologous immuno-repeat constructs were formulated in the adjuvant CAF01 and used to vaccinate mice; each mice was vaccinated with 2×5 μg peptide so the amount of VD4 was the same. Immunogenicity of the constructs was studied by ELISA against S vE VD4^{ext}, peptides covering S vE VD4^{ext} and the bacterial surface of chlamydia.

### Results

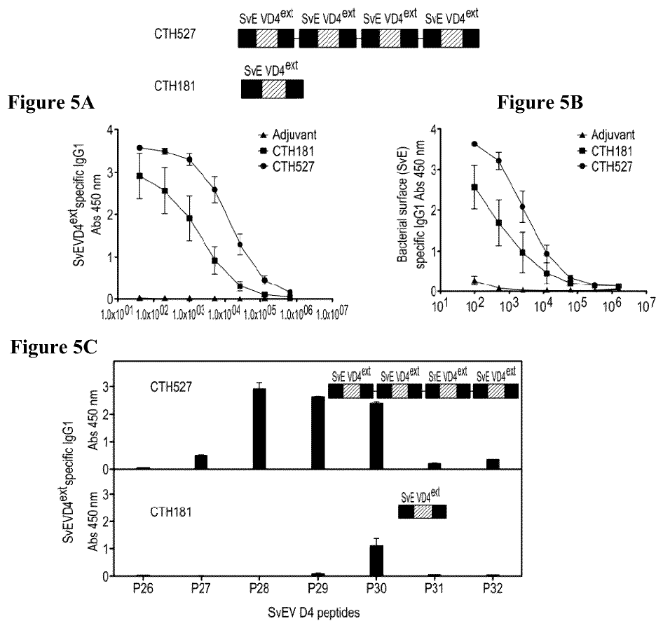

Six mice/group were immunized 2 times with 14 days between immunizations. The vaccines (2×5 μg) were emulsified in CAF01 and administered simultaneously by the sc. and i.n routes. At certain time points post last vaccination blood was collected and antibody levels against the extended VD4 units from S vE and against the bacterial surface of S vE were measured by ELISA. Vaccination with a single VD4^{ext} unit (monomeric VD4^{ext}, CTH181) induced lower levels of VD4^{ext} specific antibodies compared to the level induced after immunization with homologous immuno-repeats composed of 4 VD4^{ext} repeats of (S vE VD4^{ext})*4 (FIG. 5A). The higher antibody response seen after immunizing with (S vE VD4^{ext})*4 resulted in a stronger recognition of the bacterial surface compared to serum isolated from (VD4^{ext})*1 immunized mice (FIG. 5B). The response to 20mer peptides with 10aa overlap spanning the extended VD4 region (Table 4) was also enhanced resulting in a broader epitope recognition pattern in the (VD4^{ext})*4 homologous immuno-repeat groups compared to the group of mice immunized with a monomeric VD4^{ext} unit when tested in a 1:500 serum dilution (FIG. 5C). In the group immunized with the monomeric construct the response was exclusively targeted to the central region containing the TTLNPTIAG (SEQ ID NO: 76) epitope whereas immunization with the homologous immuno-repeat exposed several B cell epitopes both up- and downstream of that epitope resulting in a diverse epitope recognition pattern of various epitopes. We continued by investigating if immuno-repeats of 8 (S vE VD4^{ext})*8 (CTH526, seq no 30) were more immunogenic than immuno-repeats of 4 (S vE VD4^{ext})*4. The two constructs induced similar levels of antibodies against the extended VD4 unit and against the bacterial surface of S vE.

### Conclusion

We demonstrated that by immunizing with immuno-repeats of extended VD4 units from Serovar E we can greatly enhance antibody response both measured as the titer (FIGS. 5A&B) and the breadth of the response (FIG. 5C) directed against the extended VD4 unit resulting in a strong reactivity towards the bacterial surface. We did not find enhanced antibody titers and neutralization titers by increasing the number of repeats from 4 to 8.

### Example 2: A Construct Composed of Heterologous Immuno-Repeats from S vD, E, F and G (CTH518) Induced a Stronger Response to Multiple Serovars Compared to Homologous Immuno-Repeats from S vF

### Introduction

We investigated if immunization with at heterologous immuno-repeat composed of extended VD4 units from S vD, S vE, S vF and S vG (CTH518), maintained the strong immunogenicity and was able to induce a broader antibody response recognizing the surface of multiple serovars compared to immunization with a homologous immuno-repeat composed of extended VD4 units from S vF (S vF VD4^{ext})*4, (CTH529). These immuno-repeat constructs were formulated in the adjuvant CAF01 and used to vaccinate mice. The immunogenicity of the constructs was studied by ELISA against the bacterial surface of Serovar D, E and F.

### Results

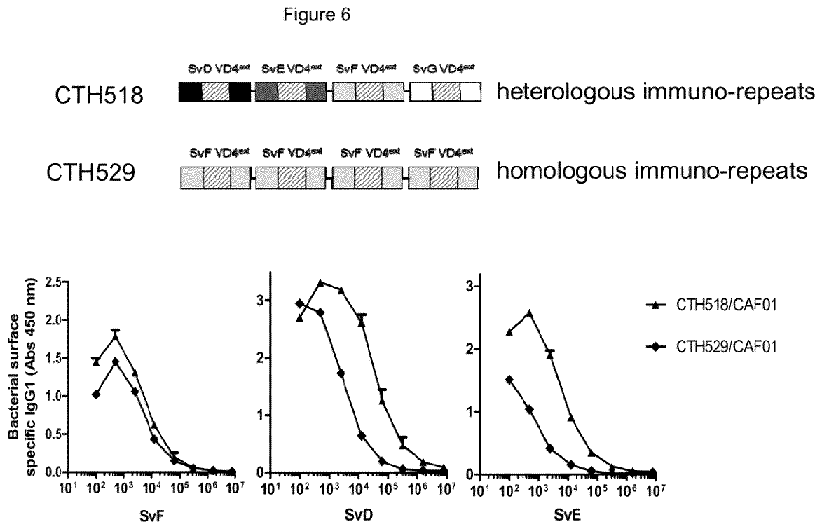

Heterologous immuno-repeats promoted an antibody response that recognized the surface of the serovar F strain at the same high level as the response seen with a homologous immuno-repeat from S vF. However, by immunization with the heterologous immuno-repeat containing extended VD4 regions from the four serotypes (S vD, S vE, S vF, S vG) we observed a markedly increased titer to the D and E serovariants compared to the homologous immuno-repeat from the serovar F (FIG. 6).

### Conclusion

Immunizing with the construct composed of immuno-repeats of heterologous extended VD4's induced a broader response recognizing the surface of multiple serovars (D, E and F) while maintaining the pronounced immunogenicity of the homologous immuno-repeat.

### Example 3: The Specificity of the Antibody Responses after Immunization with a Heterologous Immuno-Repeat of the Extended VD4 Units from Serovar D, E, and F (CTH89) Compared to Constructs Composed of a Homologous Immuno-Repeat from (S vE^{ext} VD4)*4, (S vF^{ext.} VD4)*4 and a Previously Published A8-VD4 Peptide ⁶⁵

### Introduction

We investigated the specificity of the immune response after immunization with a heterologous repeat of extended VD4 domains from S vD, S vE, S vF (CTH89) compared to immunization with homologous immuno-repeats composed of extended VD4 repeats from Serovar E (S vE^{ext}VD4)*4 (CTH527), S vF (S vF^{ext}VD4)*4 repeats (CTH524) and A8-VD4 peptide. These constructs were formulated in the adjuvant CAF01 and used to vaccinate mice. Immunogenicity of the constructs was studied by ELISA against a peptide panel (9 and 20 AA long) spanning the VD4 region of D, E and F (Tables 4-7). Serum (from 6 to 8 mice) was tested and a response above background but below OD=1.0 is indicated by an open box, responses above 1.0 are marked by a filled box. The length of the box indicates the area recognized by antibodies.

### Results

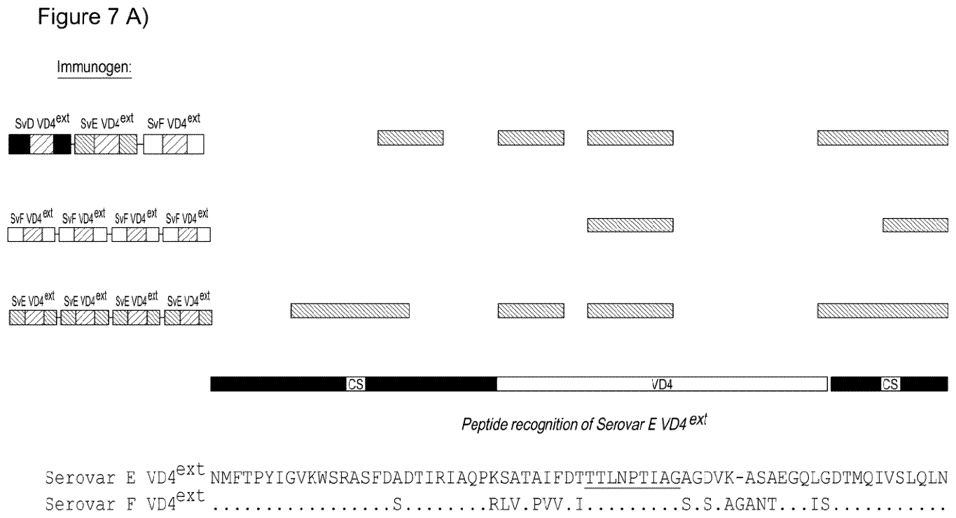

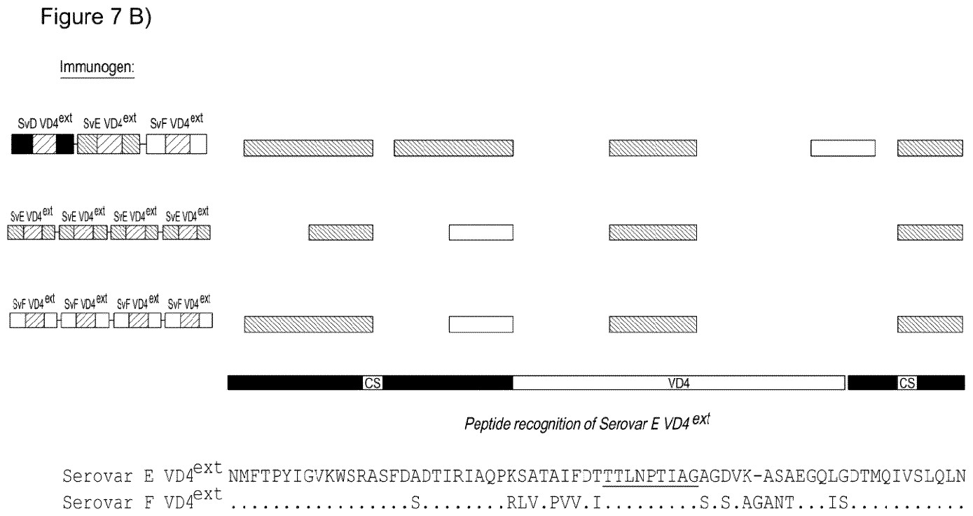

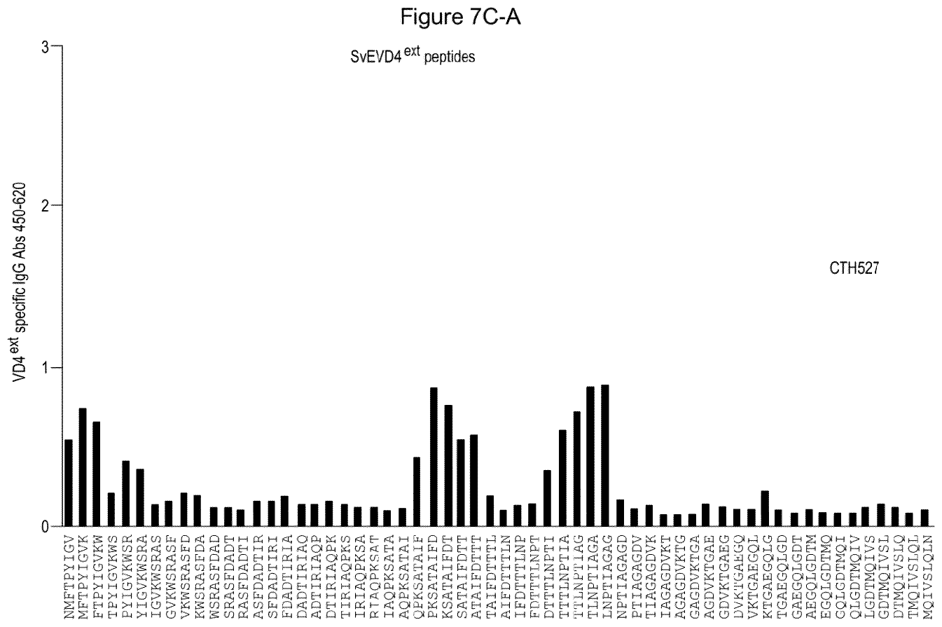

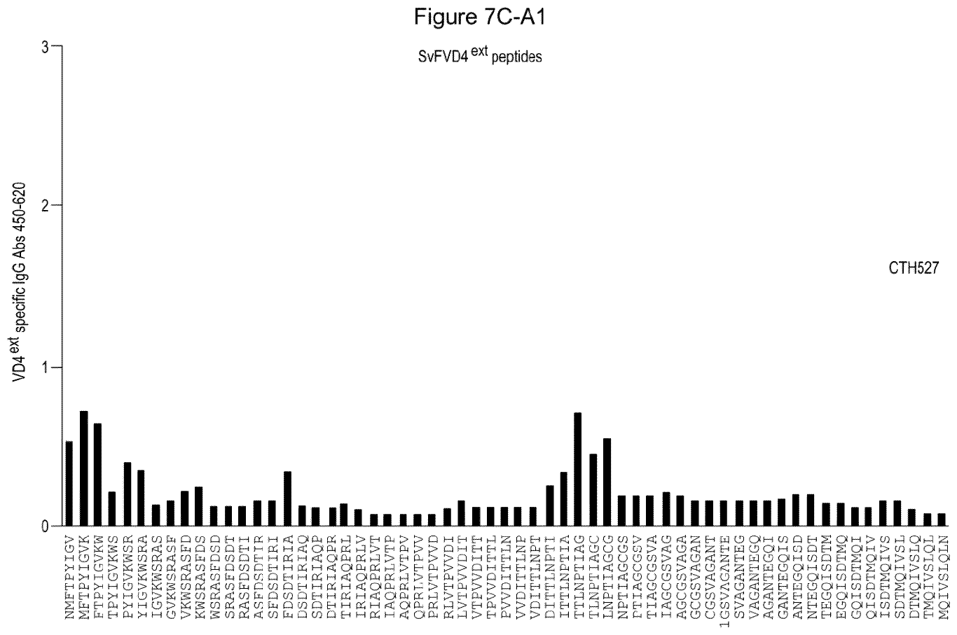

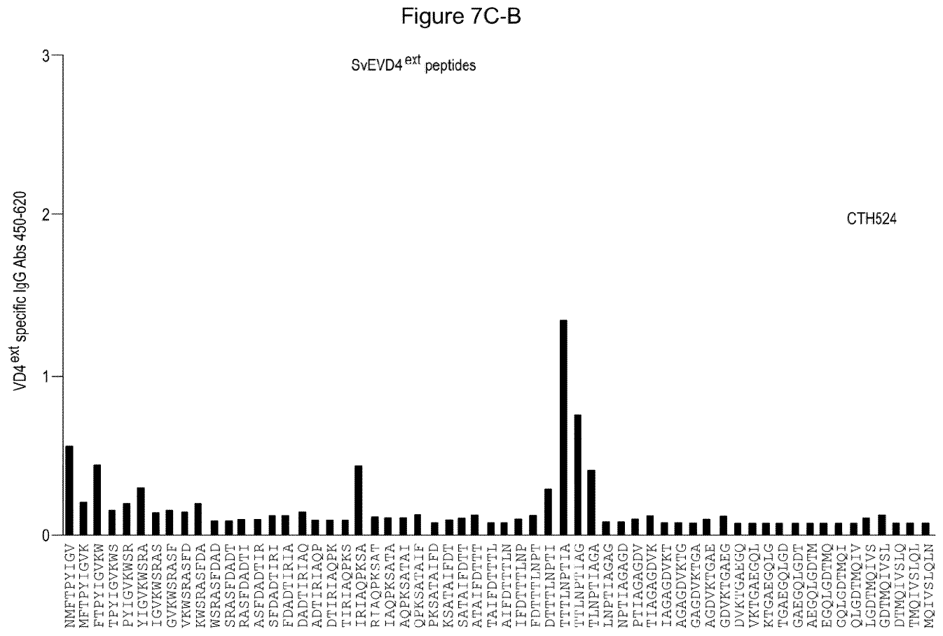

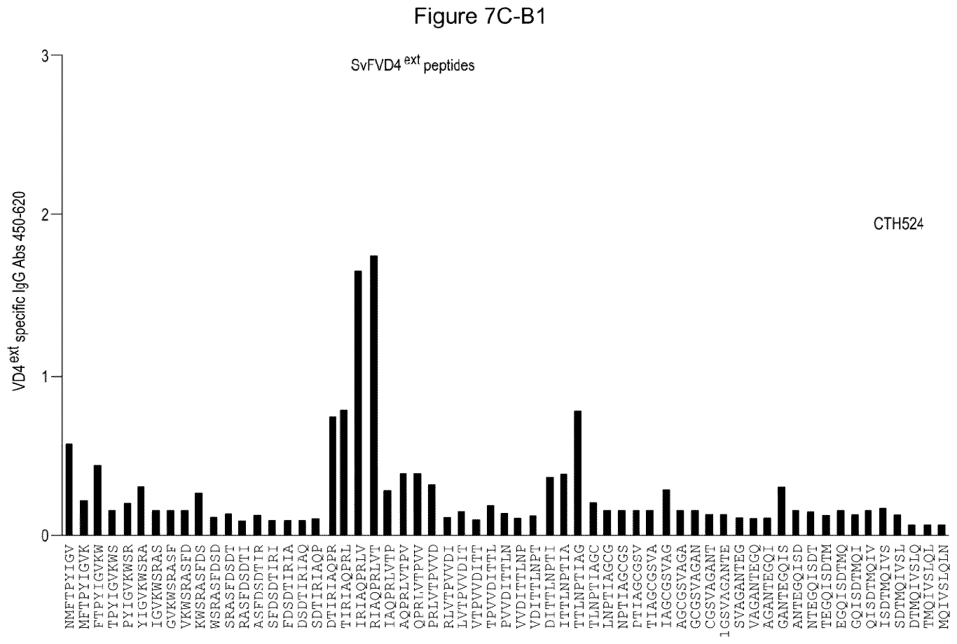

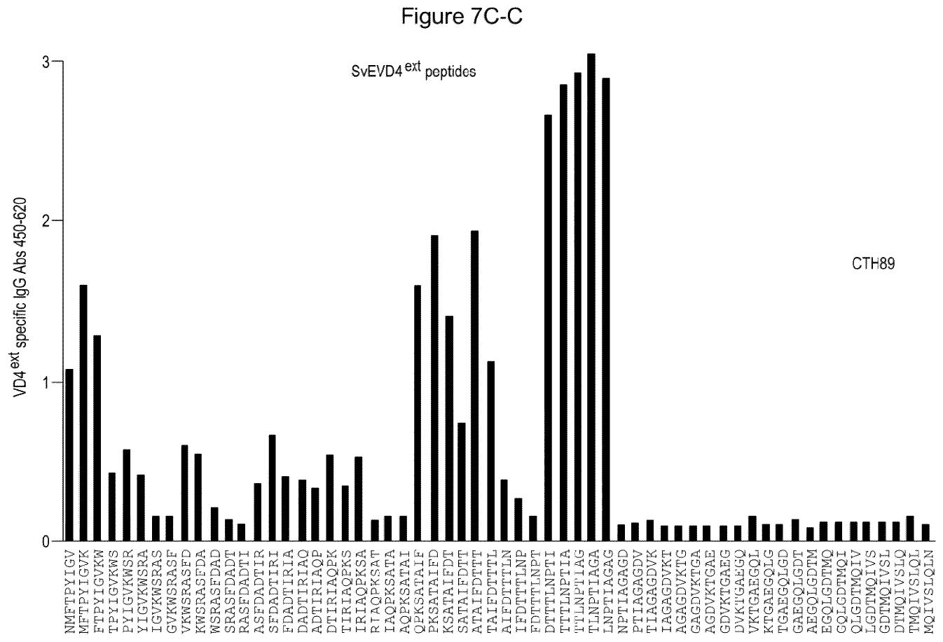

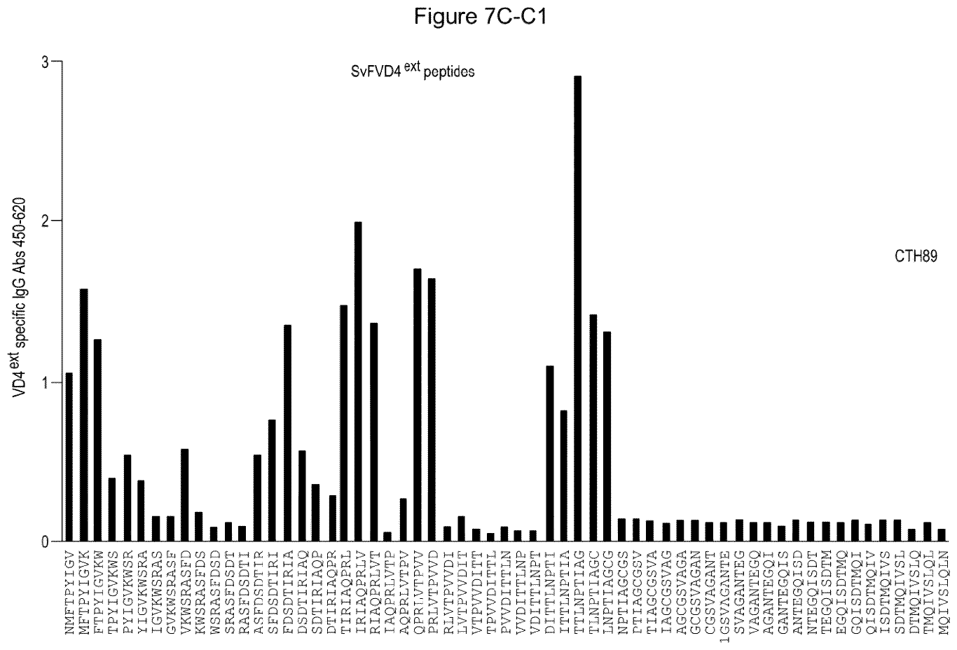

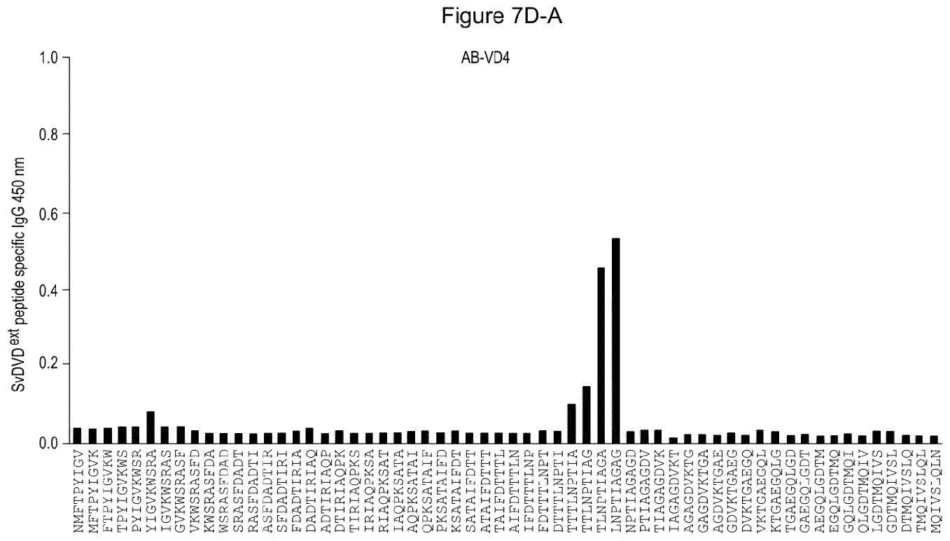

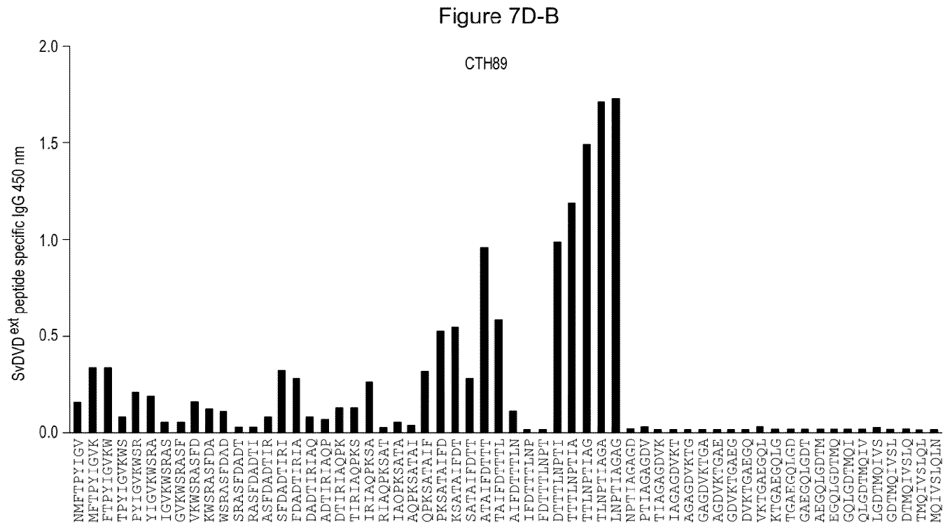

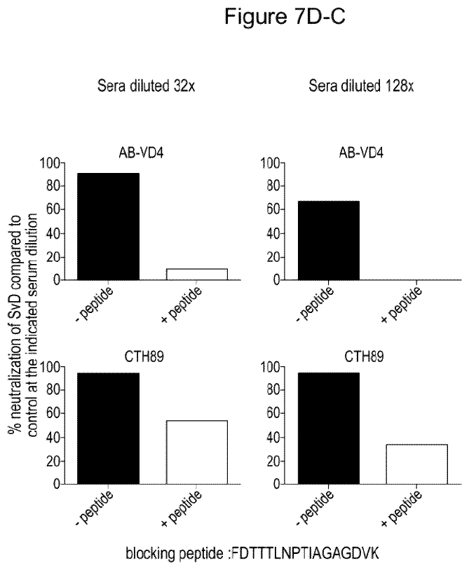

All constructs induced high antibody responses to the conserved TTLNPTIAG (SEQ ID NO: 76) part of the VD4^{ext}, located in the variable domain (VD). In general antibodies generated by homologous immuno-repeats were superior in recognizing their representative homologous VD4^{ext} region, whereas it was evident that when these constructs were tested against peptides covering a VD4^{ext} from a different serovar their epitope recognition repertoire was limited e.g. the recognition of serovar E VD4 region by serum from animals immunized with the construct (S vF^{ext}VD4)*4 (FIG. 7A and FIGS. 7C-A, 7C-B, and 7C-C) (and vice versa) (FIG. 7B and FIGS. 7C-A1, 7C-B1, and 7C-C1). Antibodies generated after immunization with the heterologous immuno-repeats (CTH89), recognized a much broader epitope repertoire than serum from animals immunized with the homologous immuno-repeats and the A8-VD4 (FIGS. 7A-7D-B). This construct was able to cover an epitope repertoire covering both serovar E and F at the level (or better) than achieved by immunizing with homologous immuno-repeats.

To demonstrate whether a 17 AA peptide representing a central VD4 peptide FDTTTLNPTIAGAGDVK (SEQ ID NO: 194) was able to compete with C. trachomatis organisms for CTH89 specific antibody binding, a competitive neutralization assay was performed. Different concentrations of CTH89 and A8-VD4 specific serum were mixed with the peptide in a concentration of 20 μg/ml (FIG. 7D-C). The results demonstrates that, in contrast to A8-VD4 specific serum, the peptide could not completely eliminate the neutralizing capacity of the CTH89 specific serum, suggesting that this serum targets a broader repertoire of neutralizing epitopes.

### Conclusion

Immunizing with immuno-repeats of heterologous extended VD4's induced a broad response recognizing both conserved and serovar specific parts of the VD4 region, translating into a broader repertoire of neutralizing epitopes.

### Example 4: Immunization with Heterologous Immuno-Repeats of Extended VD4's from S vD, S vE and S vF (CTH89) Generates Early T Cell Independent Protection after a S vD Challenge

### Introduction

In order to study the effector mechanism responsible for the early protection seen after vaccination with the VD4 repetitive units, mice vaccinated with CTH89 were T cell depleted before challenge and the capacity to induce early protection was compared in depleted and non-depleted mice.

### Results

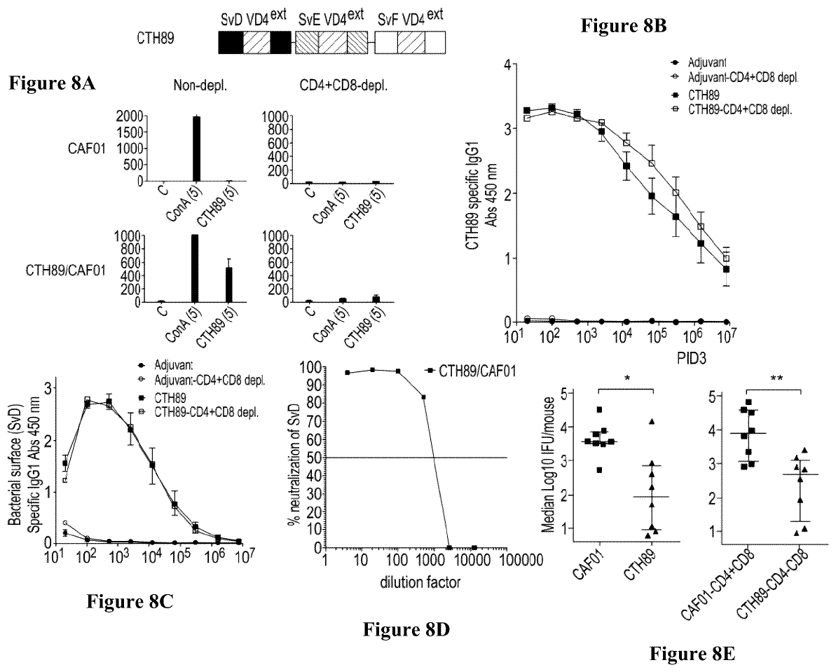

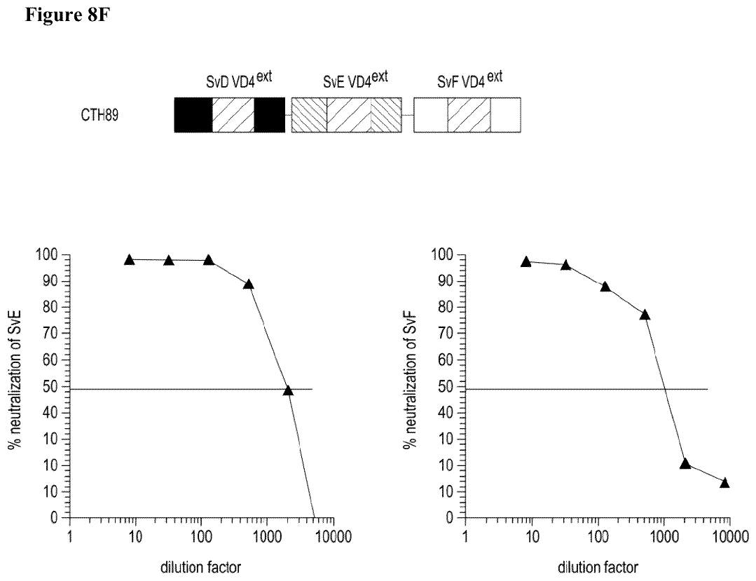

Eight mice/group were immunized 3 times with 14 days between immunizations. The vaccine (2×5 μg) was emulsified in CAF01 and administered simultaneously by the sc. and i.n routes. At certain time points post last vaccination the mice were bleed and antibody responses against chlamydia, the neutralization titer, and in vivo protection with and without T cell depletion were measured. Depletion of the T cell subset eliminated the T cell response to CTH89 (FIG. 8A). CTH89 induced a strong antibody response (FIG. 8B) that recognized the surface of serovar D (FIG. 8C) and was able to neutralize the bacteria in vitro with a 50% neutralization titer of around 1:10³ (FIG. 8D). However, we still found significant protection at day 3 post challenge in the T cell depleted mice (FIG. 8E) suggesting an in vivo role for antibodies recognizing the VD4 unit in early protection against Chlamydia. Finally we demonstrated that CTH89 serum was also able to neutralize a S vE and S vF infection with very high 50% neutralization titers at the level of that obtained with S vD (FIG. 8F).

### Conclusions

Immuno-repeat generates T cell independent early protection against vaginal challenge with Serovar D suggesting an in vivo role of VD4 specific antibodies.

### Example 5: In Vivo Neutralization with CTH89 Specific Serum

### Introduction

In order to investigate if the in vitro neutralization could be translated to a protective effect mediated by serum in vivo, we next investigated if S vD bacteria coated with antibodies generated after CTH89 immunization could neutralize/inhibit the infection in vivo compared to serum from naive mice.

### Results

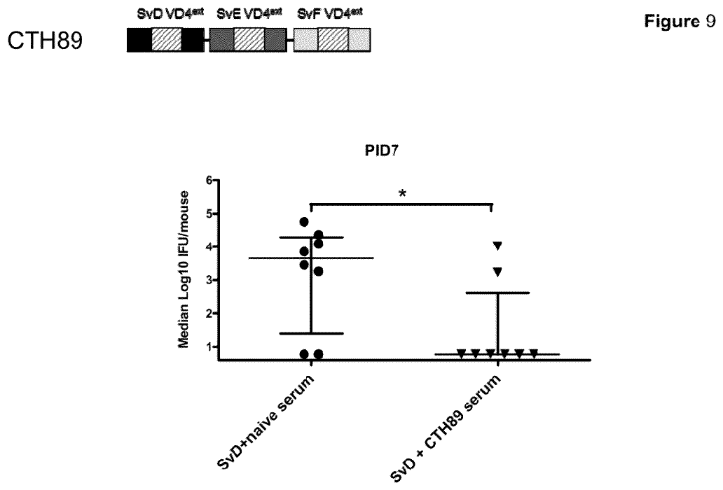

S vD bacteria were mixed with serum isolated from CTH89 immunized mice or serum isolated from naive mice. Depro-provera treated mice were then infected with 4×10⁵ bacteria. Mice infected with S vD coated with CTH89 serum efficiently controlled bacterial replication compared to mice challenged with S vD coated with naive serum. Six out of 8 mice were cleared at day 7 and 10 compared to 2 and 3 respectively, in the control group (FIG. 9).

### Conclusion

Serum generated after immunization with heterologous VD4 immuno-repeat efficiently block infection of mice with S vD compared to serum isolated from naive mice

### Example 6. Fusion of Recombinant MOMP with Immuno-Repeats of Heterologous Extended VD4's

### Introduction

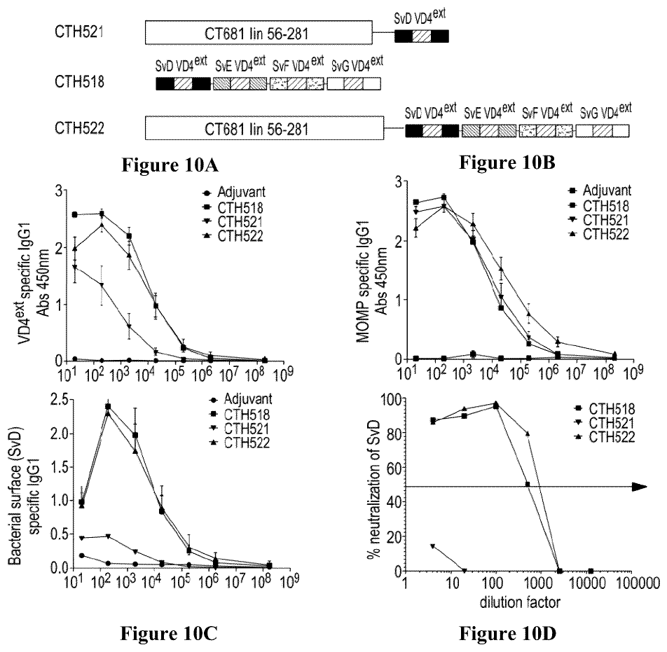

MOMP is the target of both humoral and cellular immune-responses but despite the relative success of refolded native MOMP vaccines in generating neutralizing antibodies and protect against infection ^{54, 56}, experimental vaccines based on recombinant MOMP (rMOMP) have failed. We designed a recombinant MOMP ranging from amino acid 56 to 349, including all variable domains (CTH521). We also selected polypeptide units containing extended VD4 fragments (covering the VD4 variable domain of MOMP and the adjacent conserved flanking regions) of serovar D, E, F and G (CT518) Finally a hybrid was constructed where CTH521 was fused to CTH518 (CT522) (FIG. 10).

### Results

Eight mice/group were immunized 3 times with 14 days between immunizations. The vaccines were emulsified in CAF01 and administered simultaneously by the sc. (5 μg) and i.n. (5 μg) routes. Post vaccination blood samples were collected and antibodies against the VD4^{ext} unit, recombinant MOMP and against the bacterial surface were measured. Antibodies generated after immunization with CT522 and CT518 recognized the VD4 region (FIG. 10A) and the bacterial surface (FIG. 10C) at a much higher level compared to serum isolated after CT521 immunization. Furthermore antibodies form CTH518 and CTH522 were able to neutralize a S vD infection at the same level and much higher than CTH521 (FIG. 10D).

### Conclusion

Fusion of recombinant MOMP with immuno-repeats of heterologous extended VD4's results in a molecule that elicits the same functional antibody response as the immune-repeat alone.

### Example 7: Vaccination with Heterologous Immuno-Repeats of VD1^{ext}-VD4^{ext}'s Regions from S vD, S vE and S vF (CTH88) Compared to Vaccination with a Single VD1-VD4 Unit from S vD (CTH87)

### Introduction

We next investigated if it was possible to fuse another VD region to the extended VD4 region and still maintain the capacity to induce neutralizing antibodies. Therefore constructs were designed were an extended version of the VD1 region was coupled to the extended VD4 region. We produced both a homologous unit composed of an extended unit of VD1 and VD4 from S vD (CTH87) and a heterologous immuno-repeat composed of extended units of VD1 and VD4 from different serovars (D, E and F; CTH88).

### Results

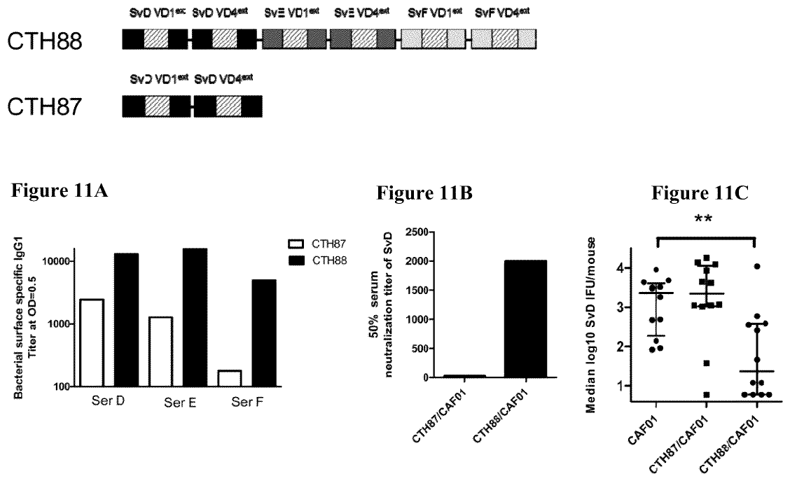

12 mice/group were immunized 3 times with 14 days between immunizations. The vaccines were emulsified in CAF01 and administered simultaneously by the sc. (5 μg) and i.n. (5 μg) routes Antibodies from mice immunized with CTH87 recognized the bacterial surface of both S vD, S vE and S vF (FIG. 11A); with the highest titers observed against the homologous S vD strain and the lowest titers against the most distant S vF. Immunizing with immuno-repeats of heterologous VD1^{ext}-VD4^{ext} units resulted in significant higher levels of antibodies against the surface of the bacteria compared to the monomeric construct and broadened the response resulting in titers increasing 6-12 times against S vD and S vE and almost 25 times against S vF (FIG. 11A). The capacity of these antibodies to neutralize infection in an in vitro neutralizing assay was even more improved as serum from animals immunized with the monomeric VD1^{ext}-VD4^{ext} construct from serovar D only had minimal neutralizing capacity compared to the heterologous VD1-VD4 immuno-repeat construct with a neutralization titer of 1:2000 (FIG. 11B). Finally did vaccination with the heterologous VD1^{ext}-VD4^{ext} immuno-repeat construct very efficiently protect against a S vD challenge in a vaginal challenge model (FIG. 11C).

### Conclusion

We demonstrated that by immunizing with immuno-repeats of heterologous VD1^{ext}-VD4^{ext} units from serovar D, E and F, we can greatly enhance the antibody response directed against the bacterial surface of all three serovariants. Importantly we also show that by vaccination with a heterologous immuno-repeat, we observe a selective higher increase in Serovar F surface recognition (25 times vs. 6-12 times for serovar D and E), suggesting that the heterologous immuno-repeats not only increase the antibody levels against shared epitopes but also against serovar F specific epitopes. We demonstrated that the antibodies induced with immuno-repeats of heterologous VD1-VD4 (CTH88) generated in vitro neutralizing titers that resulted in early in vivo protection compared to the single VD1-VD4 unit from S vD (CTH87) (FIG. 11C).

### Example 8: Coupling of T Cell Antigens to Immuno-Repeats of VD4

### Introduction

As there is a generally recognized need for a CMI component in an efficient protective immune response against Chlamydia trachomatis, we next investigated if the heterologous immuno-repeats can be fused to T cell antigens with vaccine potential. Our aim was to provide both an early antibody mediated protection against Ct as well as an efficient CMI mediated clearance of residual organisms. A constructs composed of CT043, and part of CT414 and CT681 was fused to immuno-repeats of heterologous VD1-VD4 (CTH91).

### Results

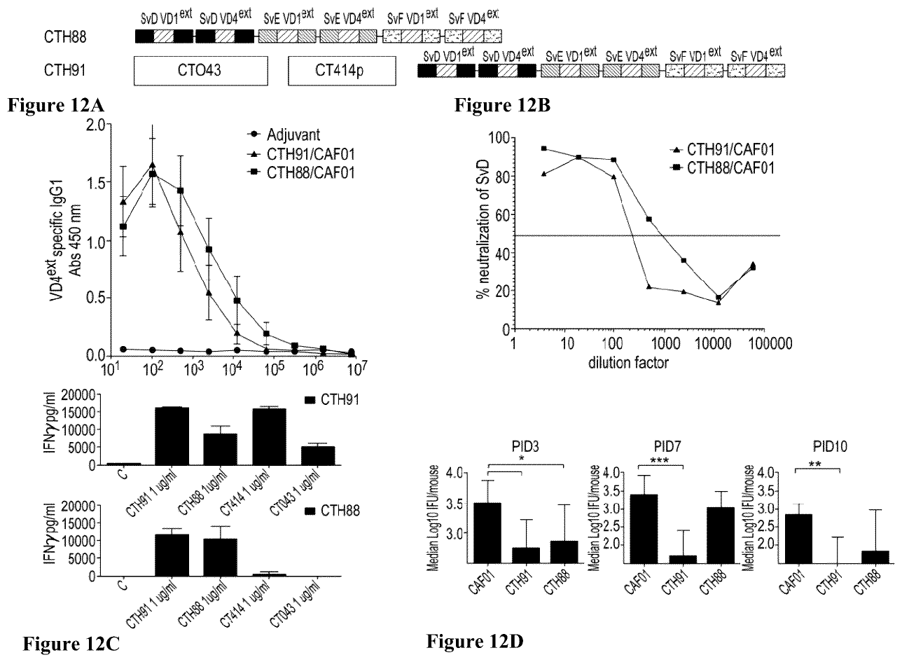

12 mice/group were immunized 3 times with 14 days between immunizations. The vaccines (2×5 μg) were emulsified in CAF01 and administered by the sc. and i.n. routes. At various time points post last vaccination the mice were bleed and antibody responses and neutralization titers were measured. Antibodies generated after immunization with CTH91 and CTH88 recognized the VD4^{ext} region at similar levels (FIG. 12A) and serum isolated from both groups were able to neutralize a S vD infection (FIG. 12B). Compared to CTH88 immunized mice the T cell response to CTH91 was stronger with recognition of both CT414 and CT043 (FIG. 12C). This T and B cell response resulted in significant protection at day 3 post infection for both groups, but at day 7 and 10 post infection the group vaccinated with a fused T and B cell target (CTH91) induced higher levels of protection compared to CTH88 (FIG. 12D).

### Conclusion

We were able to fuse T cell antigens with the repetitive VD regions and still maintain the capacity to induce early protection and moreover these constructs induced an efficient CMI mediated clearance of residual organisms leading to high levels of protection at day 7 post infection.

### Example 9: Immunization with a Cocktail of a Heterologous VD4 Immuno-Repeat and a T Cell Antigen Fusion Molecule

### Introduction

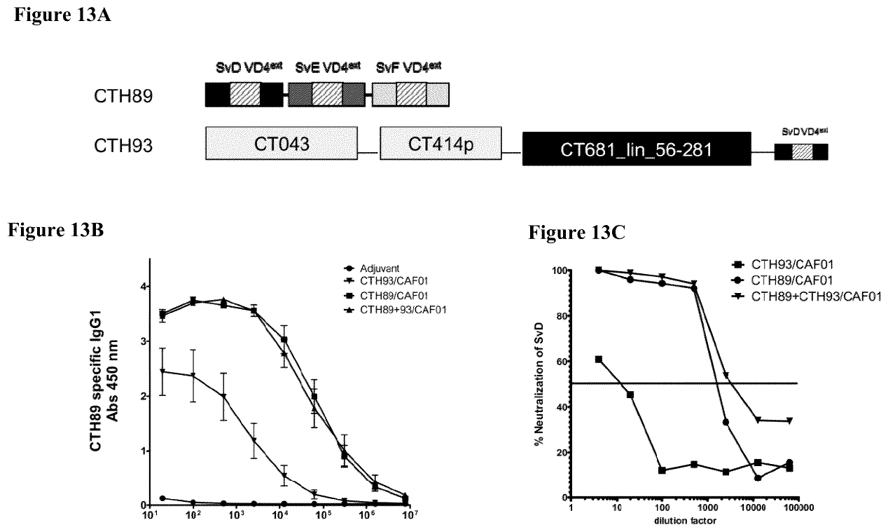

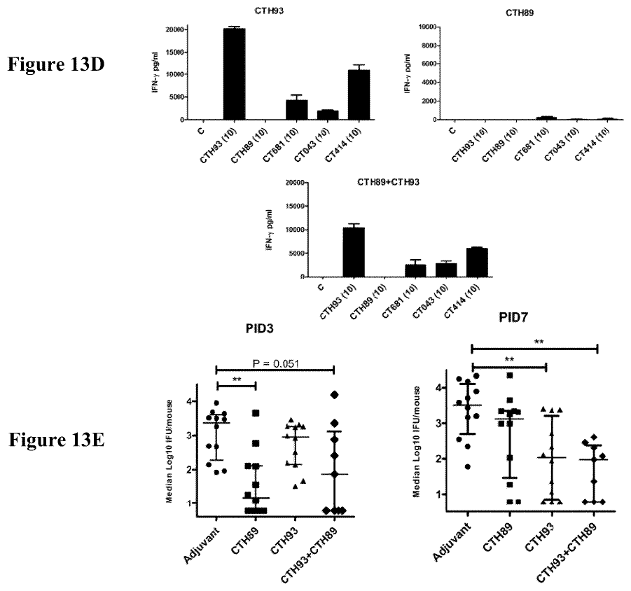

We next investigated if immuno-repeats can be mixed with T cell antigens with vaccine potential and still provide both an early antibody mediated protection against Ct as well as an efficient CMI mediated clearance of residual organisms. We therefore investigated if we could mix a strong T cell hybrid composed of CT043, part of CT414 and CT681 (CTH93) with CTH89 (FIG. 13A) and still maintain the capacity to neutralize the S vD bacteria in vitro and induce early protection against a vaginal challenge.

### Results

12 mice/group were immunized 3 times with 14 days between immunizations. The vaccine (2×5 μg) were emulsified in CAF01 and administered simultaneously by the subcutaneous (sc) and intranasal (i.n) route (FIG. 13). Antibodies generated after immunization with CTH89 or the mixture of CTH89 and CTH93 strongly recognized the VD4 regions (FIG. 13B) and neutralized the bacteria with similar 50% neutralization titers (FIG. 13C). Much reduced levels of VD4 recognition and neutralization was seen after vaccination with the T cell antigen fusion (CTH93, FIG. 13D) although this molecules also contained MOMP (CT681) and therefore potentially the same neutralizing epitopes. This molecule also gave very low levels of recognition of the TTLNPTIAG (SEQ ID NO: 76) epitope (data not shown). This clearly emphasizes the limitation of full-size recombinant MOMP as a vaccine antigen for the induction of neutralizing antibodies as previously reported. Both the CTH89 and the cocktail of the CTH89 and CTH93 vaccines induced protection at day 3 post infection (FIG. 13E). This was in contrast to CTH93 vaccinated mice which induced no significant protection at day 3 post infection. At day 7 post infection both vaccines including the strong T cell target (CTH93) induced a significant level of protection (FIGS. 13D&E).

### Conclusions

We were able to mix the heterologous VD4 repeats with strong T cell antigens without the loss of in vitro neutralization and early in vivo protection against a Serovar D challenge. Moreover, the mix of B and T cell targets induced an efficient CMI mediated clearance of residual organisms leading to high levels of protection at day 7 post infection.

### Example 10: Testing the Effect of Different Adjuvant Systems

### Introduction

In order to investigate if the high antibody response against heterologous immuno-repeats were only seen when the vaccine were administered in CAF01-we compared the antibody response and the neutralization titer after immunizing with CTH527 (S vE VD4^{ext})*4 in CAF01 or Alum.

### Results

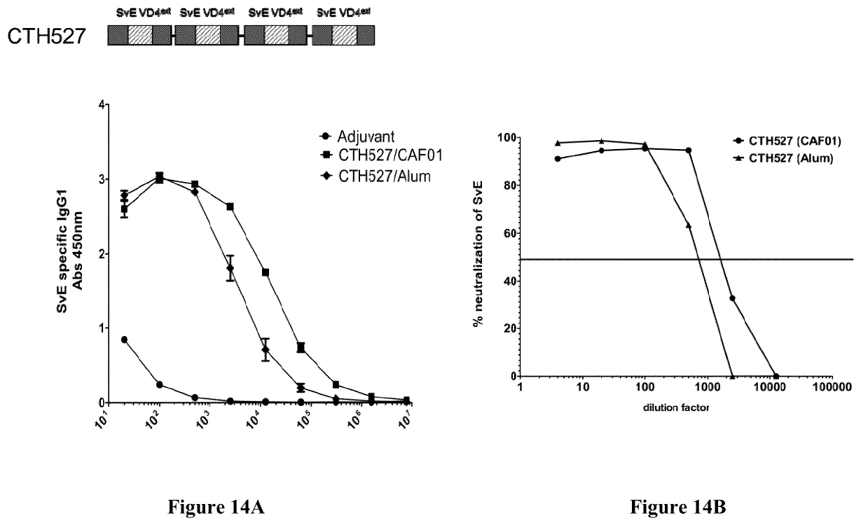

Both adjuvant systems induced a high antibody response against the surface of S vE when administered together with CTH527 (FIG. 14A), and the antibodies from both groups were able to neutralize S vE in vitro (FIG. 14B).

### Example 11: Vaccination with Heterologous Immuno-Repeats Composed of Reduced Length of the VD4^{ext} Regions from S vD, S vE, S vF and S vG

### Introduction

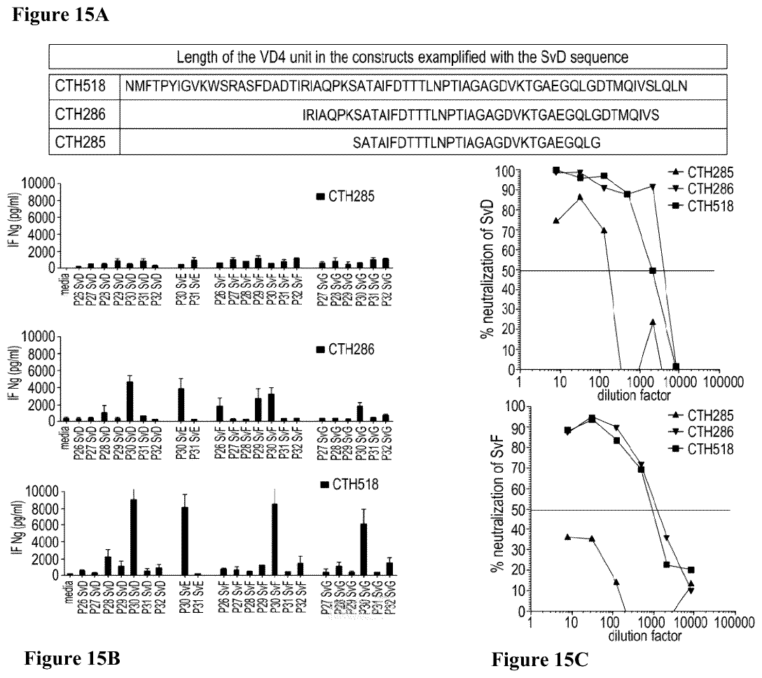

We next compared heterologous immuno-repeat constructs composed of reduced length of the VD4 region (CTH285 (SEQ ID NO: 69) and CTH286 (SEQ ID NO: 70)) compared to the CTH518 construct (CTH518 (SEQ ID NO: 53)) (FIG. 15A).

### Results

4 mice/group were immunized 3 times with 14 days between immunizations. The vaccines were emulsified in CAF01 and administered simultaneously by the subcutaneous (sc, 5 μg) and intranasal (i.n, 5 μg) routes. Splenocytes from 4 mice/group were isolated and the T cell responses to overlapping peptides representing the VD4^{ext} region (FIG. 15B) and the capacity of the serum to neutralize a serovar D and F infection (FIG. 15C) were investigated. Much reduced levels of VD4 T cell recognition, and neutralization was seen after vaccination with CTH285 where the VD4^{ext} regions from the different serovars were reduced with 38 aa. CTH286 on the other hand (each VD4^{ext} region reduced with 24 aa) induced similar levels of T cell responses and had the same capacity to neutralize a serovar D infection as CTH518.

### Conclusion

We demonstrated that by reducing the length of the VD4^{ext} regions with 38 aa we reduced both the T cell responses and the capacity to neutralize a serovar D and F infection.

### Example 12: Vaccination with Heterologous Immuno-Repeats Composed of Extended VD4^{ext} Regions from S vD, S vE, S vF, S vG, S vIa and S vJ

### Introduction

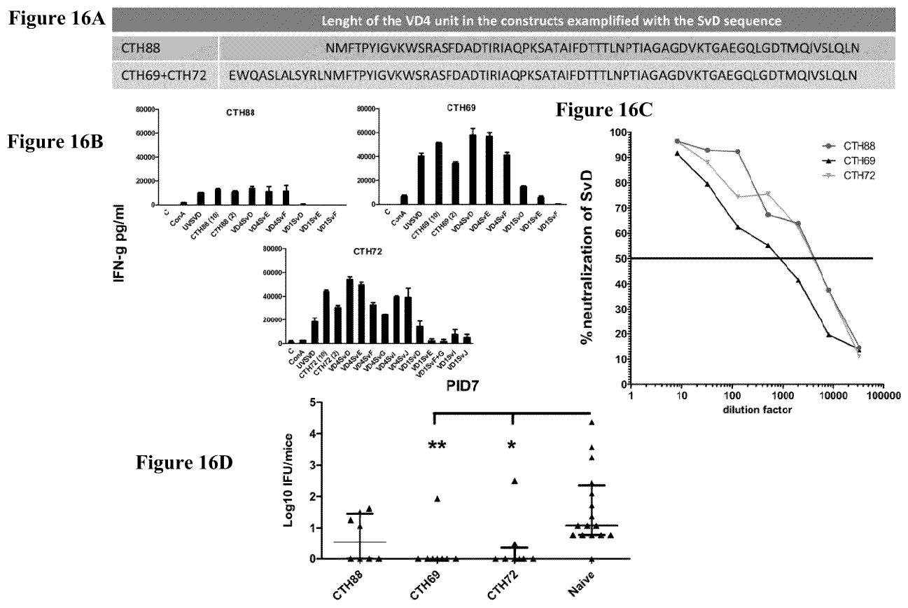

We next investigated if we by extending the length of the VD4^{ext} region could enhance the T cell response to the immuno-repeat constructs. We designed two constructs CTH69 (SEQ ID NO: 47) and CTH72 (SEQ ID NO: 48) (FIG. 16A). CTH69 was similar to CTH88 but the VD4^{ext} regions from S vD, S vE and S vF was extended by 12aa N-terminally (FIG. 16B). CTH72 also contained VD1 and VD4^{ext} regions from S vG, S vIa and S vJ.

### Results

Mice were immunized 3 times with 14 days between immunizations. The vaccines were emulsified in CAF01 and administered simultaneously by the subcutaneous (sc, 5 μg) and intranasal (i.n, 5 μg) routes. T cell responses to the antigen used for immunization and to peptide pools representing the VD1 and VD4 regions from the different serovars were investigated (FIGS. 16A-D). Extending the VD4^{ext} regions induced a significant higher T cell response (>40.000 μg/ml) compared to the T cell response obtained with CTH88 (<20.000 pg/ml) (FIG. 16B). Importantly, both of the extended constructs were still able to neutralize a serovar D infection in vitro (FIG. 16C). Comparing the protective efficacy of the vaccines, CTH69 and CTH72 induced a significant level of protection at day 7 post infection which could possibly be explained by the stronger T cell response induced by these vaccines compared to CTH88 (FIG. 16D).

### Conclusion

Extending the VD4^{ext} region enhanced the T cell response compared to CTH88 which led to enhanced protection at day 7 post infection.

### REFERENCES

1. WHO. Global Prevalence and Incidence of selected Curable Sexually Transmitted Infections: Overview and Estimates. World Health Organization, Geneva, Switzerland; 2001.
2. Paavonen J, Eggert-Kruse W. Chlamydia trachomatis: impact on human reproduction. Hum Reprod Update 1999, 5 (5): 433-447.
3. Plummer F A, Simonsen J N, Cameron D W, Ndinya-Achola J O, Kreiss J K, Gakinya M N, et al. Cofactors in male-female sexual transmission of human immunodeficiency virus type 1. J Infect Dis 1991, 163 (2): 233-239.
4. Anttila T, Saikku P, Koskela P, Bloigu A, Dillner J, Ikaheimo I, et al. Serotypes of Chlamydia trachomatis and risk for development of cervical squamous cell carcinoma. Jama 2001, 285 (1): 47-51.
5. Golden M R, Schillinger J A, Markowitz L, St Louis M E. Duration of untreated genital infections with Chlamydia trachomatis: a review of the literature. Sex Transm Dis 2000, 27 (6): 329-337.
6. Batteiger B E, Xu F, Johnson R E, Rekart M L. Protective immunity to Chlamydia trachomatis genital infection: evidence from human studies. J Infect Dis, 201 Suppl 2: S178-189.
7. Brunham R C, Rey-Ladino J. Immunology of Chlamydia infection: implications for a Chlamydia trachomatis vaccine. Nat Rev Immunol 2005, 5 (2): 149-161.
8. Su H, Caldwell H D. CD4+ T cells play a significant role in adoptive immunity to Chlamydia trachomatis infection of the mouse genital tract. Infect Immun 1995, 63 (9): 3302-3308.
9. Morrison S G, Su H, Caldwell H D, Morrison R P. Immunity to murine Chlamydia trachomatis genital tract reinfection involves B cells and CD4 (+) T cells but not CD8 (+) T cells. Infect Immun 2000, 68 (12): 6979-6987.
10. Morrison R P, Caldwell H D. Immunity to murine chlamydial genital infection. Infect Immun 2002, 70 (6): 2741-2751.
11. Rasmussen S J. Chlamydia immunology. Curr Opin Infect Dis 1998, 11 (1): 37-41.
12. Rank R. In: Chlamydia Intracellular Biology, Pathogenesis and Immunity. Washington D C. ASM Press 1999: Pp. 239-296.
13. Morrison S G, Morrison R P. Resolution of secondary Chlamydia trachomatis genital tract infection in immune mice with depletion of both CD4+ and CD8+ T cells. Infect Immun 2001, 69 (4): 2643-2649.
14. Moore T, Ekworomadu C O, Eko F O, MacMillan L, Ramey K, A nanaba G A, et al. Fc receptor-mediated antibody regulation of T cell immunity against intracellular pathogens. J Infect Dis 2003, 188 (4): 617-624.
15. Pal S, Rangel J, Peterson E M, de la Maza L M. Immunogenic and protective ability of the two developmental forms of Chlamydiae in a mouse model of infertility. Vaccine 1999, 18 (7-8): 752-761.
16. Darville T, Hiltke T J. Pathogenesis of genital tract disease due to Chlamydia trachomatis. J Infect Dis 2010, 201 Suppl 2: S114-125.
17. Hansen J, Jensen K T, Follmann F, Agger E M, Theisen M, Andersen P. Liposome Delivery of Chlamydia muridarum Major Outer Membrane Protein Primes a Th1 Response That Protects against Genital Chlamydial Infection in a Mouse Model. J Infect Dis 2008, 198 (5): 758-767.
18. Olsen A W, Theisen M, Christensen D, Follmann F, Andersen P. Protection against Chlamydia promoted by a subunit vaccine (CTH1) compared with a primary intranasal infection in a mouse genital challenge model. PLOS One, 5 (5): e10768.
19. Li W, Murthy A K, Guentzel M N, Chambers J P, Forsthuber T G, Seshu J, et al. Immunization with a combination of integral chlamydial antigens and a defined secreted protein induces robust immunity against genital chlamydial challenge. Infect Immun 2010, 78 (9): 3942-3949.
20. Olsen A W, Follmann F, Hojrup P, Leah R, Sand C, Andersen P, et al. Identification of human T-cell targets recognized during the Chlamydia trachomatis genital infection. J Infect Dis 2007, 196:1546-1552.
21. Olsen A W, Follmann F, Jensen K, Hojrup P, Leah R, Sorensen H, et al. Identification of C T 521 as a frequent target of Th1 cells in patients with urogenital Chlamydia trachomatis infection. J Infect Dis 2006, 194 (9): 1258-1266.
22. Follmann F, Olsen A W, Jensen K T, Hansen P R, Andersen P, Theisen M. Antigenic profiling of a Chlamydia trachomatis gene-expression library. J Infect Dis 2008, 197 897-905.
23. Sharma J, Zhong Y, Dong F, Piper J M, Wang G, Zhong G. Profiling of human antibody responses to Chlamydia trachomatis urogenital tract infection using microplates arrayed with 156 chlamydial fusion proteins. Infect Immun 2006, 74 (3): 1490-1499.
24. Coler R N, Bhatia A, Maisonneuve J F, Probst P, Barth B, Ovendale P, et al. Identification and characterization of novel recombinant vaccine antigens for immunization against genital Chlamydia trachomatis. FEMS Immunol Med Microbiol 2009, 55 (2): 258-270.
25. Karunakaran K P, Rey-Ladino J, Stoynov N, Berg K, Shen C, Jiang X, et al. Immunoproteomic discovery of novel T cell antigens from the obligate intracellular pathogen Chlamydia. J Immunol 2008, 180 (4): 2459-2465.
26. Yu H, Jiang X, Shen C, Karunakaran K P, Brunham R C. Novel Chlamydia muridarum T cell antigens induce protective immunity against lung and genital tract infection in murine models. J Immunol 2009, 182 (3): 1602-1608.
27. Molina D M, Pal S, Kayala M A, Teng A, Kim P J, Baldi P, et al. Identification of immunodominant antigens of Chlamydia trachomatis using proteome microarrays. Vaccine 2010, 28 (17): 3014-3024.
28. Stephens R S, Kalman S, Lammel C, Fan J, Marathe R, Aravind L, et al. Genome sequence of an obligate intracellular pathogen of humans: Chlamydia trachomatis. Science 1998, 282 (5389): 754-759.
29. Sette A, Rappuoli R. Reverse vaccinology: developing vaccines in the era of genomics. Immunity 2010, 33 (4): 530-541.
30. Igietseme J U, Eko F O, Black C M. Chlamydia vaccines: recent developments and the role of adjuvants in future formulations. Expert Rev Vaccines 2011, 10 (11): 1585-1596.
31. Rockey D D, Wang J, Lei L, Zhong G. Chlamydia vaccine candidates and tools for chlamydial antigen discovery. Expert Rev Vaccines 2009, 8 (10): 1365-1377.
32. Farris C M, Morrison R P. Vaccination against Chlamydia genital infection utilizing the murine C. muridarum model. Infect Immun 2011, 79 (3): 986-996.
33. Kubo A, Stephens R S. Characterization and functional analysis of PorB, a Chlamydia porin and neutralizing target. Mol Microbiol 2000, 38 (4): 772-780.
34. Kawa D E, Schachter J, Stephens R S. Immune response to the Chlamydia trachomatis outer membrane protein PorB. Vaccine 2004, 22 (31-32): 4282-4286.
35. Crane D D, Carlson J H, Fischer E R, Bavoil P, Hsia R C, Tan C, et al. Chlamydia trachomatis polymorphic membrane protein D is a species-common pan-neutralizing antigen. Proc Natl Acad Sci USA 2006, 103 (6): 1894-1899.
36. Baehr W, Zhang Y X, Joseph T, Su H, Nano F E, Everett K D, et al. Mapping antigenic domains expressed by Chlamydia trachomatis major outer membrane protein genes. Proc Natl Acad Sci USA 1988, 85 (11): 4000-4004.
37. Bavoil P, Ohlin A, Schachter J. Role of disulfide bonding in outer membrane structure and permeability in Chlamydia trachomatis. Infect Immun 1984, 44 (2): 479-485.
38. Hatch T P, Allan I, Pearce J H. Structural and polypeptide differences between envelopes of infective and reproductive life cycle forms of Chlamydia spp.) Bacteriol 1984, 157 (1): 13-20.
39. Stephens R S, Sanchez-Pescador R, Wagar E A, Inouye C, Urdea M S. Diversity of Chlamydia trachomatis major outer membrane protein genes. J Bacteriol 1987, 169 (9): 3879-3885.
40. Caldwell H D, Perry L J. Neutralization of Chlamydia trachomatis infectivity with antibodies to the major outer membrane protein. Infect Immun 1982, 38 (2): 745-754.
41. Peeling R, Maclean I W, Brunham R C. In vitro neutralization of Chlamydia trachomatis with monoclonal antibody to an epitope on the major outer membrane protein. Infect Immun 1984, 46 (2): 484-488.
42. Zhang Y X, Stewart S, Joseph T, Taylor H R, Caldwell H D. Protective monoclonal antibodies recognize epitopes located on the major outer membrane protein of Chlamydia trachomatis. J Immunol 1987, 138 (2): 575-581.
43. Zhang Y X, Stewart S J, Caldwell H D. Protective monoclonal antibodies to Chlamydia trachomatis serovar- and serogroup-specific major outer membrane protein determinants. Infect Immun 1989, 57 (2): 636-638.
44. Cotter T W, Meng Q, Shen Z L, Zhang Y X, Su H, Caldwell H D. Protective efficacy of major outer membrane protein-specific immunoglobulin A (IgA) and IgG monoclonal antibodies in a murine model of Chlamydia trachomatis genital tract infection. Infect Immun 1995, 63 (12): 4704-4714.
45. Bandea C I, Debattista J, Joseph K, Igietseme J, Timms P, Black C M. Chlamydia trachomatis serovars among strains isolated from members of rural indigenous communities and urban populations in Australia. J Clin Microbiol 2008, 46 (1): 355-356.
46. Hsu M C, Tsai P Y, Chen K T, Li L H, Chiang C C, Tsai J J, et al. Genotyping of Chlamydia trachomatis from clinical specimens in Taiwan. J Med Microbiol 2006, 55 (Pt 3): 301-308.
47. Jonsdottir K, Kristjansson M, Hjaltalin Olafsson J, Steingrimsson O. The molecular epidemiology of genital Chlamydia trachomatis in the greater Reykjavik area, Iceland. Sex Transm Dis 2003, 30 (3): 249-256.
48. Lysen M, Osterlund A, Rubin C J, Persson T, Persson I, Herrmann B. Characterization of ompA genotypes by sequence analysis of DNA from all detected cases of Chlamydia trachomatis infections during 1 year of contact tracing in a Swedish County. J Clin Microbiol 2004, 42 (4): 1641-1647.
49. Millman K, Black C M, Johnson R E, Stamm W E, Jones R B, Hook E W, et al. Population-based genetic and evolutionary analysis of Chlamydia trachomatis urogenital strain variation in the United States. J Bacteriol 2004, 186 (8): 2457-2465.
50. Millman K, Black C M, Stamm W E, Jones R B, Hook E W, 3rd, Martin D H, et al. Population-based genetic epidemiologic analysis of Chlamydia trachomatis serotypes and lack of association between ompA polymorphisms and clinical phenotypes. Microbes Infect 2006, 8 (3): 604-611.
51. Su H, Parnell M, Caldwell H D. Protective efficacy of a parenterally administered MOM P-derived synthetic oligopeptide vaccine in a murine model of Chlamydia trachomatis genital tract infection: serum neutralizing IgG antibodies do not protect against chlamydial genital tract infection. Vaccine 1995, 13 (11): 1023-1032.
52. Pal S, Barnhart K M, Wei Q, A bai A M, Peterson E M, de la Maza L M. Vaccination of mice with DNA plasmids coding for the Chlamydia trachomatis major outer membrane protein elicits an immune response but fails to protect against a genital challenge. Vaccine 1999, 17 (5): 459-465.
53. Zhang D J, Yang X, Shen C, Brunham R C. Characterization of immune responses following intramuscular DNA immunization with the MOM P gene of Chlamydia trachomatis mouse pneumonitis strain. Immunology 1999, 96 (2): 314-321.
54. Pal S, Theodor I, Peterson E M, de la Maza L M. Immunization with the Chlamydia trachomatis mouse pneumonitis major outer membrane protein can elicit a protective immune response against a genital challenge. Infect Immun 2001, 69 (10): 6240-6247.
55. Shaw J, Grund V, Durling L, Crane D, Caldwell H D. Dendritic cells pulsed with a recombinant chlamydial major outer membrane protein antigen elicit a CD4 (+) type 2 rather than type 1 immune response that is not protective. Infect Immun 2002, 70 (3): 1097-1105.
56. Kari L, Whitmire W M, Crane D D, Reveneau N, Carlson J H, Goheen M M, et al. Chlamydia trachomatis native major outer membrane protein induces partial protection in nonhuman primates: implication for a trachoma transmission-blocking vaccine. J Immunol 2009, 182 (12): 8063-8070.
57. Carmichael J R, Pal S, Tifrea D, de la Maza L M. Induction of protection against vaginal shedding and infertility by a recombinant Chlamydia vaccine. Vaccine 2011, 29 (32): 5276-5283.
58. Y en T Y, Pal S, de la Maza L M. Characterization of the disulfide bonds and free cysteine residues of the Chlamydia trachomatis mouse pneumonitis major outer membrane protein. Biochemistry 2005, 44 (16): 6250-6256.
59. Stephens R S, Wagar E A, Schoolnik G K. High-resolution mapping of serovar-specific and common antigenic determinants of the major outer membrane protein of Chlamydia trachomatis. J Exp Med 1988, 167 (3): 817-831.
60. Murdin A D, Su H, Klein M H, Caldwell H D. Poliovirus hybrids expressing neutralization epitopes from variable domains I and I V of the major outer membrane protein of Chlamydia trachomatis elicit broadly cross-reactive C. trachomatis-neutralizing antibodies. Infect Immun 1995, 63 (3): 1116-1121.
61. Murdin A D, Su H, Manning D S, Klein M H, Parnell M J, Caldwell H D. A poliovirus hybrid expressing a neutralization epitope from the major outer membrane protein of Chlamydia trachomatis is highly immunogenic. Infect Immun 1993, 61 (10): 4406-4414.
62. Villeneuve A, Brossay L, Paradis G, Hebert J. Determination of neutralizing epitopes in variable domains I and I V of the major outer-membrane protein from Chlamydia trachomatis serovar K. Microbiology 1994, 140 (Pt 9): 2481-2487.
63. Villeneuve A, B rossay L, Paradis G, Hebert J. Characterization of the humoral response induced by a synthetic peptide of the major outer membrane protein of Chlamydia trachomatis serovar B. Infect Immun 1994, 62 (8): 3547-3549.
64. Motin V L, de la Maza L M, Peterson E M. Immunization with a peptide corresponding to chlamydial heat shock protein 60 increases the humoral immune response in C3H mice to a peptide representing variable domain 4 of the major outer membrane protein of Chlamydia trachomatis. Clin Diagn Lab Immunol 1999, 6 (3): 356-363.
65. Su H, Caldwell H D. Immunogenicity of a synthetic oligopeptide corresponding to antigenically common T-helper and B-cell neutralizing epitopes of the major outer membrane protein of Chlamydia trachomatis. Vaccine 1993, 11 (11): 1159-1166.
66. Toye B, Zhong G M, Peeling R, Brunham R C. Immunologic characterization of a cloned fragment containing the species-specific epitope from the major outer membrane protein of Chlamydia trachomatis. Infect Immun 1990, 58 (12): 3909-3913.
67. Mygind P, Christiansen G, Persson K, Birkelund S. Detection of Chlamydia trachomatis-specific antibodies in human sera by recombinant major outer-membrane protein polyantigens. J Med Microbiol 2000, 49 (5): 457-465.
68. Qu Z, Cheng X, de la Maza L M, Peterson E M. Analysis of the humoral response elicited in mice by a chimeric peptide representing variable segments I and I V of the major outer membrane protein of Chlamydia trachomatis. Vaccine 1994, 12 (6): 557-564.
69. Peterson E M, Cheng X, Qu Z, de la Maza L M. The effect of orientation within a chimeric peptide on the immunogenicity of Chlamydia trachomatis epitopes. Mol Immunol 1996, 33 (4-5): 335-339.
70. Caldwell H D, Kromhout J, Schachter J. Purification and partial characterization of the major outer membrane protein of Chlamydia trachomatis. Infect Immun 1981, 31 (3): 1161-1176.
71. Ravn P, Demissie A, Eguale T, Wondwosson H, Lein D, A moudy H A, et al. Human T cell responses to the ESAT-6 antigen from Mycobacterium tuberculosis. J Infect Dis 1999, 179 (3): 637-645.
72. Stryhn A, Pedersen L O, Romme T, Holm C B, Holm A, Buus S. Peptide binding specificity of major histocompatibility complex class I resolved into an array of apparently independent subspecificities: quantitation by peptide libraries and improved prediction of binding. Eur J Immunol 1996, 26 (8): 1911-1918.
73. Harboe M, Oettinger T, Wiker H G, Rosenkrands I, Andersen P. Evidence for occurrence of the ESAT-6 protein in Mycobacterium tuberculosis and virulent Mycobacterium bovis and for its absence in Mycobacterium bovis BCG. Infect Immun 1996, 64 (1): 16-22.
74. Volp K, Mathews S, Timms P, Hafner L. Peptide immunization of guinea pigs against Chlamydia psittaci (GPIC agent) infection induces good vaginal secretion antibody response, in vitro neutralization and partial protection against live challenge. Immunol Cell Biol 2001, 79 (3): 245-250.
75. Hinton H J, Jegerlehner A, Bachmann M F. Pattern recognition by B cells: the role of antigen repetitiveness versus Toll-like receptors. Current topics in microbiology and immunology 2008, 319:1-15.
76. Kim S K, DeM ars R. Epitope clusters in the major outer membrane protein of Chlamydia trachomatis. Curr Opin Immunol 2001, 13 (4): 429-436.
77. Findlay H E, McClafferty H, Ashley R H. Surface expression, single-channel analysis and membrane topology of recombinant Chlamydia trachomatis Major Outer Membrane Protein. BMC Microbiol 2005, 5:5.
78. Cobbold S P, Jayasuriya A, Nash A, Prospero T D, Waldmann H. Therapy with monoclonal antibodies by elimination of T-cell subsets in vivo. Nature 1984, 312 (5994): 548-551.
79. Qin S, Cobbold S, Tighe H, Benjamin R, Waldmann H. CD4 monoclonal antibody pairs for immunosuppression and tolerance induction. Eur J Immunol 1987, 17 (8): 1159-1165.

U.S. patent application Ser. No. 15/956,731, filed Apr. 18, 2018, U.S. patent application Ser. No. 14/216,403, filed Mar. 17, 2014, U.S. Provisional Patent Application No. 61/802,907, filed Mar. 18, 2013, Danish Patent Application Nos. PA 2013 00155, filed Mar. 18, 2013, and PA 2013 00684, Dec. 11, 2013, including sequence listings, are incorporated herein by reference in their entireties.

1.-22. (canceled)

23. A nucleic acid encoding a polypeptide comprising 3 or more immuno-repeat units of surface exposed fragments of the major outer membrane protein (MOMP), wherein each immuno-repeat unit comprises an amino acid sequence which comprises
i) the variable domain 4 (VD4) region; or
ii) the variable domain 1 (VD1) region, or
iii) both i) and ii) of the MOMP from a serotype of a Chlamydia species selected from Chlamydia trachomatis, Chlamydia pneumoniae, Chlamydia muridarum, Chlamydia suis, and Chlamydiaphila abortus.

24. The nucleic acid according to claim 23, wherein the amino acid sequences are linearized.

25. The nucleic acid according to claim 23, wherein the immune-repeats are homologous.

26. The nucleic acid according to claim 23, wherein the amino acid sequences comprising the VD4 region, the VD1 region or both regions of the MOMP are placed next to each other.

27. The nucleic acid according to claim 23, wherein the immune-repeats are heterologous.

28. The nucleic acid according to claim 23, wherein the amino acid sequences comprising the VD4 region, the VD1 region or both regions of the MOMP are spaced with a linker.

29. The nucleic acid according to claim 23, wherein the MOMP is from a serotype of Chlamydia pneumoniae or serotype D, E, F, G, Ia or J of Chlamydia trachomatis.

30. The nucleic acid according to claim 23, further comprising one or more of variable domain 2 (VD2) and variable domain 3 (VD3) of the MOMP from any serotype of said Chlamydia species.

31. The nucleic acid according to claim 23, comprising an amino acid sequence defined in formula I:
xx₁-VD4-xx₂ (Formula I) wherein
VD4 is independently selected from SEQ ID NO: 15-20 or an amino acid which has at least 80% sequence identity with SEQ ID NO: 15-20,
and
xx₁ consists of
i) the amino acid sequence EWQASLALSYRLNMFTPYIGVKWSRASFDADTIRIAQPK (SEQ ID NO: 21) or
ii) a subsequence of the amino acid sequence in i), said subsequence comprising 1-38 amino acid residues, starting with the C-terminal K in the amino acid sequence in i)
and
xx₂ consists of
iii) the amino acid sequence DTMQIVSLQLNKMKSRKSCGIAVGTTIVDA (SEQ ID NO: 22) or
iv) a subsequence of the amino acid sequence in iii), said subsequence comprising 1-29 amino acid residues, starting with the N-terminal D in the amino acid sequence in iii).

32. The nucleic acid according to claim 23, comprising an amino acid sequence selected from SEQ ID NO: 23-28 and 49-59.

33. The nucleic acid according to claim 23, comprising an amino acid sequence defined in formula II:
yy₁-VD1-yy₂ (Formula II) wherein
VD1 is independently selected from SEQ ID NO: 1-6,
and
yy₁ consists of
i) the amino acid sequence DAISMRVGYYGDFVFDRVLKTDVNKEFQMG (SEQ ID NO: 7) or
ii) a subsequence of the amino acid sequence in i), said subsequence comprising 1-30 amino acid residues, starting with the C-terminal G in the amino acid sequence in i)
and
yy₂ consists of
iii) the amino acid sequence NPAYGRHMQDAEMFTNAA (SEQ ID NO: 8) or
iv) a subsequence of the amino acid sequence in iii), said subsequence comprising 1-18 amino acid residues, starting with the N-terminal N in the amino acid sequence in iii).

34. The nucleic acid according to claim 23, comprising an amino acid sequence selected from SEQ ID NO.: 9-14 and 45-48.

35. A nucleic acid encoding a polypeptide comprising more than one immuno-repeat unit of surface exposed fragments of variable domain 4 (VD4) in major outer membrane protein (MOMP) from a serotype of a Chlamydia or Chlamydiaphila species selected from Chlamydia trachomatis, Chlamydia pneumoniae, Chlamydia muridarum, Chlamydia suis, and Chlamydiaphila abortus, said polypeptide comprising the amino acid sequence selected from SEQ ID NO.: 23-28 and 49-59.

36. A nucleic acid encoding a polypeptide comprising more than one immuno-repeat unit of surface exposed fragments of variable domain 1 (VD1) in major outer membrane protein (MOMP) from a serotype of a Chlamydia or Chlamydiaphila species selected from Chlamydia trachomatis, Chlamydia pneumoniae, Chlamydia muridarum, Chlamydia suis, and Chlamydiaphila abortus, said polypeptide comprising the amino acid sequence selected from SEQ ID NO.: 9-14 and 45-48.

37. The nucleic acid according to claim 23, wherein said polypeptide further comprises a moiety that facilitates export of the polypeptide when produced recombinantly, a moiety that facilitates purification of the fusion protein, or a moiety which enhances immunogenicity.

38. The nucleic acid according to claim 36, wherein said moiety which enhances immunogenicity is a T-cell target selected from Chlamydia trachomatis (Ct) antigens CT043, CT004, CT414, and CT681.

39. The nucleic acid sequence of claim 38, comprising an amino acid sequence selected from SEQ ID NOS: 60-68.

40. The nucleic acid according to claim 38, comprising SEQ ID NO: 64.

41. The nucleic acid according to claim 23, wherein said nucleic acid is DNA or RNA.

42. A pharmaceutical composition comprising the nucleic acid according to claim 23 and one or more of a pharmacologically acceptable carrier, excipient, adjuvant, and immune modulator, wherein said nucleic acid is DNA or RNA.


SEQUENCE LISTING (255 sequences)

SEQ ID NO: 1 - protein, 20 aa, Chlamydia trachomatis
AKPTTDTGNSAAPSTLTARE

SEQ ID NO: 2 - protein, 20 aa, Chlamydia trachomatis
DKPTSTTGNATAPTTLTARE

SEQ ID NO: 3 - protein, 21 aa, Chlamydia trachomatis
EALAGASGNTTSTLSKLVERT

SEQ ID NO: 4 - protein, 21 aa, Chlamydia trachomatis
EALAGASGNTTSTLSKLVERT

SEQ ID NO: 5 - protein, 22 aa, Chlamydia trachomatis
AAPTTKDIAGLENDPTTNVARP

SEQ ID NO: 6 - protein, 22 aa, Chlamydia trachomatis
AAPTTSDVAGLQNDPTTNVARP

SEQ ID NO: 7 - protein, 30 aa, Chlamydia trachomatis
DAISMRVGYYGDFVFDRVLKTDVNKEFQMG

SEQ ID NO: 8 - protein, 18 aa, Chlamydia trachomatis
NPAYGRHMQDAEMFTNAA

SEQ ID NO: 9 - protein, 59 aa, Chlamydia trachomatis
AISMRVGYYGDFVFDRVLKTDVNKEFQMGAKPTTDTGNSAAPSTLTARENPAYGRHMQD

SEQ ID NO: 10 - protein, 59 aa, Chlamydia trachomatis
AISMRMGYYGDFVFDRVLKTDVNKEFQMGDKPTSTTGNATAPTTLTARENPAYGRHMQD

SEQ ID NO: 11 - protein, 60 aa, Chlamydia trachomatis
AISMRMGYYGDFVFDRVLKTDVNKEFEMGEALAGASGNTTSTLSKLVERTNPAYGKHMQD

SEQ ID NO: 12 - protein, 60 aa, Chlamydia trachomatis; identical to SEQ ID NO: 11

SEQ ID NO: 13 - protein, 61 aa, Chlamydia trachomatis
AISMRMGYYGDFVFDRVLKTDVNKEFQMGAAPTTKDIAGLENDPTTNVARPNPAYGKHMQD

SEQ ID NO: 14 - protein, 61 aa, Chlamydia trachomatis
AISMRMGYYGDFVFDRVLKTDVNKEFQMGAAPTTSDVAGLQNDPTTNVARPNPAYGKHMQD

SEQ ID NO: 15 - protein, 30 aa, Chlamydia trachomatis
SATAIFDTTTLNPTIAGAGDVKTGAEGQLG

SEQ ID NO: 16 - protein, 30 aa, Chlamydia trachomatis
SATAIFDTTTLNPTIAGAGDVKASAEGQLG

SEQ ID NO: 17 - protein, 31 aa, Chlamydia trachomatis
LVTPVVDITTLNPTIAGCGSVAGANTEGQIS

SEQ ID NO: 18 - protein, 31 aa, Chlamydia trachomatis
LAKPVVDITTLNPTIAGCGSVVAANSEGQIS

SEQ ID NO: 19 - protein, 31 aa, Chlamydia trachomatis
LAEAILDVTTLNPTIAGKGTVVASGSDNDLA

SEQ ID NO: 20 - protein, 31 aa, Chlamydia trachomatis
LAEAILDVTTLNPTIAGKGTVVASGSENDLA

SEQ ID NO: 21 - protein, 39 aa, Chlamydia trachomatis
EWQASLALSYRLNMFTPYIGVKWSRASFDADTIRIAQPK

SEQ ID NO: 22 - protein, 30 aa, Chlamydia trachomatis
DTMQIVSLQLNKMKSRKSCGIAVGTTIVDA

SEQ ID NO: 23 - protein, 68 aa, Chlamydia trachomatis
NMFTPYIGVKWSRASFDADTIRIAQPKSATAIFDTTTLNPTIAGAGDVKTGAEGQLGDTMQIVSLQLN

SEQ ID NO: 24 - protein, 68 aa, Chlamydia trachomatis
NMFTPYIGVKWSRASFDADTIRIAQPKSATAIFDTTTLNPTIAGAGDVKASAEGQLGDTMQIVSLQLN

SEQ ID NO: 25 - protein, 69 aa, Chlamydia trachomatis
NMFTPYIGVKWSRASFDSDTIRIAQPRLVTPVVDITTLNPTIAGCGSVAGANTEGQISDTMQIVSLQLN

SEQ ID NO: 26 - protein, 69 aa, Chlamydia trachomatis
NMFTPYIGVKWSRASFDSNTIRIAQPKLAKPVVDITTLNPTIAGCGSVVAANSEGQISDTMQIVSLQLN

SEQ ID NO: 27 - protein, 69 aa, Chlamydia trachomatis
NMFTPYIGVKWSRVSFDADTIRIAQPKLAEAILDVTTLNPTIAGKGTVVASGSDNDLADTMQIVSLQLN

SEQ ID NO: 28 - protein, 69 aa, Chlamydia trachomatis
NMFTPYIGVKWSRVSFDADTIRIAQPKLAEAILDVTTLNPTIAGKGTVVASGSENDLADTMQIVSLQLN

SEQ ID NO: 29 - protein, 21 aa, Chlamydia trachomatis
DNENQKTVKAESVPNMSFDQS

SEQ ID NO: 30 - protein, 21 aa, Chlamydia trachomatis
DNENQSTVKTNSVPNMSLDQS

SEQ ID NO: 31 - protein, 21 aa, Chlamydia trachomatis
DGVNATKPAADSIPNVQLNQS

SEQ ID NO: 32 - protein, 21 aa, Chlamydia trachomatis
DSENATQPAATSIPNVQLNQS

SEQ ID NO: 33 - protein, 22 aa, Chlamydia trachomatis
TKTQSSNFNTAKLIPNAALNQA

SEQ ID NO: 34 - protein, 22 aa, Chlamydia trachomatis
TKTQASSFNTANLFPNTALNQA

SEQ ID NO: 35 - protein, 23 aa, Chlamydia trachomatis
TLGATSGYLKGNSASFNLVGLFG

SEQ ID NO: 36 - protein, 22 aa, Chlamydia trachomatis
VVELYTDTTFAWSVGARAALWE

SEQ ID NO: 37 - protein, 14 aa, Chlamydia trachomatis
KEFPLDLTAGTDAA

SEQ ID NO: 38 - protein, 14 aa, Chlamydia trachomatis
QEFPLALIAGTDAA

SEQ ID NO: 39 - protein, 14 aa, Chlamydia trachomatis
KEFPLDLTAGTDAA

SEQ ID NO: 40 - protein, 14 aa, Chlamydia trachomatis
QEFPLALTAGTDAA

SEQ ID NO: 41 - protein, 14 aa, Chlamydia trachomatis
AEFPLDITAGTEAA

SEQ ID NO: 42 - protein, 14 aa, Chlamydia trachomatis
AEFPLDITAGTEAA

SEQ ID NO: 43 - protein, 38 aa, Chlamydia trachomatis
ATLGASFQYAQSKPKVEELNVLCNAAEFTINKPKGYVG

SEQ ID NO: 44 - protein, 35 aa, Chlamydia trachomatis
TGTKDASIDYHEWQASLALSYRLNMFTPYIGVKWS

SEQ ID NO: 45 - protein, 127 aa, Chlamydia trachomatis
AISMRVGYYGDFVFDRVLKTDVNKEFQMGAKPTTDTGNSAAPSTLTARENPAYGRHMQDNMFTPYIGVKWSRASFDADTIRIAQPKSATAIFDTTTLNPTIAGAGDVKTGAEGQLGDTMQIVSLQLN

SEQ ID NO: 46 - protein, 383 aa, Chlamydia trachomatis
AISMRVGYYGDFVFDRVLKTDVNKEFQMGAKPTTDTGNSAAPSTLTARENPAYGRHMQDNMFTPYIGVKWSRASFDADTIRIAQPKSATAIFDTTTLNPTIAGAGDVKTGAEGQLGDTMQIVSLQLNAISMRMGYYGDFVFDRVLKTDVNKEFQMGDKPTSTTGNATAPTTLTARENPAYGRHMQDNMFTPYIGVKWSRASFDADTIRIAQPKSATAIFDTTTLNPTIAGAGDVKASAEGQLGDTMQIVSLQLNAISMRMGYYGDFVFDRVLKTDVNKEFEMGEALAGASGNTTSTLSKLVERTNPAYGKHMQDNMFTPYIGVKWSRASFDSDTIRIAQPRLVTPVVDITTLNPTIAGSGSVAGANTEGQISDTMQIVSLQLN

SEQ ID NO: 47 - protein, 419 aa, Chlamydia trachomatis
AISMRVGYYGDFVFDRVLKTDVNKEFQMGAKPTTDTGNSAAPSTLTARENPAYGRHMQDEWQASLALSYRLNMFTPYIGVKWSRASFDADTIRIAQPKSATAIFDTTTLNPTIAGAGDVKTGAEGQLGDTMQIVSLQLNAISMRMGYYGDFVFDRVLKTDVNKEFQMGDKPTSTTGNATAPTTLTARENPAYGRHMQDEWQASLALSYRLNMFTPYIGVKWSRASFDADTIRIAQPKSATAIFDTTTLNPTIAGAGDVKASAEGQLGDTMQIVSLQLNAISMRMGYYGDFVFDRVLKTDVNKEFEMGEALAGASGNTTSTLSKLVERTNPAYGKHMQDEWQASLSLSYRLNMFTPYIGVKWSRASFDSDTIRIAQPRLVTPVVDITTLNPTIAGSGSVAGANTEGQISDTMQIVSLQLN

SEQ ID NO: 48 - protein, 844 aa, Chlamydia trachomatis
AISMRVGYYGDFVFDRVLKTDVNKEFQMGAKPTTDTGNSAAPSTLTARENPAYGRHMQDEWQASLALSYRLNMFTPYIGVKWSRASFDADTIRIAQPKSATAIFDTTTLNPTIAGAGDVKTGAEGQLGDTMQIVSLQLNAISMRMGYYGDFVFDRVLKTDVNKEFQMGDKPTSTTGNATAPTTLTARENPAYGRHMQDEWQASLALSYRLNMFTPYIGVKWSRASFDADTIRIAQPKSATAIFDTTTLNPTIAGAGDVKASAEGQLGDTMQIVSLQLNAISMRMGYYGDFVFDRVLKTDVNKEFEMGEALAGASGNTTSTLSKLVERTNPAYGKHMQDEWQASLSLSYRLNMFTPYIGVKWSRASFDSDTIRIAQPRLVTPVVDITTLNPTIAGSGSVAGANTEGQISDTMQIVSLQLNAISMRMGYYGDFVFDRVLKTDVNKEFEMGEALAGASGNTTSTLSKLVERTNPAYGKHMQDEWQASLSLSYRLNMFTPYIGVKWSRASFDSNTIRIAQPKLAKPVVDITTLNPTIAGSGSVVAANSEGQISDTMQIVSLQLNAISMRMGYYGDFVFDRVLKTDVNKEFQMGAAPTTKDIAGLENDPTTNVARPNPAYGKHMQDEWQASLALSYRLNMFTPYIGVKWSRVSFDADTIRIAQPKLAEAILDVTTLNPTIAGKGTVVASGSDNDLADTMQIVSLQLNAISMRMGYYGDFVFDRVLKTDVNKEFQMGAAPTTSDVAGLQNDPTTNVARPNPAYGKHMQDEWQASLALSYRLNMFTPYIGVKWSRVSFDADTIRIAQPKLAEAILDVTTLNPTIAGKGTVVASGSENDLADTMQIVSLQLN

SEQ ID NO: 49 - protein, 205 aa, Chlamydia trachomatis
NMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLN

SEQ ID NO: 50 - protein, 75 aa, Chlamydia trachomatis
MHHHHHHNMFTPYIGVKWSRASFDADTIRIAQPKSATAIFDTTTLNPTIAGAGDVKASAEGQLGDTMQIVSLQLN

SEQ ID NO: 51 - protein, 69 aa, Chlamydia trachomatis; identical to SEQ ID NO: 25

SEQ ID NO: 52 - protein, 76 aa, Chlamydia trachomatis
MHHHHHHNMFTPYIGVKWSRASFDSDTIRIAQPRLVTPVVDITTLNPTIAGCGSVAGANTEGQISDTMQIVSLQLN

SEQ ID NO: 53 - protein, 274 aa, Chlamydia trachomatis
NMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 54 - protein, 322 aa, Chlamydia trachomatis
EWQASLALSYRLNMFTPYIGVKWSRASFDADTIRIAQPKSATAIFDTTTLNPTIAGAGDVKTGAEGQLGDTMQIVSLQLNEWQASLALSYRLNMFTPYIGVKWSRASFDADTIRIAQPKSATAIFDTTTLNPTIAGAGDVKASAEGQLGDTMQIVSLQLNEWQASLSLSYRLNMFTPYIGVKWSRASFDSDTIRIAQPRLVTPVVDITTLNPTIAGSGSVAGANTEGQISDTMQIVSLQLNEWQASLSLSYRLNMFTPYIGVKWSRASFDSNTIRIAQPKLAKPVVDITTLNPTIAGSGSVVAANSEGQISDTMQIVSLQLN

SEQ ID NO: 55 - protein, 484 aa, Chlamydia trachomatis
EWQASLALSYRLNMFTPYIGVKWSRASFDADTIRIAQPKSATAIFDTTTLNPTIAGAGDVKTGAEGQLGDTMQIVSLQLNEWQASLALSYRLNMFTPYIGVKWSRASFDADTIRIAQPKSATAIFDTTTLNPTIAGAGDVKASAEGQLGDTMQIVSLQLNEWQASLSLSYRLNMFTPYIGVKWSRASFDSDTIRIAQPRLVTPVVDITTLNPTIAGSGSVAGANTEGQISDTMQIVSLQLNEWQASLSLSYRLNMFTPYIGVKWSRASFDSNTIRIAQPKLAKPVVDITTLNPTIAGSGSVVAANSEGQISDTMQIVSLQLNEWQASLALSYRLNMFTPYIGVKWSRVSFDADTIRIAQPKLAEAILDVTTLNPTIAGKGTVVASGSDNDLADTMQIVSLQLNEWQASLALSYRLNMFTPYIGVKWSRVSFDADTIRIAQPKLAEAILDVTTLNPTIAGKGTVVASGSENDLADTMQIVSLQLN

SEQ ID NO: 56 - protein, 276 aa, Chlamydia trachomatis
NMFTPYIGVKWSRASFDSDTIRIAQPRLVTPVVDITTLNPTIAGSGSVAGANTEGQISDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSDTIRIAQPRLVTPVVDITTLNPTIAGSGSVAGANTEGQISDTMQIVSLQLN

SEQ ID NO: 57 - protein, 544 aa, Chlamydia trachomatis
NMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLN

SEQ ID NO: 58 - protein, 272 aa, Chlamydia trachomatis
NMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDADTIRIAQPKSATAIFDTTTLNPTIAGAGDVKASAEGQLGDTMQIVSLQLN

SEQ ID NO: 59 - protein, 276 aa, Chlamydia trachomatis
NMFTPYIGVKWSRASFDSDTIRIAQPRLVTPVVDITTLNPTIAGCGSVAGANTEGQISDTMQIVSLQLNNMFTPYIGVKWSRASFDSDTIRIAQPRLVTPVVDITTLNPTIAGCGSVAGANTEGQISDTMQIVSLQLNNMFTPYIGVKWSRASFDSDTIRIAQPRLVTPVVDITTLNPTIAGCGSVAGANTEGQISDTMQIVSLQLNNMFTPYIGVKWSRASFDSDTIRIAQPRLVTPVVDITTLNPTIAGCGSVAGANTEGQISDTMQIVSLQLN

SEQ ID NO: 60 - protein, 785 aa, Chlamydia trachomatis
SRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRATVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGAISMRVGYYGDFVFDRVLKTDVNKEFQMGAKPTTDTGNSAAPSTLTARENPAYGRHMQDNMFTPYIGVKWSRASFDADTIRIAQPKSATAIFDTTTLNPTIAGAGDVKTGAEGQLGDTMQIVSLQLNAISMRMGYYGDFVFDRVLKTDVNKEFQMGDKPTSTTGNATAPTTLTARENPAYGRHMQDNMFTPYIGVKWSRASFDADTIRIAQPKSATAIFDTTTLNPTIAGAGDVKASAEGQLGDTMQIVSLQLNAISMRMGYYGDFVFDRVLKTDVNKEFEMGEALAGASGNTTSTLSKLVERTNPAYGKHMQDNMFTPYIGVKWSRASFDSDTIRIAQPRLVTPVVDITTLNPTIAGSGSVAGANTEGQISDTMQIVSLQLN

SEQ ID NO: 61 - protein, 740 aa, Chlamydia trachomatis
SRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRATVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGDAISMRVGYYGDFVFDRVLKTDVNKEFQMGAKPTTDTGNSAAPSTLTARENPAYGRHMQDAEMFTNAASMALNIWDRFDVFSTLGATSGYLKGNSASFNLVGLFGDNENQKTVKAESVPNMSFDQSVVELYTDTTFAWSVGARAALWESGSATLGASFQYAQSKPKVEELNVLSNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSSGIAVGTTIVDADKYAVTVETRLIDERAAHVNAQFRF

SEQ ID NO: 62 - protein, 294 aa, Chlamydia trachomatis
DAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLN

SEQ ID NO: 63 - protein, 294 aa, Chlamydia trachomatis
DAISMRVGYYGDFVFDRVLKTDVNKEFQMGAKPTTDTGNSAAPSTLTARENPAYGRHMQDAEMFTNAASMALNIWDRFDVFSTLGATSGYLKGNSASFNLVGLFGDNENQKTVKAESVPNMSFDQSVVELYTDTTFAWSVGARAALWESGSATLGASFQYAQSKPKVEELNVLSNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLN

SEQ ID NO: 64 - protein, 500 aa, Chlamydia trachomatis
DAISMRVGYYGDFVFDRVLKTDVNKEFQMGAKPTTDTGNSAAPSTLTARENPAYGRHMQDAEMFTNAASMALNIWDRFDVFSTLGATSGYLKGNSASFNLVGLFGDNENQKTVKAESVPNMSFDQSVVELYTDTTFAWSVGARAALWESGSATLGASFQYAQSKPKVEELNVLSNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 65 - protein, 1068 aa, Chlamydia trachomatis
TVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGSRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRASRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRADAISMRVGYYGDFVFDRVLKTDVNKEFQMGAKPTTDTGNSAAPSTLTARENPAYGRHMQDAEMFTNAASMALNIWDRFDVFSTLGATSGYLKGNSASFNLVGLFGDNENQKTVKAESVPNMSFDQSVVELYTDTTFAWSVGARAALWESGSATLGASFQYAQSKPKVEELNVLSNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 66 - protein, 606 aa, Chlamydia trachomatis
SRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRASRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRANMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 67 - protein, 1093 aa, Chlamydia trachomatis
SRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRASRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRAGIAHTEWESVIGLEVHVELNTESKLFSPARNHFGDEPNTNISPVCTGMPGSLPVLNKDAVRKAVLFGCAVEGDVALFSRFDRKSYFYPDSPRNFQITQYEHPIVRGGCIRAVVEGEEKTFELAQTHLEDDAGMLKHFGDFAGVDYNRAGVPLIEIVSKPCMFSAEDAVAYANALVSILGYIGISDCNMEEGSIRFDVNISVRPRGSRELRNKVEIKNMNSFTFMAQALEAEKRRQIEEYLSYPNEDPKKVVPAATYRWDPEKKKTVLMRLKERAEDYMYFVEPDLPVLQITETYIDEVRQTLPELPHSKYMRYITDFDIAEDLAMILVGDRHTAHFFETATMSCKNYRALSNWITVEFAGRCKARGKTLPFTGILPEWVAQLVNFIDRGVITGKIAKEIADRMVSSFGESPEDILRRHPSLLPMTDDHALRAIVKEVVAQNTASVADYKNGKAKALGFLVGQIMKRTEGKAPPKRVNELLLAAMRDMNMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 68 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 69 - protein, 122 aa, Chlamydia trachomatis
SATAIFDTTTLNPTIAGAGDVKTGAEGQLGSATAIFDTTTLNPTIAGAGDVKASAEGQLGLVTPVVDITTLNPTIAGSGSVAGANTEGQISLAKPVVDITTLNPTIAGSGSVVAANSEGQIS

SEQ ID NO: 70 - protein, 178 aa, Chlamydia trachomatis
IRIAQPKSATAIFDTTTLNPTIAGAGDVKTGAEGQLGDTMQIVSIRIAQPKSATAIFDTTTLNPTIAGAGDVKASAEGQLGDTMQIVSIRIAQPRLVTPVVDITTLNPTIAGSGSVAGANTEGQISDTMQIVSIRIAQPKLAKPVVDITTLNPTIAGSGSVVAANSEGQISDTMQIVS

SEQ ID NO: 71 - protein, 393 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGDKPTSTTGNATAPTTLTARENPAYGRHMQDAEMFTNAACMALNIWDRFDVFCTLGASSGYLKGNSASFNLVGLFGDNENQSTVKTNSVPNMSLDQSVVELYTDTAFSWSVGARAALWECGCATLGASFQYAQSKPKVEELNVLCNAAEFTINKPKGYVGQEFPLALIAGTDAATGTKDASIDYHEWQASLALSYRLNMFTPYIGVKWSRASFDADTIRIAQPKSATAIFDTTTLNPTIAGAGDVKASAEGQLGDTMQIVSLQLNKMKSRKSCGIAVGTTIVDADKYAVTVETRLIDERAAHVNAQFRF

SEQ ID NO: 72 - protein, 395 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFEMGEALAGASGNTTSTLSKLVERTNPAYGKHMQDAEMFTNAACMTLNIWDRFDVFCTLGATSGYLKGNSASFNLVGLFGDGVNATKPAADSIPNVQLNQSVVELYTDTTFAWSVGARAALWECGCATLGASFQYAQSKPKIEELNVLCNAAEFTINKPKGYVGKEFPLDLTAGTDAATGTKDASIDYHEWQASLSLSYRLNMFTPYIGVKWSRASFDSDTIRIAQPRLVTPVVDITTLNPTIAGCGSVAGANTEGQISDTMQIVSLQLNKMKSRKSCGIAVGTTIVDADKYAVTVETRLIDERAAHVNAQFRF

SEQ ID NO: 73 - protein, 393 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFEMGEALAGASGNTTSTLSKLVERTNPAYGKHMQDAEMFTNAACMALNIWDRFDVFCTLGATSGYLRGNSASFNLVGLFGDSENATQPAATSIPNVQLNQSVVELYTDTAFAWSVGARAALWECGCATLGASFQYAQSKPKVEELNVLCNAAEFTINKPKGYVGQEFPLALTAGTDAATGTKDASIDYHEWQASLSLSYRLNMFTPYIGVKWSRASFDSNTIRIAQPKLAKPVVDITTLNPTIAGCGSVVAANSEGQISDTMQIVSLQLNKMKSRKSCGIAVGTTIVDADKYAVTVETRLIDERAAHVNAQF

SEQ ID NO: 74 - protein, 389 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGAAPTTKDIAGLENDPTTNVARPNPAYGKHMQDAEMFTNAAYMALNIWDRFDVFCTLGATTGYLKGNSASFNLVGLFGTKTQSSNFNTAKLIPNAALNQAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSDNDLADTMQIVSLQLNKMKSRKSCGIAVGTTIVDADKYAVTVETRLIDERAA

SEQ ID NO: 75 - protein, 397 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGAAPTTSDVAGLQNDPTTNVARPNPAYGKHMQDAEMFTNAAYMALNIWDRFDVFCTLGATTGYLKGNSASFNLVGLFGTKTQASSFNTANLFPNTALNQAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSENDLADTMQIVSLQLNKMKSRKSCGIAVGTTIVDADKYAVTVETRLIDERAAHVNAQFRF

SEQ ID NO: 76 - protein, 9 aa, Chlamydia trachomatis
TTLNPTIAG

SEQ ID NO: 77 - protein, 27 aa, Chlamydia trachomatis
SMRVGYYGDFVFDRVLKTDVNKEFQMG

SEQ ID NO: 78 - protein, 26 aa, Chlamydia trachomatis
NPAYGRHMQDAEMFTNAACMALNIWD

SEQ ID NO: 79 - protein, 8 aa, Chlamydia trachomatis
SATAIFDT

SEQ ID NO: 80 - protein, 8 aa, Chlamydia trachomatis
LVTPVVDI

SEQ ID NO: 81 - protein, 8 aa, Chlamydia trachomatis
LAKPVVDI

SEQ ID NO: 82 - protein, 8 aa, Chlamydia trachomatis
LAEAILDV

SEQ ID NO: 83 - protein, 13 aa, Chlamydia trachomatis
AGDVKTGAEGQLG

SEQ ID NO: 84 - protein, 13 aa, Chlamydia trachomatis
AGDVKASAEGQLG

SEQ ID NO: 85 - protein, 14 aa, Chlamydia trachomatis
CGSVAGANTEGQIS

SEQ ID NO: 86 - protein, 14 aa, Chlamydia trachomatis
CGSVVAANSEGQIS

SEQ ID NO: 87 - protein, 13 aa, Chlamydia trachomatis
KGTVVSSAENELA

SEQ ID NO: 88 - protein, 14 aa, Chlamydia trachomatis
KGTVVASGSENDLA

SEQ ID NO: 89 - protein, 20 aa, Chlamydia trachomatis
DASIDYHEWQASLALSYRLN

SEQ ID NO: 90 - protein, 20 aa, Chlamydia trachomatis
ASLALSYRLNMFTPYIGVKW

SEQ ID NO: 91 - protein, 20 aa, Chlamydia trachomatis
MFTPYIGVKWSRASFDADTI

SEQ ID NO: 92 - protein, 20 aa, Chlamydia trachomatis
SRASFDADTIRIAQPKSATA

SEQ ID NO: 93 - protein, 20 aa, Chlamydia trachomatis
RIAQPKSATAIFDTTTLNPT

SEQ ID NO: 94 - protein, 20 aa, Chlamydia trachomatis
IFDTTTLNPTIAGAGDVKAS

SEQ ID NO: 95 - protein, 20 aa, Chlamydia trachomatis
IAGAGDVKASAEGQLGDTMQ

SEQ ID NO: 96 - protein, 20 aa, Chlamydia trachomatis
AEGQLGDTMQIVSLQLNKMK

SEQ ID NO: 97 - protein, 20 aa, Chlamydia trachomatis
DASIDYHEWQASLSLSYRLN

SEQ ID NO: 98 - protein, 20 aa, Chlamydia trachomatis
ASLSLSYRLNMFTPYIGVKW

SEQ ID NO: 99 - protein, 20 aa, Chlamydia trachomatis
MFTPYIGVKWSRASFDSDTI

SEQ ID NO: 100 - protein, 20 aa, Chlamydia trachomatis
SRASFDSDTIRIAQPRLVTP

SEQ ID NO: 101 - protein, 20 aa, Chlamydia trachomatis
RIAQPRLVTPVVDITTLNPT

SEQ ID NO: 102 - protein, 20 aa, Chlamydia trachomatis
VVDITTLNPTIAGCGSVAGA

SEQ ID NO: 103 - protein, 21 aa, Chlamydia trachomatis
IAGCGSVAGANTEGQISDTMQ

SEQ ID NO: 104 - protein, 20 aa, Chlamydia trachomatis
TEGQISDTMQIVSLQLNKMK

SEQ ID NO: 105 - protein, 9 aa, Chlamydia trachomatis
SRASFDADT

SEQ ID NO: 106 - protein, 9 aa, Chlamydia trachomatis
RASFDADTI

SEQ ID NO: 107 - protein, 9 aa, Chlamydia trachomatis
ASFDADTIR

SEQ ID NO: 108 - protein, 9 aa, Chlamydia trachomatis
SFDADTIRI

SEQ ID NO: 109 - protein, 9 aa, Chlamydia trachomatis
FDADTIRIA

SEQ ID NO: 110 - protein, 9 aa, Chlamydia trachomatis
DADTIRIAQ

SEQ ID NO: 111 - protein, 9 aa, Chlamydia trachomatis
ADTIRIAQP

SEQ ID NO: 112 - protein, 9 aa, Chlamydia trachomatis
DTIRIAQPK

SEQ ID NO: 113 - protein, 9 aa, Chlamydia trachomatis
TIRIAQPKS

SEQ ID NO: 114 - protein, 9 aa, Chlamydia trachomatis
IRIAQPKSA

SEQ ID NO: 115 - protein, 9 aa, Chlamydia trachomatis
RIAQPKSAT

SEQ ID NO: 116 - protein, 9 aa, Chlamydia trachomatis
IAQPKSATA

SEQ ID NO: 117 - protein, 9 aa, Chlamydia trachomatis
AQPKSATAI

SEQ ID NO: 118 - protein, 9 aa, Chlamydia trachomatis
QPKSATAIF

SEQ ID NO: 119 - protein, 9 aa, Chlamydia trachomatis
PKSATAIFD

SEQ ID NO: 120 - protein, 9 aa, Chlamydia trachomatis
KSATAIFDT

SEQ ID NO: 121 - protein, 9 aa, Chlamydia trachomatis
SATAIFDTT

SEQ ID NO: 122 - protein, 9 aa, Chlamydia trachomatis
ATAIFDTTT

SEQ ID NO: 123 - protein, 9 aa, Chlamydia trachomatis
TAIFDTTTL

SEQ ID NO: 124 - protein, 9 aa, Chlamydia trachomatis
AIFDTTTLN

SEQ ID NO: 125 - protein, 9 aa, Chlamydia trachomatis
IFDTTTLNP

SEQ ID NO: 126 - protein, 9 aa, Chlamydia trachomatis
FDTTTLNPT

SEQ ID NO: 127 - protein, 9 aa, Chlamydia trachomatis
DTTTLNPTI

SEQ ID NO: 128 - protein, 9 aa, Chlamydia trachomatis
TTTLNPTIA

SEQ ID NO: 129 - protein, 9 aa, Chlamydia trachomatis
TLNPTIAGA

SEQ ID NO: 130 - protein, 9 aa, Chlamydia trachomatis
LNPTIAGAG

SEQ ID NO: 131 - protein, 9 aa, Chlamydia trachomatis
NPTIAGAGD

SEQ ID NO: 132 - protein, 9 aa, Chlamydia trachomatis
PTIAGAGDV

SEQ ID NO: 133 - protein, 9 aa, Chlamydia trachomatis
TIAGAGDVK

SEQ ID NO: 134 - protein, 9 aa, Chlamydia trachomatis
IAGAGDVKT

SEQ ID NO: 135 - protein, 9 aa, Chlamydia trachomatis
AGAGDVKTG

SEQ ID NO: 136 - protein, 9 aa, Chlamydia trachomatis
GAGDVKTGA

SEQ ID NO: 137 - protein, 9 aa, Chlamydia trachomatis
AGDVKTGAE

SEQ ID NO: 138 - protein, 9 aa, Chlamydia trachomatis
GDVKTGAEG

SEQ ID NO: 139 - protein, 9 aa, Chlamydia trachomatis
DVKTGAEGQ

SEQ ID NO: 140 - protein, 9 aa, Chlamydia trachomatis
VKTGAEGQL

SEQ ID NO: 141 - protein, 9 aa, Chlamydia trachomatis
KTGAEGQLG

SEQ ID NO: 142 - protein, 9 aa, Chlamydia trachomatis
TGAEGQLGD

SEQ ID NO: 143 - protein, 9 aa, Chlamydia trachomatis
GAEGQLGDT

SEQ ID NO: 144 - protein, 9 aa, Chlamydia trachomatis
AEGQLGDTM

SEQ ID NO: 145 - protein, 9 aa, Chlamydia trachomatis
EGQLGDTMQ

SEQ ID NO: 146 - protein, 9 aa, Chlamydia trachomatis
GQLGDTMQI

SEQ ID NO: 147 - protein, 9 aa, Chlamydia trachomatis
QLGDTMQIV

SEQ ID NO: 148 - protein, 9 aa, Chlamydia trachomatis
LGDTMQIVS

SEQ ID NO: 149 - protein, 9 aa, Chlamydia trachomatis
SRASFDSDT

SEQ ID NO: 150 - protein, 9 aa, Chlamydia trachomatis
RASFDSDTI

SEQ ID NO: 151 - protein, 9 aa, Chlamydia trachomatis
ASFDSDTIR

SEQ ID NO: 152 - protein, 9 aa, Chlamydia trachomatis
SFDSDTIRI

SEQ ID NO: 153 - protein, 9 aa, Chlamydia trachomatis
FDSDTIRIA

SEQ ID NO: 154 - protein, 9 aa, Chlamydia trachomatis
DSDTIRIAQ

SEQ ID NO: 155 - protein, 9 aa, Chlamydia trachomatis
SDTIRIAQP

SEQ ID NO: 156 - protein, 9 aa, Chlamydia trachomatis
DTIRIAQPR

SEQ ID NO: 157 - protein, 9 aa, Chlamydia trachomatis
TIRIAQPRL

SEQ ID NO: 158 - protein, 9 aa, Chlamydia trachomatis
IRIAQPRLV

SEQ ID NO: 159 - protein, 9 aa, Chlamydia trachomatis
RIAQPRLVT

SEQ ID NO: 160 - protein, 9 aa, Chlamydia trachomatis
IAQPRLVTP

SEQ ID NO: 161 - protein, 9 aa, Chlamydia trachomatis
AQPRLVTPV

SEQ ID NO: 162 - protein, 9 aa, Chlamydia trachomatis
QPRLVTPVV

SEQ ID NO: 163 - protein, 9 aa, Chlamydia trachomatis
PRLVTPVVD

SEQ ID NO: 164 - protein, 9 aa, Chlamydia trachomatis
RLVTPVVDI

SEQ ID NO: 165 - protein, 9 aa, Chlamydia trachomatis
LVTPVVDIT

SEQ ID NO: 166 - protein, 9 aa, Chlamydia trachomatis
VTPVVDITT

SEQ ID NO: 167 - protein, 9 aa, Chlamydia trachomatis
TPVVDITTL

SEQ ID NO: 168 - protein, 9 aa, Chlamydia trachomatis
PVVDITTLN

SEQ ID NO: 169 - protein, 9 aa, Chlamydia trachomatis
VVDITTLNP

SEQ ID NO: 170 - protein, 9 aa, Chlamydia trachomatis
VDITTLNPT

SEQ ID NO: 171 - protein, 9 aa, Chlamydia trachomatis
DITTLNPTI

SEQ ID NO: 172 - protein, 9 aa, Chlamydia trachomatis
ITTLNPTIA

SEQ ID NO: 173 - protein, 9 aa, Chlamydia trachomatis
TLNPTIAGC

SEQ ID NO: 174 - protein, 9 aa, Chlamydia trachomatis
LNPTIAGCG

SEQ ID NO: 175 - protein, 9 aa, Chlamydia trachomatis
NPTIAGCGS

SEQ ID NO: 176 - protein, 9 aa, Chlamydia trachomatis
PTIAGCGSV

SEQ ID NO: 177 - protein, 9 aa, Chlamydia trachomatis
TIAGCGSVA

SEQ ID NO: 178 - protein, 9 aa, Chlamydia trachomatis
IAGCGSVAG

SEQ ID NO: 179 - protein, 9 aa, Chlamydia trachomatis
AGCGSVAGA

SEQ ID NO: 180 - protein, 9 aa, Chlamydia trachomatis
GCGSVAGAN

SEQ ID NO: 181 - protein, 9 aa, Chlamydia trachomatis
CGSVAGANT

SEQ ID NO: 182 - protein, 9 aa, Chlamydia trachomatis
GSVAGANTE

SEQ ID NO: 183 - protein, 9 aa, Chlamydia trachomatis
SVAGANTEG

SEQ ID NO: 184 - protein, 9 aa, Chlamydia trachomatis
VAGANTEGQ

SEQ ID NO: 185 - protein, 9 aa, Chlamydia trachomatis
AGANTEGQI

SEQ ID NO: 186 - protein, 9 aa, Chlamydia trachomatis
GANTEGQIS

SEQ ID NO: 187 - protein, 9 aa, Chlamydia trachomatis
ANTEGQISD

SEQ ID NO: 188 - protein, 9 aa, Chlamydia trachomatis
NTEGQISDT

SEQ ID NO: 189 - protein, 9 aa, Chlamydia trachomatis
TEGQISDTM

SEQ ID NO: 190 - protein, 9 aa, Chlamydia trachomatis
EGQISDTMQ

SEQ ID NO: 191 - protein, 9 aa, Chlamydia trachomatis
GQISDTMQI

SEQ ID NO: 192 - protein, 9 aa, Chlamydia trachomatis
QISDTMQIV

SEQ ID NO: 193 - protein, 9 aa, Chlamydia trachomatis
ISDTMQIVS

SEQ ID NO: 194 - protein, 17 aa, Chlamydia trachomatis
FDTTTLNPTIAGAGDVK

SEQ ID NO: 195 - protein, 9 aa, Chlamydia trachomatis
NMFTPYIGV

SEQ ID NO: 196 - protein, 9 aa, Chlamydia trachomatis
MFTPYIGVK

SEQ ID NO: 197 - protein, 9 aa, Chlamydia trachomatis
FTPYIGVKW

SEQ ID NO: 198 - protein, 9 aa, Chlamydia trachomatis
TPYIGVKWS

SEQ ID NO: 199 - protein, 9 aa, Chlamydia trachomatis
PYIGVKWSR

SEQ ID NO: 200 - protein, 9 aa, Chlamydia trachomatis
YIGVKWSRA

SEQ ID NO: 201 - protein, 9 aa, Chlamydia trachomatis
IGVKWSRAS

SEQ ID NO: 202 - protein, 9 aa, Chlamydia trachomatis
GVKWSRASF

SEQ ID NO: 203 - protein, 9 aa, Chlamydia trachomatis
VKWSRASFD

SEQ ID NO: 204 - protein, 9 aa, Chlamydia trachomatis
KWSRASFDA

SEQ ID NO: 205 - protein, 9 aa, Chlamydia trachomatis
WSRASFDAD

SEQ ID NO: 206 - protein, 9 aa, Chlamydia trachomatis
SRASFDADT

SEQ ID NO: 207 - protein, 9 aa, Chlamydia trachomatis
RASFDADTI

SEQ ID NO: 208 - protein, 9 aa, Chlamydia trachomatis
ASFDADTIR

SEQ ID NO: 209 - protein, 9 aa, Chlamydia trachomatis
SFDADTIRI

SEQ ID NO: 210 - protein, 9 aa, Chlamydia trachomatis
FDADTIRIA

SEQ ID NO: 211 - protein, 9 aa, Chlamydia trachomatis
DADTIRIAQ

SEQ ID NO: 212 - protein, 9 aa, Chlamydia trachomatis
ADTIRIAQP

SEQ ID NO: 213 - protein, 9 aa, Chlamydia trachomatis
DTIRIAQPK

SEQ ID NO: 214 - protein, 9 aa, Chlamydia trachomatis
TIRIAQPKS

SEQ ID NO: 215 - protein, 9 aa, Chlamydia trachomatis
IRIAQPKSA

SEQ ID NO: 216 - protein, 9 aa, Chlamydia trachomatis
RIAQPKSAT

SEQ ID NO: 217 - protein, 9 aa, Chlamydia trachomatis
IAQPKSATA

SEQ ID NO: 218 - protein, 9 aa, Chlamydia trachomatis
AQPKSATAI

SEQ ID NO: 219 - protein, 9 aa, Chlamydia trachomatis
QPKSATAIF

SEQ ID NO: 220 - protein, 9 aa, Chlamydia trachomatis
PKSATAIFD

SEQ ID NO: 221 - protein, 9 aa, Chlamydia trachomatis
KSATAIFDT

SEQ ID NO: 222 - protein, 9 aa, Chlamydia trachomatis
SATAIFDTT

SEQ ID NO: 223 - protein, 9 aa, Chlamydia trachomatis
ATAIFDTTT

SEQ ID NO: 224 - protein, 9 aa, Chlamydia trachomatis
TAIFDTTTL

SEQ ID NO: 225 - protein, 9 aa, Chlamydia trachomatis
AIFDTTTLN

SEQ ID NO: 226 - protein, 9 aa, Chlamydia trachomatis
IFDTTTLNP

SEQ ID NO: 227 - protein, 9 aa, Chlamydia trachomatis
FDTTTLNPT

SEQ ID NO: 228 - protein, 9 aa, Chlamydia trachomatis
DTTTLNPTI

SEQ ID NO: 229 - protein, 9 aa, Chlamydia trachomatis
TTTLNPTIA

SEQ ID NO: 230 - protein, 9 aa, Chlamydia trachomatis
TTLNPTIAG

SEQ ID NO: 231 - protein, 9 aa, Chlamydia trachomatis
TLNPTIAGA

SEQ ID NO: 232 - protein, 9 aa, Chlamydia trachomatis
LNPTIAGAG

SEQ ID NO: 233 - protein, 9 aa, Chlamydia trachomatis
NPTIAGAGD

SEQ ID NO: 234 - protein, 9 aa, Chlamydia trachomatis
PTIAGAGDV

SEQ ID NO: 235 - protein, 9 aa, Chlamydia trachomatis
TIAGAGDVK

SEQ ID NO: 236 - protein, 9 aa, Chlamydia trachomatis
IAGAGDVKT

SEQ ID NO: 237 - protein, 9 aa, Chlamydia trachomatis
AGAGDVKTG

SEQ ID NO: 238 - protein, 9 aa, Chlamydia trachomatis
GAGDVKTGA

SEQ ID NO: 239 - protein, 9 aa, Chlamydia trachomatis
AGDVKTGAE

SEQ ID NO: 240 - protein, 9 aa, Chlamydia trachomatis
GDVKTGAEG

SEQ ID NO: 241 - protein, 9 aa, Chlamydia trachomatis
DVKTGAEGQ

SEQ ID NO: 242 - protein, 9 aa, Chlamydia trachomatis
VKTGAEGQL

SEQ ID NO: 243 - protein, 9 aa, Chlamydia trachomatis
KTGAEGQLG

SEQ ID NO: 244 - protein, 9 aa, Chlamydia trachomatis
TGAEGQLGD

SEQ ID NO: 245 - protein, 9 aa, Chlamydia trachomatis
GAEGQLGDT

SEQ ID NO: 246 - protein, 9 aa, Chlamydia trachomatis
AEGQLGDTM

SEQ ID NO: 247 - protein, 9 aa, Chlamydia trachomatis
EGQLGDTMQ

SEQ ID NO: 248 - protein, 9 aa, Chlamydia trachomatis
GQLGDLMQI

SEQ ID NO: 249 - protein, 9 aa, Chlamydia trachomatis
QLGDTMQIV

SEQ ID NO: 250 - protein, 9 aa, Chlamydia trachomatis
LGDTMQIVS

SEQ ID NO: 251 - protein, 9 aa, Chlamydia trachomatis
GDTMQIVSL

SEQ ID NO: 252 - protein, 9 aa, Chlamydia trachomatis
DTMQIVSLQ

SEQ ID NO: 253 - protein, 9 aa, Chlamydia trachomatis
TMQIVSLQL

SEQ ID NO: 254 - protein, 9 aa, Chlamydia trachomatis
MQIVSLQLN

SEQ ID NO: 255 - protein, 80 aa, Chlamydia trachomatis
EWQASLALSYRLNMFTPYIGVKWSRASFDADTIRIAQPKSATAIFDTTTLNPTIAGAGDVKTGAEGQLGDTMQIVSLQLN